SEQUENCE LISTING (11 sequences)

SEQ ID NO: 1 - protein, 508 aa, synthetic construct
MEPAGPAPGRLGPLLLCLLLSASCFCTGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAADNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 2 - protein, 293 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 3 - protein, 305 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRNR

SEQ ID NO: 4 - protein, 312 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRKAVEEPLNE

SEQ ID NO: 5 - protein, 323 aa, synthetic construct
MWPLAAALLLGSCCCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQRTIQPPRKAVEEPLNAFKESKGMMNDE

SEQ ID NO: 6 - DNA, 22 bp, synthetic construct
  source 1..22: mol_type = other DNA
cttctcttctgagggggtgcaa

SEQ ID NO: 7 - DNA, 20 bp, synthetic construct
  source 1..20: mol_type = other DNA
gccatgtgtgtgtttatgtc

SEQ ID NO: 8 - DNA, 20 bp, synthetic construct
  source 1..20: mol_type = other DNA
acagttccaacccatgggtg

SEQ ID NO: 9 - DNA, 22 bp, synthetic construct
  source 1..22: mol_type = other DNA
cctctggttcctcctccacaag

SEQ ID NO: 10 - DNA, 24 bp, synthetic construct
  source 1..24: mol_type = other DNA
tgaagacatgtggctgccatccag

SEQ ID NO: 11 - DNA, 26 bp, synthetic construct
  source 1..26: mol_type = other DNA
cgtgtctgcattgctatgagatgggt

(19) United States
(12) Patent Application Publication
(10) Pub. No.: US 2025/0255282 A1
(43) Pub. Date: Aug. 14, 2025

(54) VECTORS, GENETICALLY MODIFIED CELLS, AND GENETICALLY MODIFIED NON-HUMAN ANIMALS COMPRISING THE SAME

(71) Applicant: Regeneron Pharmaceuticals, Inc., Tarrytown, NY (US)

(72) Inventors: Davor Frleta, Forest Hills, NY (US); Naxin Tu, Pleasantville, NY (US)

(21) Appl. No.: 19/048,642

(22) Filed: Feb. 7, 2025

Related U.S. Application Data
(60) Provisional application No. 63/551,191, filed on Feb. 8, 2024.

(51) Int. Cl. A01K 67/0278; A01K 67/0271; A01K 67/0276; C07K 14/47; C07K 14/52; C07K 14/535; C07K 14/54; C07K 14/55; C07K 14/705

(52) U.S. Cl. CPC A01K 67/0278; A01K 67/0271; A01K 67/0276; C07K 14/47; C07K 14/52; C07K 14/535; C07K 14/5403; C07K 14/55; C07K 14/70503; A01K 2207/12; A01K 2207/15; A01K 2217/072; A01K 2217/075; A01K 2227/105; A01K 2267/03

(57) ABSTRACT

Provided herein are genetically modified cells and genetically modified non-human animals (e.g., rodents such as rats and mice) comprising: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, and optionally expressing one or more human or humanized polypeptides. Methods and compositions of making and using such genetically modified cells and non-human animals are also provided.

### RELATED APPLICATIONS

This application claims priority to U.S. Provisional Application No. 63/551,191, filed Feb. 8, 2024, which is hereby incorporated herein by reference in its entirety.

### SEQUENCE LISTING

The instant application contains a Sequence Listing which has been submitted electronically in XML format and is hereby incorporated by reference in its entirety. Said XML copy, created on Jan. 27, 2025, is named RPD-00501_SL.xml and is 12,204 bytes in size.

### BACKGROUND

Genetically modified cells, genetically modified mice comprising the same, as well as modified and engrafted mice, and their use in modeling human diseases, e.g., for the purpose of drug testing, are known in the art. Use of genetically modified mice to model a human immune system (HIS) has been reported (Manz (2007) Immunity, 26:537-541). For example, HIS mice generated by transplanting a severely immunodeficient mouse strain (such as Rag2 KO Il2rg KO mice) with human hematopoietic stem and progenitor cells have been reported.

Neutrophils are the most abundant leukocytes in the human blood and are critical for control of bacterial and fungal infections. Neutrophils mediate protection against pathogens through several mechanisms including phagocytosis, release of lytic enzymes and defensins, production of reactive oxygen intermediates, and entrapment via extracellular nets comprised of self-DNA. Immuno-deficient mice engrafted with human hematopoietic stem cells (HSCs) allow for the development of a working human immune system but many such HIS mouse models have inadequate development of human neutrophils. Whereas normal human blood has 1500-8000 neutrophils per mL, most HIS mouse models have <10 neutrophils per mL of blood. Recently, the advanced MISTRG mouse model has been reported to have increased human neutrophil development in the bone marrow (BM), but further improvement in neutrophil levels in the periphery (spleen/blood) are desirable. MISTRG has previously been described to increase human myeloid cell development as well as be a superior model for patient derived xenograft (PDX) tumor engraftment and HIS engraftment. This is primarily due to humanization of various cytokines such as M-CSF, GM-CSF, IL-3 which potentiate myeloid/granulocyte development, thrombopoietin (TPO) which increases human stem cell survival, and SIRPa which increases overall human leukocyte engraftment by inhibiting murine macrophage phagocytosis of donor human stem cells. Thus, further modifications are needed to improve human neutrophil development in the periphery.

There is a need for genetically modified mice that can support human neutrophil cell development, and for mice suitable for engraftment that can model or approximate certain aspects of a human neutrophil cells.

### SUMMARY

The present disclosure is based, in part, on the generation of a HIS model that couples humanization of GM-CSF/IL-3, cytokines important for granulocyte development, with deletion of murine Csf2rb and Csf2rb2 genes that encode the receptors for GM-CSF and/or IL-3. The present disclosure shows that engraftment of this model with human HSCs allowed for increased levels of human neutrophils in spleen/blood as well as in the BM.

In some aspects, provided herein is a genetically modified non-human animal, comprising: (i) a homozygous null mutation in Rag2 gene; (ii) a homozygous null mutation in IL2rg gene; (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter.

In some embodiments, the genetically modified non-human animal provided herein comprises a homozygous null mutation in Rag1 gene.

In some embodiments, the genetically modified non-human animal provided herein comprises a homozygous null mutation in the non-human animal Csf2rb gene. In some embodiments, the null mutation in the non-human animal Csf2rb gene comprises an insertion, a deletion, and/or a substitution in the endogenous Csf2rb gene. In some embodiments, the null mutation in the non-human animal Csf2rb gene is a deletion of the full Csf2rb endogenous coding sequence. In some embodiments, the genetically modified non-human animal is a mouse, and the mouse comprises a homozygous null mutation in colony stimulating factor 2 receptor subunit beta 2 (Csf2rb2) gene. In some embodiments, the null mutation in Csf2rb gene comprises an insertion, a deletion, and/or a substitution in the endogenous Csf2rb2 gene. In some embodiments, the null mutation in Csf2rb2 gene is a deletion of the full Csf2rb2 endogenous coding sequence. In some embodiments, the mouse comprises a homozygous null mutation in Csf2rb gene and a homozygous null mutation in Csf2rb2 gene. In some embodiments, the genetically modified non-human animal is a mouse, and the mouse comprises a homozygous deletion of nucleic acid sequence between coordinates chr15: 78,282,507-78,351,090 (GRCm38.p6 assembly). In some embodiments, the null mutation in Csf2rb gene is a deletion of the full Csf2rb endogenous coding sequence, and the null mutation in Csf2rb2 gene is a deletion of the full Csf2rb2 endogenous coding sequence.

In some embodiments, the genetically modified non-human animal expresses a human GM-CSF protein. In some embodiments, the genetically modified non-human animal expresses a human IL-3 protein.

In some embodiments, the genetically modified non-human animal expresses a human or humanized SIRPA polypeptide encoded by a nucleic acid operably linked to a Sirpa promoter. In some embodiments, the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter. In some embodiments, the Sirpa gene comprises exons 2-4 of a human SIRPA gene. In some embodiments, the genetically modified non-human animal expresses a Sirpa polypeptide comprising an extracellular portion of a human SIRPA polypeptide and an intracellular portion of a non-human animal Sirpa polypeptide. In some embodiments, the non-human animal Sirpa polypeptide is an endogenous non-human animal Sirpa polypeptide, and/or the non-human animal Sirpa gene is an endogenous non-human animal gene. In some embodiments, the genetically modified non-human animal expresses a human SIRPA polypeptide encoded by a nucleic acid operably linked to a Sirpa promoter.

In some embodiments, the genetically modified non-human animal further expresses one or more human or humanized proteins selected from the group consisting of: a human TPO protein encoded by a nucleic acid operably linked to a TPO promoter; a human M-CSF protein encoded by a nucleic acid operably linked to an M-CSF promoter; and a human or humanized CD47 protein encoded by a nucleic acid operably linked to a CD47 promoter.

In some embodiments, at least one promoter operably linked to a nucleic acid that encodes a human or humanized protein is an endogenous non-human animal promoter. In some embodiments, all promoters operably linked to the nucleic acids that encode the human or humanized proteins are endogenous non-human animal promoters. In some embodiments, the endogenous non-human animal promoter is at the corresponding non-human animal gene locus. In some embodiments, the genetically modified non-human animal provided herein comprises a null mutation in at least one corresponding non-human animal gene at the corresponding non-human animal gene locus. In some embodiments, the genetically modified non-human animal is heterozygous for at least one allele comprising the nucleic acid sequence that encodes the human or humanized protein. In some embodiments, the genetically modified non-human animal is homozygous for at least one allele comprising the nucleic acid sequence that encodes the human or humanized protein.

In some embodiments, the genetically modified non-human animal expresses a human M-CSF protein encoded by a nucleic acid operably linked to an M-CSF promoter. In some embodiments, the genetically modified non-human animal expresses a human or humanized CD47 protein encoded by a nucleic acid operably linked to a CD47 promoter. In some embodiments, the genetically modified non-human animal expresses a humanized CD47 protein, and the humanized CD47 protein comprises an extracellular portion of a human CD47 protein and an intracellular portion of an endogenous non-human animal CD47 protein. In some embodiments, the genetically modified non-human animal expresses a human TPO protein encoded by a nucleic acid operably linked to a TPO promoter. In some embodiments, the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; (iii) a human or humanized CD47 protein encoded by a nucleic acid operably linked to a CD47 promoter; and (iv) a human TPO protein encoded by a nucleic acid operably linked to a TPO promoter.

In some embodiments, the genetically modified non-human animal further comprises an engraftment of human hematopoietic cells. In some embodiments, the human hematopoietic cells comprise one or more cells selected from the group consisting of a human CD34-positive cell, a human hematopoietic stem cell, a human hematopoietic progenitor cell, a human neutrophil precursor cell, and a human neutrophil. In some embodiments, the genetically modified non-human animal comprises human neutrophils.

In some embodiments, anti-neutrophil cytoplasmic autoimmune (ANCA) vasculitis is established in the genetically modified non-human animal. In some embodiments, Granulomatosis with polyangiitis (GPA) is established in the genetically modified non-human animal.

In some embodiments, a tumor is established in the genetically modified non-human animal.

In some embodiments, the genetically modified non-human animal is infected by a bacterial or fungal pathogen.

In some embodiments, human neutrophil NETosis is established in the genetically modified non-human animal. In some embodiments, the human neutrophil NETosis provides an autoantigen in the genetically modified non-human animal. In some embodiments, the genetically modified non-human animal has an autoimmune disease that involves the human neutrophil NETosis.

In some embodiments, the genetically modified non-human animal is a mammal. In some embodiments, the mammal is a rodent, such as a rat or a mouse. In some embodiments, the rodent is a mouse.

In some aspects, provided herein is a method for identifying an agent that treats anti-neutrophil cytoplasmic autoimmune (ANCA) vasculitis, the method comprising: (a) administering the agent to a genetically modified non-human animal provided herein; and (b) determining whether the agent treats ANCA vasculitis in the non-human animal.

In some aspects, provided herein is a method for identifying an agent that treats Granulomatosis with polyangiitis (GPA), the method comprising: (a) administering the agent to a genetically modified non-human animal provided herein; and (b) determining whether the agent treats GPA in the non-human animal.

In some aspects, provided herein is a method for identifying an agent that mitigates tumor progression, the method comprising: (a) administering the agent to a genetically modified non-human animal provided herein; and (b) determining whether the agent mitigates tumor progression in the non-human animal.

In some aspects, provided herein is a method for identifying an agent that inhibits an infection by a bacterial or fungal pathogen, the method comprising: (a) administering the agent to a genetically modified non-human animal provided herein, and (b) determining whether the agent reduces the amount of the pathogen and/or inhibits the activity of the pathogen in the pathogen-infected non-human animal.

In some aspects, provided herein is a method for identifying an agent that mitigates NETosis, the method comprising: (a) administering the agent to a genetically modified non-human animal provided herein; and (b) determining whether the agent mitigates NETosis in the non-human animal.

In some aspects, provided herein is a method for identifying an agent that treats an autoimmune disease that involves NETosis, the method comprising: (a) administering the agent to a genetically modified non-human animal provided herein; and (b) determining whether the agent treats the autoimmune disease in the non-human animal.

In some embodiments, the agent targets human neutrophils.

In some aspects, provided herein is a method for assessing therapeutic efficacy of a drug candidate to modulate a function of a human neutrophil, the method comprising: (a) administering the drug candidate to a genetically modified non-human animal provided herein; and (b) determining whether the drug candidate modulates the function of the human neutrophil in the genetically modified non-human animal.

In some embodiments, the drug candidate is an agent targeting FcαR. In some embodiments, the function of the human neutrophil cell is selected from a group consisting of phagocytosis, cytokine production, and activation.

In some aspects, provided herein is a genetically modified non-human animal cell, comprising: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter.

In some embodiments, the genetically modified non-human animal cell provided herein comprises a homozygous null mutation in Rag1 gene.

In some embodiments, the genetically modified non-human animal cell provided herein comprises a homozygous null mutation in the non-human animal Csf2rb gene. In some embodiments, the null mutation in the non-human animal Csf2rb gene comprises an insertion, a deletion, and/or a substitution in the endogenous Csf2rb gene. In some embodiments, the null mutation in the non-human animal Csf2rb gene is a deletion of the full Csf2rb endogenous coding sequence. In some embodiments, the genetically modified non-human animal cell is a mouse cell, and the mouse cell comprises a homozygous null mutation in colony stimulating factor 2 receptor subunit beta 2 (Csf2rb2) gene. In some embodiments, the null mutation in Csf2rb gene comprises an insertion, a deletion, and/or a substitution in the endogenous Csf2rb2 gene. In some embodiments, the null mutation in Csf2rb2 gene is a deletion of the full Csf2rb2 endogenous coding sequence. In some embodiments, the mouse cell comprises a homozygous null mutation in Csf2rb gene and a homozygous null mutation in Csf2rb2 gene. In some embodiments, the genetically modified non-human animal cell is a mouse cell, and the mouse cell comprises a homozygous deletion of nucleic acid sequence between coordinates chr15: 78,282,507-78,351,090 (GRCm38.p6 assembly). In some embodiments, the null mutation in Csf2rb gene is a deletion of the full Csf2rb endogenous coding sequence, and the null mutation in Csf2rb2 gene is a deletion of the full Csf2rb2 endogenous coding sequence.

In some embodiments, the genetically modified non-human animal cell expresses a human GM-CSF protein. In some embodiments, the genetically modified non-human animal cell expresses a human IL-3 protein.

In some embodiments, the genetically modified non-human animal cell further comprises a nucleic acid that encodes a human or humanized SIRPA polypeptide, and wherein the nucleic acid is operably linked to a Sirpa promoter. In some embodiments, the genetically modified non-human animal cell expresses a human or humanized SIRPA polypeptide. In some embodiments, the genetically modified non-human animal cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter. In some embodiments, the Sirpa gene comprises exons 2-4 of a human SIRPA gene. In some embodiments, the genetically modified non-human animal cell expresses a Sirpa polypeptide comprising an extracellular portion of a human SIRPA polypeptide and an intracellular portion of a non-human animal Sirpa polypeptide. In some embodiments, the non-human animal Sirpa polypeptide is an endogenous non-human animal Sirpa polypeptide, and/or the non-human animal Sirpa gene is an endogenous non-human animal gene. In some embodiments, the genetically modified non-human animal cell expresses a human SIRPA polypeptide.

In some embodiments, the genetically modified non-human animal cell further comprises one or more nucleic acids selected from the group consisting of: (1) a nucleic acid that encodes a human TPO protein and is operably linked to a TPO promoter; (2) a nucleic acid that encodes a human M-CSF protein and is operably linked to an M-CSF promoter; and (3) a nucleic acid that encodes a human or humanized CD47 protein and is operably linked to a CD47 promoter. In some embodiments, the genetically modified non-human animal cell expresses one or more human or humanized proteins selected from the group consisting of: a human TPO protein; a human M-CSF protein; and a human or humanized CD47 protein. In some embodiments, at least one promoter operably linked to a nucleic acid that encodes a human or humanized protein is an endogenous non-human animal promoter. In some embodiments, all promoters operably linked to the nucleic acids that encode the human or humanized proteins are endogenous non-human animal promoters. In some embodiments, the endogenous non-human animal promoter is at the corresponding non-human animal gene locus. In some embodiments, the genetically modified non-human animal cell provided herein comprises a null mutation in at least one corresponding non-human animal gene at the corresponding non-human animal gene locus. In some embodiments, the genetically modified non-human animal cell is heterozygous for at least one allele comprising the nucleic acid sequence that encodes the human or humanized protein. In some embodiments, the genetically modified non-human animal cell is homozygous for at least one allele comprising the nucleic acid sequence that encodes the human or humanized protein.

In some embodiments, the genetically modified non-human animal cell expresses a human M-CSF protein. In some embodiments, the genetically modified non-human animal cell expresses a human or humanized CD47 protein. In some embodiments, the genetically modified non-human animal cell expresses a humanized CD47 protein, and the humanized CD47 protein comprises an extracellular portion of a human CD47 protein and an intracellular portion of an endogenous non-human animal CD47 protein. In some embodiments, the genetically modified non-human animal cell expresses a human TPO protein. In some embodiments, the genetically modified non-human animal cell expresses: (i) a human or humanized SIRPA protein; (ii) a human M-CSF protein; and (iii) a human or humanized CD47 protein; and (iv) a human TPO protein.

In some embodiments, the genetically modified non-human animal cell is a mammalian cell. In some embodiments, the mammalian cell is a rodent cell, such as a rat cell or a mouse cell. In some embodiments, the rodent cell is a mouse cell. In some embodiments, the genetically modified non-human animal cell is a non-human animal embryonic stem (ES) cell.

In some aspects, provided herein is a method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, the non-human animal embryonic stem cell is a mouse embryonic stem cell, and the at least one non-human animal Csf2rb gene comprises mouse Csf2rb gene and/or mouse Csf2rb2 gene.

In some aspects, provided herein is a non-human animal embryo comprising the non-human animal embryonic stem cell provided herein, or the non-human animal embryonic stem cell made according to the method provided herein.

In some aspects, provided herein is a method of making a non-human animal comprising in its genome: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising steps of: (a) obtaining a non-human animal embryonic stem cell provided herein, or the non-human animal embryonic stem cell made according to the method provided herein; and (b) creating a non-human animal using the non-human animal embryonic cell of (a).

In some aspects, provided herein is a method of making a non-human animal comprising in its genome: ((i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising modifying the genome of the non-human animal so that it comprises: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, the non-human animal is a mouse, and the at least one non-human animal Csf2rb gene comprises mouse Csf2rb gene and/or mouse Csf2rb2 gene.

### BRIEF DESCRIPTION OF THE DRAWINGS

FIG. 1 shows schematic summaries, not to scale, of modified Csf2rb & Csf2rb2 loci according to certain embodiments provided herein.

FIGS. 2A-2D show increased human neutrophils in spleen, blood, and bone marrow (BM) of human HSC-engrafted IStRG/mCSF2Rb KO mice.

FIGS. 3A-3C show that human neutrophils from human HSC-engrafted IStRG/mCSF2Rb KO mice express CD89 (FcαR), MPO, and CD177.

FIGS. 4A-4E show gating strategy for neutrophil progenitor populations in the bone marrow of human HSC-engrafted IStRG/mCSF2Rb KO mice, and 4F shows comparable human neutrophil development in human HSC-engrafted IStRG/mCSF2Rb KO BM and in normal human BM.

### DETAILED DESCRIPTION

### General

The present disclosure relates to a genetically modified non-human animal (e.g., mouse or rat) comprising: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, provided herein is a genetically modified non-human animal (e.g., mouse) comprising: (i) a homozygous null mutation in Rag2 gene (e.g., a Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene (e.g., mouse Csf2rb gene and/or mouse Csf2rb2 gene); (iv) a nucleic acid sequence that encodes a human GM-CSF and is operably linked to a GM-CSF promoter; and (v) a nucleic acid sequence that encodes a human IL-3 and is operably linked to a IL-3 promoter.

In some embodiments, the at least one non-human animal colony stimulating factor 2 receptor subunit beta (Csf2rb) gene comprises the non-human animal Csf2rb gene and/or the non-human animal colony stimulating factor 2 receptor subunit beta 2 (Csf2rb2) gene. In some embodiments, the genetically modified non-human animal is a mouse, and the mouse comprises a homozygous null mutation in the mouse Csf2rb gene and/or the mouse Csf2rb2 gene. In some embodiments, the genetically modified non-human animal is a mouse, and the mouse comprises a homozygous null mutation in both the mouse Csf2rb gene and the mouse Csf2rb2 gene.

In some embodiments, the genetically modified non-human animal expresses a human or humanized SIRPA protein encoded by a nucleic acid operably linked to a Sirpa promoter. In some embodiments, the genetically modified non-human animal further expresses one or more human or humanized proteins selected from the group consisting of: a human TPO protein encoded by a nucleic acid operably linked to a TPO promoter; a human M-CSF protein encoded by a nucleic acid operably linked to an M-CSF promoter; and a human or humanized CD47 protein encoded by a nucleic acid operably linked to a CD47 promoter. In certain embodiments, at least one promoter operably linked to a nucleic acid that encodes a human or humanized protein is an endogenous non-human animal promoter. In other embodiments, the genetically modified animal expresses a human or humanized nucleic acid from the native human promoter and native regulatory elements. The skilled artisan will understand that the genetically modified animal includes genetically modified animals that express at least one human or humanized nucleic acid from any promoter. Examples of promoters useful in the invention include, but are not limited to, DNA pol II promoter, PGK promoter, ubiquitin promoter, albumin promoter, globin promoter, ovalbumin promoter, SV40 early promoter, the Rous sarcoma virus (RSV) promoter, retroviral LTR and lentiviral LTR. Promoter and enhancer expression systems useful in present disclosure also include inducible and/or tissue-specific expression systems.

Rodents, such as mice and rats with components of the human immune system (e.g., HIS mice and rats) hold great promise for studying the human immune system in vivo and for testing human vaccines and testing and developing drugs to treat human diseases and disorders. HIS mice are generated by transplanting a severely immunodeficient mouse strain (such as recombination-activating gene 2 (Rag2) knockout (KO) and interleukin 2 receptor gamma (IL2rg) KO mice) with human hematopoietic stem and progenitor cells (such as CD34+ HSCs). Compared to nonhuman primates, HIS mice have the advantages of a small animal model, i.e., they allow more versatile experimentation, are more accessible to the research community, and are ethically more acceptable than conducting experiments with human subjects. Most importantly, experimental findings derived from HIS mice can be more relevant and applicable to humans. Similar rat models that offer such advantages have also been described and are contemplated herein.

Although HIS mice develop human immune cells such as B and T cells, there is a need for HIS mouse models that support development of human neutrophils. Although MIStRG47 model (which includes humanized M-CSF, humanized CD47, and humanized thrombopoietin along with hSIRP and hGM-CSF/IL-3) has been published to elicit improved human granulocyte development in the BM, additional increased levels of human neutrophils in the periphery (spleen/blood) are desired. Since GM-CSF and IL-3 are critical for granulocyte development, GM-CSF/IL-3 were also humanized in the advanced MIStRG47 model and this humanization marginally increased human neutrophil levels. In contrast, it was demonstrated herein that engrafted HIS mice with humanized GM-CSF and IL-3, humanized TPO, humanized SIRPa, Rag2−/−, IL-2Rg−/− and a deletion of the murine Csf2rb & Csf2rb2 genes, mouse receptor for GM-CSF/IL-3 (IStRG/mCSF2Rb KO), exhibited increased human neutrophil levels.

The present disclosure provides a new genetically modified non-human animal (e.g., mouse) model that specifically deletes the non-human animal (e.g., mouse) receptor for GM-CSF and IL-3 (e.g., Csf2rb and/or Csf2rb2). Without wishing to be bound by theory, deleting the non-human animal (e.g., mouse) receptor for GM-CSF and IL-3 (e.g., Csf2rb and/or Csf2rb2) ablates competition for the human GM-CSF and IL-3 by non-human animal (e.g., mouse) granulocytic precursors and thus potentiates human granulocyte development in the HIS models with hGM-CSF/IL-3. The present disclosure demonstrates that this non-human animal (e.g., mouse) cytokine receptor deletion allows for increased human neutrophils in the spleen and blood at appreciably higher levels than those observed in MIStRG47. This new model provides a useful tool to study human neutrophil biology as well as a useful model for developing treatments for human neutrophil diseases.

The genetically modified non-human animals provided herein find many uses in the art, including, for example, identifying or assessing therapeutic efficacy of any therapeutic that targets neutrophils as effectors (e.g., IgA antibody therapy, FcαR targeting, etc.), as well as disease modeling in which human neutrophils are an important component (e.g., modeling anti-neutrophil cytoplasmic autoimmune (ANCA) vasculitis or neutrophil-mediated tumor progression, etc.). Additionally, the genetically modified non-human animals provided herein may find use in modeling neutrophil NETosis (which is the release of neutrophil chromatin material to ensnare microbes, and is believed to be the source of autoantigen in several autoimmune diseases that involve anti-nuclear antibodies, e.g., lupus), as well as testing therapeutic candidates in diseases that involve neutrophil NETosis. These and other objects, advantages, and features of the invention will become apparent to those persons skilled in the art upon reading the details of the compositions and methods as more fully described below.

### Definitions

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Examples of amino acids include naturally-occurring amino acids; analogs, derivatives and congeners thereof; amino acid analogs having variant side chains; and all stereoisomers of any of the foregoing.

A “coding region” of a gene includes the nucleotide residues of the coding strand of the gene and the nucleotides of the non-coding strand of the gene which are homologous with or complementary to, respectively, the coding region of an mRNA molecule which is produced by transcription of the gene. A “coding region” of a mRNA molecule also includes the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

As used herein, the phrase “endogenous gene” or “endogenous gene segment” refers to a gene or gene segment found in a parent or reference organism prior to introduction of a disruption, deletion, replacement, alteration, or modification as described herein. In some embodiments, a reference organism is a wild-type organism. In some embodiments, a reference organism is an engineered organism. In some embodiments, a reference organism is a laboratory-bred organism (whether wild-type or engineered).

As used herein, the term “chimeric” refers to nucleic acids or proteins whose structures (i.e., nucleotide or amino acid sequences) include portions that are from different species. In some embodiments, the “chimeric” nucleic acids or proteins described herein include nucleotide or amino acid sequences that are from both a non-human source (e.g., a rodent, e.g., a mouse) and a human. In such embodiments, the “chimeric” nucleic acids or proteins can also be referred to as “humanized” nucleic acids or protein.

As used herein, the term “full coding sequence” refers to a coding nucleic acid sequence ranging from the start codon to the stop codon. The term “full-length polypeptide” refers to a polypeptide that comprises an amino acid sequence that is encoded by such a full coding sequence.

The term “humanized”, is used herein in accordance with its art-understood meaning to refer to nucleic acids or proteins whose structures (i.e., nucleotide or amino acid sequences) include portions that are from a non-human source, which are engineered to have a structure and function more similar to true human nucleic acids or proteins than the original source nucleic acids or proteins. For example, humanizing can involve selecting amino acid substitutions to make a non-human sequence more similar to a human sequence. Humanizing can also involve grafting at least a portion of a non-human protein into a human protein. To give but one example, in the case of a membrane receptor, a “humanized” gene may encode a polypeptide having an extracellular portion having an amino acid sequence as that of a human extracellular portion and the remaining sequence as that of a non-human (e.g., mouse) polypeptide. In some embodiments, a humanized gene comprises at least a portion of a DNA sequence of a human gene. In some embodiments, a humanized protein comprises a sequence having a portion that appears in a human protein. The term “human” is art recognized, and refers to nucleic acids or proteins whose structures (i.e., nucleotide or amino acid sequences) are entirely from a human source.

As used herein, the term “locus” refers to a location on a chromosome that contains a set of related genetic elements (e.g., genes, gene segments, regulatory elements). A locus can be endogenous or non-endogenous. The term “endogenous locus” refers to a location on a chromosome at which a particular genetic element is naturally found. In some embodiments, an endogenous locus has a sequence found in nature. In some embodiments, an endogenous locus is a wild-type locus. In some embodiments, an endogenous locus is an engineered locus.

The phrase “non-human animal” as used herein refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human mammal is a primate, a goat, a sheep, a pig, a dog, a cow, or a rodent. In some embodiments, a non-human animal is a rodent such as a rat or a mouse.

The phrase “operably linked”, as used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to affect the expression and processing of coding sequences to which they are ligated. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “polypeptide”, as used herein, refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors. The phrase “endogenous promoter” refers to a promoter that is naturally associated, e.g., in a wild-type organism, with an endogenous gene.

The term “recombinant”, as used herein, is intended to refer to polypeptides (e.g., signal-regulatory proteins as described herein) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597, Little M. et al (2000) Immunology Today 21:364-370) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant polypeptide is comprised of sequences found in the genome of a source organism of interest (e.g., human, mouse, etc.). In some embodiments, a recombinant polypeptide has an amino acid sequence that resulted from mutagenesis (e.g., in vitro or in vivo, for example in a non-human animal), so that the amino acid sequences of the recombinant polypeptides are sequences that, while originating from and related to polypeptides sequences, may not naturally exist within the genome of a non-human animal in vivo.

The term “replacement” is used herein to refer to a process through which a “replaced” nucleic acid sequence (e.g., a gene) found in a host locus (e.g., in a genome) is removed from that locus and a different, “replacement” nucleic acid is located in its place. In some embodiments, the replaced nucleic acid sequence and the replacement nucleic acid sequences are comparable to one another in that, for example, they are homologous to one another and/or contain corresponding elements (e.g., protein-coding elements, regulatory elements, etc.). In some embodiments, a replaced nucleic acid sequence includes one or more of a promoter, an enhancer, a splice donor site, a splice receiver site, an intron, an exon, an untranslated region (UTR); in some embodiments, a replacement nucleic acid sequence includes one or more coding sequences. In some embodiments, a replacement nucleic acid sequence is a homolog of the replaced nucleic acid sequence. In some embodiments, a replacement nucleic acid sequence is an ortholog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse sequence). The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, the replacement is a substitution of an endogenous sequence with a heterologous sequence that results in the production of a gene product from the nucleic acid sequence so placed (comprising the heterologous sequence), but not expression of the endogenous sequence; the replacement is of an endogenous genomic sequence with a nucleic acid sequence that encodes a protein that has a similar function as a protein encoded by the endogenous sequence. In various embodiments, an endogenous gene or fragment thereof is replaced with a corresponding human gene or fragment thereof. A corresponding human gene or fragment thereof is a human gene or fragment that is an ortholog of, or is substantially similar or the same in structure and/or function, as the endogenous gene or fragment thereof that is replaced.

“Variant” as the term is used herein, includes a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

The term “vector”, as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operably linked genes are referred to herein as “expression vectors.”

The term “wild-type”, as used herein, has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

### Genetically Modified Loci

In certain aspects provided herein are genetically modified non-human animals (e.g., mice or rats) comprising: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, provided herein are genetically modified non-human animals (e.g., mice) comprising: (i) a homozygous null mutation in Rag2 gene (e.g., a Rag2 gene knock-out); (ii) a homozygous null mutation in IL2rg gene (e.g., a IL2rg gene knock-out); (iii) a homozygous null mutation in at least one non-human animal Csf2rb gene (e.g., mouse Csf2rb gene and/or mouse Csf2rb2 gene); (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, the genetically modified non-human animal expresses a human GM-CSF protein. In some embodiments, the genetically modified non-human animal expresses a human IL-3 protein. In some embodiments, the genetically modified non-human animal expresses a human or humanized SIRPA protein encoded by a nucleic acid operably linked to a Sirpa promoter. In certain embodiments, the genetically modified non-human animal further expresses one or more human or humanized proteins selected from the group consisting of: a human TPO protein encoded by a nucleic acid operably linked to a TPO promoter; a human M-CSF protein encoded by a nucleic acid operably linked to an M-CSF promoter; and a human or humanized CD47 protein encoded by a nucleic acid operably linked to a CD47 promoter. In certain embodiments, at least one promoter operably linked to a nucleic acid that encodes a human or humanized protein is an endogenous non-human animal promoter. In some embodiments, the genetically modified non-human animal comprises engraftment of human hematopoietic stem cells (HSC).

### Csf2rb Knockout

In certain aspects, genetically modified non-human animals (e.g., mice or rats) provided herein comprise a homozygous null mutation in at least one non-human animal colony stimulating factor 2 receptor subunit beta (Csf2rb) gene.

Csf2rb gene encodes a cell surface receptor subunit that plays a role in immune response and controls the production and differentiation of hematopoietic progenitor cells into lineage-restricted cells. The protein encoded by Csf2rb gene is the common beta chain of the high affinity receptor for IL-3, IL-5, and CSF. It acts by forming a heterodimeric receptor through interaction with different partners such as IL3RA, IL5RA or CSF2RA. In turn, the respective heterodimeric receptors participate in various signaling pathways including interleukin-3, interleukin-5 and granulocyte-macrophage colony-stimulating factor/CSF2 pathways. In unstimulated conditions, the receptor beta subunit interacts constitutively with JAK1 and ligand binding leads to JAK1 stimulation and subsequent activation of the JAK-STAT pathway.

Representative human CSF2RB cDNA and human CSF2RB protein sequences are well-known in the art and are publicly available from the National Center for Biotechnology Information (NCBI). For example, there are at least two human CSF2RB isoforms. CSF2RB isoform 1 (NP_001397756.1) is encoded by transcript NM_001410827.1, whereas CSF2RB isoform 2 (NP_000386.1) is encoded by transcript NM_000395.3. Nucleic acid and polypeptide sequences of Csf2rb orthologs in organisms other than humans are well-known and include, for example, chimpanzee Csf2rb (XM_008975018.5 and XP_008973266.2; XM_003821538.7 and XP_003821586.2; and XM_057300993.1 and XP_057156976.1), Rhesus monkey Csf2rb (XM_015150256.2 and XP_015005742.2; and XM_015150257.2 and XP_015005743.2), cattle Csf2rb (NM_001192664.2 and NP_001179593.2), dog Csf2rb (XM_038679963.1 and XP_038535891.1), rat Csf2rb (NM_133555.1 and NP_598239.1), mouse Csf2rb (NM_001358854.1 and NP_001345783.1; and NM_007780.4 and NP_031806.3), and Chinese hamster Csf2rb (XM_027385455.2 and XP_027241256.2).

In certain aspects, the genetically modified non-human animal comprises a homozygous null mutation in at least one non-human animal Csf2rb gene. A null mutation comprises a deletion, an insertion, and/or a substitution in a gene which leads to a non-functional gene product (e.g., complete absence of the gene product (protein, RNA) at the molecular level, or the expression of a non-functional gene product). In the present disclosure, a null mutation has the same meaning and is used interchangeably with an inactivating mutation. A homozygous null mutation refers to having a null mutation in all alleles. For example, a homozygous null mutation in a mouse or rat Csf2rb gene refers to having a null mutation in two alleles (i.e., two null alleles) for the mouse or rat Csf2rb gene. In some embodiments, a gene with a homozygous null mutation can be also referred to as a gene knockout or a gene deficient/deficiency. For example, in some embodiments, a homozygous null mutation in at least one Csf2rb gene is referred to as a Csf2rb knockout or a Csf2rb-deficiency. Thus, the null mutation in at least one non-human animal Csf2rb gene comprises a deletion, an insertion, and/or a substitution in the at least one non-human animal Csf2rb gene (that leads to a non-functional gene product). In some instances, an endogenous non-human animal Csf2rb locus comprises a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some embodiments, the homozygous null mutation in a non-human animal Csf2rb gene comprises the same null mutation for all the alleles for the non-human animal Csf2rb gene. In some embodiments, the homozygous null mutation in a non-human animal Csf2rb gene comprises different null mutations for different alleles for the non-human animal Csf2rb gene.

In some embodiments, a non-human animal may comprise more than one Csf2rb gene. In some embodiments, a non-human animal may comprise a Csf2rb gene that encodes a common β chain for receptors for GM-CSF, IL3, and IL5, and an additional gene (e.g., a colony stimulating factor 2 receptor subunit beta 2 (Csf2rb2) gene) that structurally resembles the Csf2rb gene and encodes an IL3-specific β chain receptor. For example, mouse has a Csf2rb gene that encodes a common β chain for receptors for GM-CSF, IL3, and IL5, and a Csf2rb2 gene that encodes an IL3-specific β chain receptor, and IL-3 receptor possessing Csf2rb2 and IL-3-specific α chain is sufficient to drive IL-3 signaling in the absence of Csf2rb in mouse. In these embodiments, the genetically modified non-human animal comprising “a homozygous null mutation in at least one non-human animal Csf2rb gene” may comprise a homozygous null mutation in the Csf2rb gene that encodes a common β chain for receptors for GM-CSF, IL3, and IL5, and/or a homozygous null mutation in the additional gene (e.g., the Csf2rb2 gene) that structurally resembles the Csf2rb gene and encodes an IL3-specific β chain receptor.

In some embodiments, the genetically modified non-human animal (e.g., rodent, such as rat or mouse) comprises a homozygous null mutation in the non-human animal (e.g., rodent, such as rat or mouse) Csf2rb gene. In some embodiments, the null mutation in the non-human animal Csf2rb gene is a deletion of the full Csf2rb endogenous coding sequence. In some embodiments, the genetically modified non-human animal is a mouse, and the mouse comprises a homozygous null mutation in the mouse Csf2rb gene and/or a homozygous null mutation in the mouse Csf2rb2 gene. In some embodiments, the genetically modified non-human animal is a mouse comprising a homozygous null mutation in the mouse Csf2rb gene and/or a homozygous null mutation in the mouse Csf2rb2 gene, and the null mutation in the mouse Csf2rb gene is a deletion of the full Csf2rb endogenous coding sequence and/or the null mutation in Csf2rb2 gene is a deletion of the full Csf2rb2 endogenous coding sequence. In some embodiments, the null mutation in at least one Csf2rb gene comprises a deletion of nucleic acid sequence between coordinates chr15: 78,282,507-78,351,090 (GRCm38.p6 assembly). In some embodiments, the non-human animals (e.g., rodents, such as rats or mice) provided herein do not express Csf2rb protein. In some embodiments, the non-human animal is a mouse, and the mouse does not express Csf2rb2 protein. In some embodiments, the non-human animal is a mouse, and the mouse does not express Csf2rb protein or Csf2rb2 protein.

Mouse Csf2rb is located on Chromosome 15, GRCm38.p6, NC_000081.6 (78,325,800-78,351,001), and the mouse Csf2rb coding sequence may be found at Genbank Accession No. NM_001358854.1 (transcript variant 1 which represents the longer transcript) or NM_007780.4 (transcript variant 2 which differs in the 5′ UTR compared to variant 1). Both transcript variants encode the same protein (Genbank Accession No. NP_001345783.1 or NP_031806.3). The mouse Csf2rb locus includes 15 exons, with exons 3-15 being coding exons. As such, in some embodiments, the genetically modified animals provided herein are mice, and one or more of exons 3-15 of the mouse Csf2rb gene are deleted or mutated in the genetically modified mice. In some instances, other aspects of the genomic locus of the mouse Csf2rb gene, e.g., introns, 3′ and/or 5′ untranslated sequence (UTRs) are also deleted or mutated. In some instances, the whole regions of the mouse Csf2rb genomic locus are deleted. In some embodiments, the whole genomic region from the start codon to the stop codon of the mouse Csf2rb gene is deleted.

As discussed above, mouse also has a colony stimulating factor 2 receptor, beta 2, low-affinity (granulocyte-macrophage) (Csf2rb2) gene. Mouse Csf2rb2 gene is located on Chromosome 15, GRCm38.p6, NC_000081.6 (78,282,507-78,305,721, complement), and the mouse Csf2rb2 coding sequence may be found at Genbank Accession No. NM_007781.3 (transcript variant 1 which represents the longer transcript) or NM_001287389.1 (transcript variant 2 which lacks two alternate exons in the coding region compared to variant 1). Transcript variant 1 encodes the longer protein (isoform 1 with Genbank Accession No. NP_031807.1). Transcript variant 2 encodes a shorter protein (isoform 2 with Genbank Accession No. NP_001274318.1) that has the same N- and C-termini compared to isoform 1. The mouse Csf2rb2 locus includes 14 exons, with exons 2-14 being coding exons. As such, in some embodiments, the genetically modified animals provided herein are mice, and one or more of exons 2-14 of the mouse Csf2rb2 gene are deleted or mutated in the genetically modified mice. In some instances, other aspects of the genomic locus of the mouse Csf2rb2 gene, e.g., introns, 3′ and/or 5′ untranslated sequence (UTRs) are also deleted or mutated. In some instances, the whole regions of the mouse Csf2rb2 genomic locus are deleted. In some embodiments, the whole genomic region from the start codon to the stop codon of the mouse Csf2rb2 gene is deleted.

Mouse Csf2rb and Csf2rb2 genes are arranged on Chromosome 15 in a “head-to-head” manner. In some embodiments, the genetically modified mouse comprises a homozygous null mutation in Csf2rb gene. For example, the genetically modified mice may comprise a deletion of mouse genomic sequence between coordinates chr15: 78,325,800-78,351,001 (GRCm38.p6 assembly). In some embodiments, the genetically modified mouse comprises a homozygous null mutation in Csf2rb2 gene. For example, the genetically modified mice may comprise a deletion of mouse genomic sequence between coordinates chr15: 78,282,507-78,305,721, complement (GRCm38.p6 assembly). In some embodiments, the genetically modified mouse comprises a homozygous null mutation in both Csf2rb and Csf2rb2 genes. In some embodiments, mouse genomic sequence of Csf2rb & Csf2rb2 genes from the end of Csf2rb gene to the end of Csf2rb2 gene is deleted, which includes the intergenic region sequence from the start codon ATG of Csf2rb gene to the start codon ATG of Csf2rb2 gene (GRCm38.p6 coordinates chr15: 78,305,722-78,325,799). For example, the genetically modified mice may comprise a deletion of 68 kb of mouse genomic sequence (GRCm38.p6 coordinates chr15: 78,282,507-78,351,090) as illustrated in Example 1.

The deleted, modified or altered Csf2rb gene at the endogenous Csf2rb locus, and/or the deleted, modified or altered Csf2rb2 gene at the endogenous Csf2rb2 locus, can be detected using a variety of methods including, for example, PCR, Western blot, Southern blot, restriction fragment length polymorphism (RFLP), or a gain or loss of allele assay. In some embodiments, the non-human animal is homozygous for the null mutation (e.g., deletion) of the endogenous Csf2rb gene. In some embodiments, the non-human animal is a mouse, and the mouse is homozygous for the null mutation (e.g., deletion) of the endogenous Csf2rb gene and/or the endogenous Csf2rb2 gene.

In some embodiments, the non-human animal (e.g., mouse or rat) comprising a homozygous null mutation in at least one Csf2rb gene, i.e., the Csf2rb deficient non-human animal, is an immunocompromised animal. For example, the Csf2rb deficient non-human animal (e.g., mouse or rat) may include at least one null allele for the Rag2 gene (“recombination activating gene 2”, wherein the coding sequence for the mouse gene may be found at Genbank Accession No. NM_009020.3). In some embodiments, the Csf2rb deficient non-human animal (e.g., mouse or rat) includes two null alleles for Rag2. In other words, the Csf2rb deficient non-human animal (e.g., mouse or rat) is homozygous null for Rag2. In other embodiments, Csf2rb deficient non-human animal (e.g., mouse or rat) includes one or two null alleles for Rag1 gene. In some embodiments, the Csf2rb deficient non-human animal (e.g., mouse or rat) is homozygous null for Rag1. In some embodiments, Csf2rb deficient non-human animal (e.g., mouse or rat) includes (i) one or two null alleles for Rag1 gene; and (ii) one or two null alleles for Rag2 gene. In some embodiments, the Csf2rb deficient non-human animal (e.g., mouse or rat) is homozygous null for both Rag1 and Rag2. In some embodiments, the Csf2rb deficient non-human animal is an immunocompromised mouse comprising two null alleles (i.e., homozygous null) for Rag2. In some embodiments, the Csf2rb deficient non-human animal is an immunocompromised rat comprising two null alleles (i.e., homozygous null) for Rag1 and two null alleles (i.e., homozygous null) for Rag2. As another example, the Csf2rb deficient non-human animal (e.g., mouse or rat) includes at least one null allele for the IL2rg gene (“interleukin 2 receptor, gamma”, also known as the common gamma chain, or γC, wherein the coding sequence for the mouse gene may be found at Genbank Accession No. NM 013563.4). In some embodiments, the Csf2rb deficient non-human animal (e.g., mouse or rat) includes two null alleles for IL2rg. In other words, the Csf2rb deficient non-human animal (e.g., mouse or rat) is homozygous null for IL2rg, i.e., it is IL2rg^{-/-} (or IL2rg^{Y/-} where the IL2rg gene is located on the X chromosome as in mouse). In some embodiments, the Csf2rb deficient non-human animal (e.g., mouse or rat) includes a null allele for both Rag2 and IL2rg, i.e., it is Rag2^{-/-} IL2rg^{-/-} (or Rag2^{-/-} IL2rg^{Y/-} where the IL2rg gene is located on the X chromosome as in mouse or rat). In some embodiments, the Csf2rb deficient non-human animal (e.g., mouse or rat) includes a null allele for both Rag1 and IL2rg. In some embodiments, the Csf2rb deficient non-human animal (e.g., mouse or rat) includes a null allele for Rag1, Rag2, and IL2rg. In some embodiments, the Csf2rb deficient non-human animal is an immunocompromised mouse comprising two null alleles (i.e., homozygous null) for Rag2 and two null alleles (i.e., homozygous null) for IL2rg (or one null allele for male mice). In some embodiments, the Csf2rb deficient non-human animal is an immunocompromised rat comprising two null alleles (i.e., homozygous null) for Rag1, two null alleles (i.e., homozygous null) for Rag2, and two null alleles (i.e., homozygous null) for IL2rg (or one null allele for male rat). Other genetic modifications are also contemplated. For example, the Csf2rb deficient non-human animal (e.g., mouse or rat) may include modifications in other genes associated with the development and/or function of hematopoietic cells and the immune system, e.g., the replacement of one or more other non-human animal genes with nucleic acid sequence(s) encoding human or humanized polypeptides. Such genes include but are not limited to, e.g., SIRPA, CD47, M-CSF, GM-CSF, TPO, and IL-3. Additionally or alternatively, the Csf2rb deficient non-human animal (e.g., mouse or rat) may include modifications in genes associated with the development and/or function of other cells and tissues, e.g., genes associated with human disorders or disease, or genes that, when modified in a non-human animal, e.g., mice, provide for models of human disorders and disease. Introduction of other genetic modifications may be accomplished by either ES cell modification and/or breeding. For example, the Csf2rb deficient (and, optionally, Rag2 and IL2rg deficient) non-human animal (e.g., mouse or rat) may be bred with a non-human animal that comprises one or more other genetic modifications, including but not limited to, e.g., a modification in SIRPA, CD47, M-CSF, GM-CSF, TPO, and IL-3 gene. In some embodiments, all genetic modifications are bred to homozygous in the genetically modified animal described herein.

### Humanized GM-CSF Loci

In some aspects, the genetically modified non-human animals provided herein further express a human GM-CSF protein encoded by a nucleic acid operably linked to a GM-CSF promoter. By a human GM-CSF protein, it is a meant a protein that is human GM-CSF or is substantially identical to human GM-CSF, e.g., it is 80% or more identical, 85% or more identical, 90% or more identical, or 95% or more identical to human GM-CSF, for example, 97%, 98%, or 99% identical to human GM-CSF. A nucleic acid sequence that encodes a human GM-CSF protein is, therefore, a polynucleotide that comprises coding sequence for a human GM-CSF protein, i.e., human GM-CSF or a protein that is substantially identical to human GM-CSF.

GM-CSF is a cytokine crucial for myeloid cell development and function. GM-CSF is not cross-reactive between human and mouse. GM-CSF is highly expressed in the lung and important for lung homeostasis in vivo, as demonstrated by the fact that GM-CSF KO mice develop pulmonary alveolar proteinosis (PAP) which is characterized by protein accumulation in the lung due to defective surfactant clearance. Alveolar macrophages from GM-CSF KO mice have a defect in terminal differentiation, which leads to impaired innate immunity to pathogens in the lung. GM-CSF also stimulates the proliferation of human alveolar macrophages (AM) in vitro. GM-CSF is largely dispensable for steady-state hematopoiesis. In contrast, GM-CSF is required for inflammatory responses such as the production of proinflammatory cytokines by macrophages and the mobilization and recruitment of monocytes. GM-CSF is also essential for protective immunity against a range of pathogens, including M. tuberculosis. In particular, GM-CSF KO mice infected with M. tuberculosis do not develop granulomas, a hallmark of tuberculosis.

Polypeptide sequence for human GM-CSF and the nucleic acid sequence that encodes for human GM-CSF may be found at Genbank Accession Nos. NP_000749.2 and NM_000758.4, respectively. The genomic locus encoding the human GM-CSF protein may be found in the human genome at Chromosome 5; NG_033024.1 (4998-7379). Protein sequence is encoded by exons 1 through 4 at this locus. As such, a nucleic acid sequence comprising coding sequence for human GM-CSF comprises one or more of exons 1-4 of the human GM-CSF gene. In some instances, the nucleic acid sequence also comprises aspects of the genomic locus of the human GM-CSF, e.g., introns, 3′ and/or 5′ untranslated sequence (UTRs). In some instances, the nucleic acid sequence comprises whole regions of the human GM-CSF genomic locus.

In some embodiments, in the genetically modified non-human animals provided herein, the nucleic acid sequence that encodes a human GM-CSF protein is operably linked to one or more regulatory sequences of the non-human animal (e.g., mouse) GM-CSF gene. Non-human animal (e.g., mouse) GM-CSF regulatory sequences are those sequences of the non-human animal (e.g., mouse) GM-CSF genomic locus that regulate non-human animal (e.g., mouse) GM-CSF expression, for example, 5′ regulatory sequences, e.g., the GM-CSF promoter, GM-CSF 5′ untranslated region (UTR), etc.; 3′ regulatory sequences, e.g., the 3′ UTR; and enhancers, etc. For example, mouse GM-CSF is located on chromosome 11, GRCm39, NC_000077.7, at about positions c54140725-54138096, and the mouse GM-CSF coding sequence may be found at Genbank Accession No. NM_009969.4. The regulatory sequences of mouse GM-CSF are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human GM-CSF protein is located at the non-human animal (e.g., mouse) GM-CSF genomic locus, the regulatory sequences operably linked to the human GM-CSF coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human GM-CSF protein is generated by the random integration, or insertion, of human nucleic acid sequence encoding human GM-CSF protein or a fragment thereof, i.e., “human GM-CSF nucleic acid sequence”, or “human GM-CSF sequence”, into the genome of the non-human animal. Typically, in such embodiments, the location of the nucleic acid sequence encoding a human GM-CSF protein in the genome is unknown. In other instances, the genetically modified non-human animal expressing a human GM-CSF protein is generated by the targeted integration, or insertion, of human GM-CSF nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human GM-CSF protein created by targeting human GM-CSF nucleic acid sequence to the non-human animal GM-CSF (e.g., mouse) locus, human GM-CSF nucleic acid sequence may replace some or all of the non-human animal (e.g., mouse) sequence, e.g., exons and/or introns, at the GM-CSF locus. In some such instances, human GM-CSF nucleic acid sequence is integrated into the non-human animal (e.g., mouse) GM-CSF locus such that expression of the human GM-CSF sequence is regulated by the native, or endogenous, regulatory sequences at the non-human animal (e.g., mouse) GM-CSF locus. In other words, the regulatory sequence(s) to which the nucleic acid sequence encoding a human GM-CSF protein is operably linked are the native GM-CSF regulatory sequences at the non-human animal (e.g., mouse) GM-CSF locus.

In some instances, the integration of human GM-CSF sequence does not affect the transcription of the gene into which the human GM-CSF sequence has integrated. For example, if the human GM-CSF sequence integrates into coding sequence as an intein, or the human GM-CSF sequence comprises a 2A peptide, the human GM-CSF sequence will be transcribed and translated simultaneously with the gene into which the human GM-CSF sequence has integrated. In other instances, the integration of the human GM-CSF sequence interrupts the transcription of the gene into which the human GM-CSF sequence has integrated. For example, upon integration of the human GM-CSF sequence by homologous recombination, some or all of the coding sequence at the integration locus may be removed, such that the human GM-CSF sequence is transcribed instead. In some such instances, the integration of human GM-CSF sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human GM-CSF protein comprises one copy of the nucleic acid sequence encoding a human GM-CSF protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human GM-CSF nucleic acid sequence is integrated into the non-human animal (e.g., mouse) GM-CSF locus such that it creates a null allele for non-human animal (e.g., mouse) GM-CSF. In some such embodiments, the humanized GM-CSF mouse may be heterozygous for the nucleic acid sequence encoding, i.e., the humanized GM-CSF mouse comprises one null allele for non-human animal (e.g., mouse) GM-CSF (the allele comprising the nucleic acid sequence) and one endogenous GM-CSF allele (wild type or otherwise). In other instances, the genetically modified non-human animal (e.g., mouse) expressing a human GM-CSF protein comprises two copies of the nucleic acid sequence encoding a human GM-CSF protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human GM-CSF protein comprises two null alleles for the mouse GM-CSF (the allele comprising the nucleic acid sequence).

Although embodiments employing a human GM-CSF gene in a mouse are extensively discussed herein, other non-human animals (e.g., rodents, e.g., rats) that comprise a human GM-CSF gene are also provided.

Human GM-CSF polypeptides, loci encoding human GM-CSF polypeptides and non-human animals expressing human GM-CSF polypeptides are described in WO2011/044050, WO 2014/039782 and WO 2014/071397, each of which is incorporated by reference herein.

### Humanized IL-3 Loci

In some aspects, the genetically modified non-human animals provided herein further express a human IL-3 protein encoded by a nucleic acid operably linked to an IL-3 promoter. By a human IL-3 protein, it is a meant a protein that is human IL-3 or is substantially identical to human IL-3, e.g., it is 80% or more identical, 85% or more identical, 90% or more identical, or 95% or more identical to human IL-3, for example, 97%, 98%, or 99% identical to human IL-3. A nucleic acid sequence that encodes a human IL-3 protein is, therefore, a polynucleotide that comprises coding sequence for a human IL-3 protein, i.e., human IL-3 or a protein that is substantially identical to human IL-3.

Like GM-CSF, IL-3 is a cytokine crucial for myeloid cell development and function. IL-3 is not cross-reactive between human and mouse. IL-3 stimulates early hematopoietic progenitors in vitro, but is dispensable for steady-state hematopoiesis in vivo. However, together with GM-CSF it is required for effective DTH responses in vivo. IL-3 also specifically stimulates the proliferation of alveolar macrophages (AM) in vitro.

Polypeptide sequence for human IL-3 and the nucleic acid sequence that encodes for human IL-3 may be found at Genbank Accession Nos. NP_000579.2 and NM_000588.4. The genomic locus encoding the human IL-3 protein may be found in the human genome at chromosome 5, GRCh38.p14; NC_000005.10 (132060655-132063204). Protein sequence is encoded by exons 1 through 5 at this locus. As such, a nucleic acid sequence comprising coding sequence for human IL-3 comprises one or more of exons 1-5 of the human IL-3 gene. In some instances, the nucleic acid sequence also comprises aspects of the genomic locus of the human IL-3, e.g., introns, 3′ and/or 5′ untranslated sequence (UTRs). In some instances, the nucleic acid sequence comprises whole regions of the human IL-3 genomic locus.

In some embodiments, in the genetically modified non-human animals provided herein, the nucleic acid sequence that encodes a human IL-3 protein is operably linked to one or more regulatory sequences of the non-human animal (e.g., mouse) IL-3 gene. Non-human animal (e.g., mouse) IL-3 regulatory sequences are those sequences of the non-human animal (e.g., mouse) IL-3 genomic locus that regulate non-human animal (e.g., mouse) IL-3 expression, for example, 5′ regulatory sequences, e.g., the IL-3 promoter, IL-3 5′ untranslated region (UTR), etc.; 3′ regulatory sequences, e.g., the 3′ UTR; and enhancers, etc. For example, mouse IL-3 is located on chromosome 11, GRCm39, NC_000077.7, at about positions c54158105-54155911, and the mouse IL-3 coding sequence may be found at Genbank Accession No. NM_010556.4. The regulatory sequences of mouse IL-3 are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human IL-3 protein is located at the non-human animal (e.g., mouse) IL-3 genomic locus, the regulatory sequences operably linked to the human IL-3 coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human IL-3 protein is generated by the random integration, or insertion, of human nucleic acid sequence encoding human IL-3 protein or a fragment thereof, i.e., “human IL-3 nucleic acid sequence”, or “human IL-3 sequence”, into the genome of the non-human animal. Typically, in such embodiments, the location of the nucleic acid sequence encoding a human IL-3 protein in the genome is unknown. In other instances, the genetically modified non-human animal expressing a human IL-3 protein is generated by the targeted integration, or insertion, of human IL-3 nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human IL-3 protein created by targeting human IL-3 nucleic acid sequence to the non-human animal IL-3 (e.g., mouse) locus, human IL-3 nucleic acid sequence may replace some or all of the non-human animal (e.g., mouse) sequence, e.g., exons and/or introns, at the IL-3 locus. In some such instances, human IL-3 nucleic acid sequence is integrated into the non-human animal (e.g., mouse) IL-3 locus such that expression of the human IL-3 sequence is regulated by the native, or endogenous, regulatory sequences at the non-human animal (e.g., mouse) IL-3 locus. In other words, the regulatory sequence(s) to which the nucleic acid sequence encoding a human IL-3 protein is operably linked are the native IL-3 regulatory sequences at the non-human animal (e.g., mouse) IL-3 locus.

In some instances, the integration of human IL-3 sequence does not affect the transcription of the gene into which the human IL-3 sequence has integrated. For example, if the human IL-3 sequence integrates into coding sequence as an intein, or the human IL-3 sequence comprises a 2A peptide, the human IL-3 sequence will be transcribed and translated simultaneously with the gene into which the human IL-3 sequence has integrated. In other instances, the integration of the human IL-3 sequence interrupts the transcription of the gene into which the human IL-3 sequence has integrated. For example, upon integration of the human IL-3 sequence by homologous recombination, some or all of the coding sequence at the integration locus may be removed, such that the human IL-3 sequence is transcribed instead. In some such instances, the integration of human IL-3 sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human IL-3 protein comprises one copy of the nucleic acid sequence encoding a human IL-3 protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human IL-3 nucleic acid sequence is integrated into the non-human animal (e.g., mouse) IL-3 locus such that it creates a null allele for non-human animal (e.g., mouse) IL-3. In some such embodiments, the humanized IL-3 mouse may be heterozygous for the nucleic acid sequence encoding, i.e., the humanized IL-3 mouse comprises one null allele for non-human animal (e.g., mouse) IL-3 (the allele comprising the nucleic acid sequence) and one endogenous IL-3 allele (wild type or otherwise). In other instances, the genetically modified non-human animal (e.g., mouse) expressing a human IL-3 protein comprises two copies of the nucleic acid sequence encoding a human IL-3 protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human IL-3 protein comprises two null alleles for the mouse IL-3 (the allele comprising the nucleic acid sequence).

Although embodiments employing a human IL-3 gene in a mouse are extensively discussed herein, other non-human animals (e.g., rodents, e.g., rats) that comprise a human IL-3 gene are also provided.

Human IL-3 polypeptides, loci encoding human IL-3 polypeptides and non-human animals expressing human IL-3 polypeptides are described in WO2011/044050, WO 2014/039782 and WO 2014/071397, each of which is incorporated by reference herein.

### Immunodeficient Non-Human Animals

As explained above, genetically modified non-human animals comprising Csf2rb deficiency described herein are also immunodeficient because they comprise a deficiency in Rag1 and/or Rag2, and Il2rg genes. Rag1, Rag2, and Il2rg are essential components of the adaptive immune system. When one or more of these genes are mutated in animals, T-cells and B-cells do not mature, and the animals are severely compromised. When these animals are challenged with xenotransplanted cells, they are unable to mount an immune response to the foreign cells.

V(D)J recombination-activating protein 1 (also known as RAG1, recombination-activating 1, recombination activating gene 1, and recombination activating protein 1) is encoded by the Rag1 gene (also known as recombination activating 1). RAG1 is a catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyzes the DNA cleavage activities by introducing a double-strand break between the RSS and the adjacent coding segment. RAG2 is not a catalytic component but is required for all known catalytic activities. RAG1 and RAG2 are essential to the generation of mature B cells and T cells, two types of lymphocytes that are crucial components of the adaptive immune system.

Mouse Rag1 maps to 2 E2; 2 53.88 cM on chromosome 2 (NCBI RefSeq Gene ID 19373; Assembly GRCm39 (GCF_000001635.27); location NC_000068.8 (101468597 . . . 101479877, complement). Reference to the mouse Rag1 gene includes the canonical, wild type form as well as all allelic forms and isoforms. The canonical, wild type mouse RAG1 protein has been assigned UniProt accession number P15919 and NCBI Accession No. NP_033045.2. Reference to mouse RAG1 proteins includes wild type forms as well as all allelic forms and isoforms. An mRNA (cDNA) encoding the canonical isoform is assigned NCBI Accession No. NM_009019.2. Reference to the mouse Rag1 mRNA (cDNA) and coding sequence includes the canonical, wild type forms as well as all allelic forms and isoforms.

Rat Rag1 maps to 3q31 on chromosome 3 (NCBI RefSeq Gene ID 84600; Assembly mRatBN7.2 (GCF_015227675.2); location NC_051338.1 (87917061 . . . 87928158, complement). Reference to the rat Rag1 gene includes the canonical, wild type form as well as all allelic forms and isoforms. The canonical, wild type rat RAG1 protein has been assigned UniProt accession number G3V6K9 and NCBI Accession No. NP_445920.1. Reference to rat RAG1 proteins includes canonical, wild type forms as well as all allelic forms and isoforms. An mRNA (cDNA) encoding the canonical isoform is assigned NCBI Accession No. NM_053468.1. Reference to the rat Rag1 mRNA (cDNA) and coding sequence includes the canonical, wild type forms as well as all allelic forms and isoforms.

An inactivated endogenous Rag1 gene is a Rag1 gene that does not produce a RAG1 protein or does not produce a functional RAG1 protein. The non-human animal (or cell or genome) can comprise the inactivated Rag1 gene in its germline. The non-human animal (or cell or genome) can be homozygous for an inactivating mutation in the Rag1 gene. As one example, an inactivated endogenous Rag1 gene can comprise an insertion, a deletion, or one or more point mutations in the endogenous Rag1 gene resulting in loss of expression of functional RAG1 protein. Some inactivated endogenous Rag1 genes can comprise a deletion or disruption of all of the endogenous Rag1 gene or can comprise a deletion or disruption of a fragment of (i.e., a part of or portion of) the endogenous Rag1 gene. For example, some, most, or all of the coding sequence in the endogenous Rag1 gene can be deleted or disrupted. In one example, a 5′ fragment of the Rag1 gene can be deleted or disrupted (e.g., including the start codon). As one example, an inactivated endogenous Rag1 gene can be one in which the start codon of the endogenous Rag1 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag1 gene can be deleted or disrupted (e.g., including the stop codon). In another example, an internal fragment of the Rag1 gene (i.e., a fragment from the middle of the Rag1 gene) can be deleted or disrupted. In another example, all of the coding sequence in the endogenous Rag1 gene is deleted or disrupted.

V(D)J recombination-activating protein 2 (also known as RAG2, recombination-activating 2, recombination activating gene 2, and recombination activating protein 2) is encoded by the Rag2 gene (also known as recombination activating 2). As mentioned above, RAG1 is a catalytic component of the RAG complex, a multiprotein complex that mediates the DNA cleavage phase during V(D)J recombination. RAG2 is not a catalytic component but is required for all known catalytic activities. RAG1 and RAG2 are essential to the generation of mature B cells and T cells, two types of lymphocyte that are crucial components of the adaptive immune system.

Mouse Rag2 maps to 2 E2; 2 53.87 cM on chromosome 2 (NCBI RefSeq Gene ID 19374; Assembly GRCm39 (GCF_000001635.27); location NC_000068.8 (101455057 . . . 101462873). Reference to the mouse Rag2 gene includes the canonical, wild type form as well as all allelic forms and isoforms. The canonical, wild type mouse RAG2 protein has been assigned UniProt accession number P21784 and NCBI Accession No. NP_033046.1. Reference to mouse RAG2 proteins includes canonical, wild type forms as well as all allelic forms and isoforms. An mRNA (cDNA) encoding the canonical isoform is assigned NCBI Accession No. NM_009020.3. Reference to the mouse Rag2 mRNA (cDNA) and coding sequence includes the canonical, wild type forms as well as all allelic forms and isoforms.

Rat Rag2 maps to 3q31 on chromosome 3 (NCBI RefSeq Gene ID 295953; Assembly mRatBN7.2 (GCF_015227675.2); location NC_051338.1 (87902373 . . . 87910227). Reference to the rat Rag2 gene includes the canonical, wild type form as well as all allelic forms and isoforms. The canonical, wild type rat RAG2 protein has been assigned UniProt accession number G3V6K7 and NCBI Accession No. NP_001093998.1. Reference to rat RAG2 proteins includes canonical, wild type forms as well as all allelic forms and isoforms. An mRNA (cDNA) encoding the canonical isoform is assigned NCBI Accession No. NM_001100528.1. Reference to the rat Rag2 mRNA (cDNA) and coding sequence includes the canonical, wild type forms as well as all allelic forms and isoforms.

An inactivated endogenous Rag2 gene is a Rag2 gene that does not produce a RAG2 protein or does not produce a functional RAG2 protein. The non-human animal (or cell or genome) can comprise the inactivated Rag2 gene in its germline. The non-human animal (or cell or genome) can be homozygous for an inactivating mutation in the Rag2 gene. As one example, an inactivated endogenous Rag2 gene can comprise an insertion, a deletion, or one or more point mutations in the endogenous Rag2 gene resulting in loss of expression of functional RAG2 protein. Some inactivated endogenous Rag2 genes can comprise a deletion or disruption of all of the endogenous Rag2 gene or can comprise a deletion or disruption of a fragment of (i.e., a part of or portion of) the endogenous Rag2 gene. For example, some, most, or all of the coding sequence in the endogenous Rag2 gene can be deleted or disrupted. In one example, a 5′ fragment of the Rag2 gene can be deleted or disrupted (e.g., including the start codon). As one example, an inactivated endogenous Rag2 gene can be one in which the start codon of the endogenous Rag2 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag2 gene can be deleted or disrupted (e.g., including the stop codon). In another example, an internal fragment of the Rag2 gene (i.e., a fragment from the middle of the Rag2 gene) can be deleted or disrupted. In another example, all of the coding sequence in the endogenous Rag2 gene is deleted or disrupted.

Interleukin 2 receptor subunit gamma (also known as interleukin 2 receptor, gamma; interleukin 2 receptor, gamma (severe combined immunodeficiency), isoform CRA_a; cytokine receptor common subunit gamma precursor) is encoded by the Il2rg gene (also known as interleukin 2 receptor subunit gamma or IL2RG). IL2RG is a cytokine receptor subunit that is common to receptor complexes for several different interleukin receptors. IL2RG is located on the surface of immature blood-forming cells in bone marrow. IL2RG partners with other proteins to direct blood-forming cells to form lymphocytes. IL2RG also directs the growth and maturation of T cells, B cells, and natural killer cells. Mutations in Il2rg can cause X-linked severe combined immunodeficiency in which lymphocytes cannot develop normally. A lack of functional mature lymphocytes disrupts the immune system's ability to protect the body from infection.

Mouse Il2rg maps to X D; X 43.9 cM on chromosome X (NCBI RefSeq Gene ID 16186; Assembly GRCm39 (GCF_000001635.27); location NC_000086.8 (100307991 . . . 100311861, complement). Reference to the mouse Il2rg gene includes the canonical, wild type form as well as all allelic forms and isoforms. The canonical, wild type mouse IL2rG protein has been assigned UniProt accession number P34902 and NCBI Accession No. NP_038591.1. Reference to mouse IL2RG proteins includes canonical, wild type forms as well as all allelic forms and isoforms. An mRNA (cDNA) encoding the canonical isoform is assigned NCBI Accession No. NM_013563.4. Reference to the mouse Il2rg mRNA (cDNA) and coding sequence includes the canonical, wild type forms as well as all allelic forms and isoforms.

Rat Il2rg maps to Xq22 on chromosome X (NCBI RefSeq Gene ID 140924; Assembly mRatBN7.2 (GCF_015227675.2); location NC_051356.1 (66395330 . . . 66399026, complement). Reference to the rat Il2rg gene includes the canonical, wild type form as well as all allelic forms and isoforms. The canonical, wild type rat IL2rG protein has been assigned UniProt accession number Q68FU6 and NCBI Accession No. NP_543165.1. Reference to rat IL2RG proteins includes canonical, wild type forms as well as all allelic forms and isoforms. An mRNA (cDNA) encoding the canonical isoform is assigned NCBI Accession No. NM_080889.1. Reference to the rat Il2rg mRNA (cDNA) and coding sequence includes the canonical, wild type forms as well as all allelic forms and isoforms.

An inactivated endogenous Il2rg gene is an Il2rg gene that does not produce a IL2RG protein or does not produce a functional IL2RG protein. The non-human animal (or cell or genome) can comprise the inactivated Il2rg gene in its germline. The non-human animal (or cell or genome) can be homozygous for an inactivating mutation in the Il2rg gene. As one example, an inactivated endogenous Il2rg gene can comprise an insertion, a deletion, or one or more point mutations in the endogenous Il2rg gene resulting in loss of expression of functional IL2RG protein. Some inactivated endogenous Il2rg genes can comprise a deletion or disruption of all of the endogenous Il2rg gene or can comprise a deletion or disruption of a fragment of (i.e., a part of or portion of) the endogenous Il2rg gene. For example, some, most, or all of the coding sequence in the endogenous Il2rg gene can be deleted or disrupted. In one example, a 5′ fragment of the Il2rg gene can be deleted or disrupted (e.g., including the start codon). As one example, an inactivated endogenous Il2rg gene can be one in which the start codon of the endogenous Il2rg gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Il2rg gene can be deleted or disrupted (e.g., including the stop codon). In another example, an internal fragment of the Il2rg gene (i.e., a fragment from the middle of the Il2rg gene) can be deleted or disrupted. In another example, all of the coding sequence in the endogenous Il2rg gene is deleted or disrupted.

### Humanized Sirpa Loci

In certain aspects, the genetically modified non-human animals provided herein further express a human or humanized SIRPA protein encoded by a nucleic acid operably linked to a Sirpa promoter.

Signal regulatory proteins (SIRPs) constitute a family of cell surface glycoproteins which are expressed on lymphocytes, myeloid cells (including macrophages, neutrophils, granulocytes, myeloid dendritic cells, and mast cells) and neurons (e.g., see Barclay and Brown, 2006, Nat Rev Immunol 6, 457-464). The reported SIRP genes include at least SIRPA, SIRP3, SIRPβ, SIRPγ, and SIRP8 and can be categorized by their respective ligands and types of signaling in which they are involved. SIRPA (also referred to as CD172A, SHPS1, P84, MYD-1, BIT and PTPNS1) is expressed on immune cells of the myeloid lineage and functions as an inhibitory receptor via an immunoreceptor tyrosine-based inhibitory motif (ITIM). SIRPA expression has also been observed on neurons. Reported ligands for SIRPA include, most notably, CD47, but also include surfactant proteins A and D. The role of SIRPA, in particular, has been investigated in respect of its inhibitory role in the phagocytosis of host cells by macrophages. For example, CD47 binding to SIRPA on macrophages triggers inhibitory signals that negatively regulates phagocytosis. Alternatively, positive signaling effects mediated through SIRPA binding have been reported (Shultz et al., 1995, J Immunol 154, 180-91). SIRPA has been shown to improve cell engraftment in immunodeficient mice (Strowig et al. Proc Natl Acad Sci USA 2011; 108: 13218-13223).

Polypeptide sequences for wild-type human SIRPA and the nucleic acid sequences that encode wild-type human SIRPA may be found at Genbank Accession Nos. NP_001035111.1 and NM_001040022.1 (isoform 1 and transcript variant 1); NP_001035112.1 and NM_001040023.2 (isoform 1 and transcript variant 2); NP_001317657.1 and NM_001330728.1 (isoform 2 and transcript variant 4); and NP_542970.1 and NM_080792.3 (isoform 1 and transcript variant 3). The SIRPA gene is conserved in at least chimpanzee, Rhesus monkey, dog, cow, mouse, rat, and chicken. The genomic locus encoding the wild-type human SIRPA protein may be found in the human genome at Chromosome 20; NC_000020.11 (1894167-1940592). In some embodiments, human SIRPA protein is encoded by exons 2 through 9 at this locus. As such, in some embodiments, a nucleic acid sequence including coding sequence for human SIRPA includes one or more of exons 2-9 of the human SIRPA gene. In some instances, the nucleic acid sequence also includes aspects of the genomic locus of the human SIRPA, e.g., introns, 3′ and/or 5′ untranslated sequence (UTRs). In some instances, the nucleic acid sequence includes whole regions of the human SIRPA genomic locus. In some instances, the nucleic acid sequence includes exons 2-4 of the human SIRPA genomic locus.

Examples of humanized Sirpa sequences are set forth in Table 1. For protein sequences, signal peptides are underlined, and transmembrane and cytoplasmic sequences are italicized. Representative mouse Sirpa cDNA, mouse Sirpa protein, human SIRPA cDNA, and human SIRPA protein sequences are described in U.S. Pat. No. 11,019,810, which is incorporated by reference herein in its entirety.

TABLE 1
Humanized Sirpa Protein (SEQ ID NO: 1)
MEPAGPAPGRLGPLLLCLLLSASCFCTGVAGEEELQVIQPDKSVLVAAGE
TATLRCTATSLIPVGPIQWFRGAGPGRELIYNQKEGHFPRVTTVSDLTKR
NNMDFSIRIGNITPADAGTYYCVKFRKGSPDDVEFKSGAGTELSVRAKPS
APVVSGPAARATPQHTVSFTCESHGFSPRDITLKWFKNGNELSDFQTNVD
PVGESVSYSIHSTAKVVLTREDVHSQVICEVAHVTLQGDPLRGTANLSET
IRVPPTLEVTQQPVRAENQVNVTCQVRKFYPQRLQLTWLENGNVSRTETA
STVTENKDGTYNWMSWLLVNVSAHRDDVKLTCQVEHDGQPAVSKSHDLKV
SAHPKEQGSNTAADNNATHNWNVFIGVGVACALLVVLLMAALYLLRIKQK
KAKGSTSSTRLHEPEKNAREITQIQDINDINDITYADLNLPKEKKPAPRA
PEPNNHTEYASIETGKVPRPEDTLTYADLDMVHLSRAQPAPKPEPSFSEY
ASVQVQRK

In some embodiments, the non-human animals provided herein express humanized Sirpa proteins on the surface of immune cells (e.g., myeloid cells) of the non-human animals resulting from a genetic modification of an endogenous locus of the non-human animal that encodes a Sirpa protein. Suitable examples described herein include rodents, for example, mice.

A humanized Sirpa gene, in some embodiments, comprises genetic material from a heterologous species (e.g., humans), wherein the humanized Sirpa gene encodes a Sirpa protein that comprises the encoded portion of the genetic material from the heterologous species. In some embodiments, a humanized Sirpa gene of the present disclosure comprises genomic DNA of a heterologous species that corresponds to the extracellular portion of a SIRPA protein that is expressed on the plasma membrane of a cell. Non-human animals, embryos, cells and targeting constructs for making non-human animals, non-human embryos, and cells containing said humanized Sirpa gene are also provided.

In some embodiments, an endogenous non-human animal (e.g., rodent) Sirpa gene is deleted. In some embodiments, an endogenous non-human animal (e.g., rodent) Sirpa gene is altered, wherein a portion of the endogenous non-human animal (e.g., rodent) Sirpa gene is replaced with a heterologous sequence (e.g., a human SIRPA sequence in whole or in part). In some embodiments, all or substantially all of an endogenous non-human animal (e.g., rodent) Sirpa gene is replaced with a heterologous gene (e.g., a human SIRPA gene). In some embodiments, a portion of a heterologous SIRPα gene is inserted into an endogenous non-human Sirpa gene at an endogenous non-human animal (e.g., rodent) Sirpa locus. In some embodiments, the heterologous gene is a human gene. In some embodiments, the modification or humanization is made to one of the two copies of the endogenous non-human animal (e.g., rodent) Sirpa gene, giving rise to a non-human animal which is heterozygous with respect to the humanized Sirpa gene. In other embodiments, a non-human animal is provided that is homozygous for a humanized Sirpa gene. In some embodiments, all of an endogenous non-human animal (e.g., rodent) Sirpa gene is replaced with a portion of heterologous gene (e.g., a portion of human SIRPA gene), such that the genetically modified non-human animal (e.g., rodent) expresses a functional fragment of a full-length human SIRPA polypeptide (e.g., an extracellular domain of a human SIRPA polypeptide).

A non-human animal of the present disclosure contains a human SIRPA gene in whole or in part at an endogenous non-human Sirpa locus. Thus, such non-human animals can be described as having a heterologous SIRP gene. The replaced, inserted or modified SIRPα gene at the endogenous non-human animal (e.g., rodent) Sirpa locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized Sirpa gene.

In various embodiments, a humanized Sirpa gene according to the present disclosure includes a SIRPα gene that has a second, third and fourth exon each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a second, third and fourth exon that appear in a human SIRPA gene.

In various embodiments, a humanized Sirpa gene according to the present disclosure includes a SIRPα gene that has a nucleotide coding sequence (e.g., a cDNA sequence) at least 50% (e.g., 50%, 55%, 60%, 65%, 70%, 75%, 80%, 85%, 90%, 91%, 92%, 93%, 94%, 95%, 96%, 97%, 98%, 99% or more) identical to nucleotides 352-1114 that appear in a human SIRPA cDNA sequence.

In various embodiments, a humanized Sirpa protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an extracellular portion of a human SIRPA protein.

In various embodiments, a humanized Sirpa a protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 28-362 that appear in a human SIRPA protein.

In various embodiments, a humanized Sirpa protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an amino acid sequence of a humanized SIRPA protein that appears in Table 1 (SEQ ID NO:1).

Compositions and methods for making non-human animals that express a humanized Sirpa protein, including specific polymorphic forms or allelic variants (e.g., single amino acid differences), are provided, including compositions and methods for making non-human animals that express such proteins from a human promoter and a human regulatory sequence. In some embodiments, compositions and methods for making non-human animals that express such proteins from an endogenous promoter and an endogenous regulatory sequence are also provided. The methods include inserting the genetic material encoding a human SIRPA protein in whole or in part at a precise location in the genome of a non-human animal that corresponds to an endogenous non-human animal (e.g., rodent) Sirpa gene thereby creating a humanized Sirpa gene that expresses a SIRPA protein that is human in whole or in part. In some embodiments, the methods include inserting genomic DNA corresponding to exons 2-4 of a human SIRPA gene into an endogenous non-human animal (e.g. rodent) Sirpa gene of the non-human animal thereby creating a humanized gene that encodes a Sirpa protein that contains a human portion containing amino acids encoded by the inserted exons.

In various embodiments, a humanized Sirpa gene approach employs a relatively minimal modification of the endogenous gene and results in natural Sirpa-mediated signal transduction in the non-human animal. Thus, in such embodiments, the Sirpa gene modification does not affect other surrounding genes or other endogenous non-human animal (e.g., rodent) Sirp genes. Further, in various embodiments, the modification does not affect the assembly of a functional receptor on the plasma and maintains normal effector functions via binding and subsequent signal transduction through the cytoplasmic portion of the receptor which is unaffected by the modification.

In addition to mice having humanized Sirpa genes as described herein, also provided herein are other genetically modified non-human animals (e.g., rodents, e.g., rats) that comprise humanized Sirpa genes. In some embodiments, such non-human animals comprise a humanized Sirpa gene operably linked to an endogenous Sirpa promoter. In some embodiments, such non-human animals express a humanized Sirpa protein from an endogenous locus, wherein the humanized Sirpa protein comprises amino acid residues 28-362 of a human SIRPA protein.

Humanized Sirpa polypeptides, loci encoding humanized Sirpa polypeptides and non-human animals expressing humanized Sirpa polypeptides are described in U.S. Pat. No. 11,019,810, WO 2014/039782, WO 2014/071397, and WO 2016/168212, each of which is incorporated by reference herein in its entirety.

### Humanized CD47 Loci

In one aspect, non-human animals are provided that are genetically modified to express one or more human proteins from their genome. In certain aspects, the genetically modified non-human animals provided herein further express a human or humanized CD47 protein encoded by a nucleic acid operably linked to a CD47 promoter.

CD47, originally named integrin-associated protein (IAP) for its role in signal transduction from integrins on immune cells, is a transmembrane protein that includes an N-terminal immunoglobulin V (IgV) domain, five transmembrane domains, and a short C-terminal intracytoplasmic tail. The intracytoplasmic tail differs in length according to four alternatively spliced isoforms that have been identified. CD47 (or IAP) was initially described as being expressed on all tissues (isoform 2), neurons (isoform 4) and keratinocytes and macrophages (isoform 1; see Reinhold et al. (1995) J. Cell Sci. 108:3419-3425). In addition to integrins, CD47 is known to interact with several other cell surface proteins such as, for example, thrombospondin and members of the SIRP family. Most notably, CD47 interacts with SIRPA and leads to bidirectional signaling that regulates a variety of cell-to-cell responses such as, for example, inhibition of phagocytosis and T cell activation. Indeed, CD47-SIRPA interaction has come into focus in recent years for its role in providing tumor cells with the capacity to evade immune surveillance. CD47 binding to SIRPA normally provides protection through anti-phagocytic signals (“don't eat me”) for normal cells. However, it has been discovered that tumors also express anti-phagocytic signals, including CD47, to evade destruction by phagocytosis. Interestingly, CD47 is known to be upregulated in several hematologic cancers and contribute to both the growth and dissemination of tumors (Chao et al. (2012) Curr Opin Immunol. 24(2): 225-232).

Polypeptide sequence for wild-type human CD47 and the nucleic acid sequence that encode wild-type human CD47 may be found at Genbank Accession Nos. NP_001369235.1 and NM_001382306.1 (isoform 3 and transcript variant 3); NP_001768.1 and NM_001777.4 (isoform 1 and transcript variant 1); NP_942088.1 and NM_198793.3 (isoform 2 and transcript variant 2); and XP_005247966.1 and XM_005247909.3 (isoform X1 and transcript variant X1). The CD47 gene is conserved in at least chimpanzee, Rhesus monkey, dog, cow, mouse, rat, and chicken. The genomic locus encoding the wild-type human CD47 protein may be found in the human genome at Chromosome 3; NC_000003.12 (c108091031-108043091). In some embodiments, human CD47 protein is encoded by exons 1 through 11 at this locus. As such, in some embodiments, a nucleic acid sequence including coding sequence for human CD47 includes one or more of exons 1-11 of the human CD47 gene. In some instances, the nucleic acid sequence also includes aspects of the genomic locus of the human CD47, e.g., introns, 3′ and/or 5′ untranslated sequence (UTRs). In some instances, the nucleic acid sequence includes whole regions of the human CD47 genomic locus. In some instances, the nucleic acid sequence includes exons 2-7 of the human CD47 genomic locus.

Examples of humanized CD47 sequences are set forth in Table 2. For humanized protein sequences, non-human (e.g., mouse) sequences are indicated in regular font, human sequences are indicated in bold font, and signal peptides are underlined. Representative mouse CD47 cDNA, mouse CD47 protein, human CD47 cDNA, and human CD47 protein sequences are described in U.S. Pat. Pub. No. 2021/0161112 A1, which is incorporated by reference herein in its entirety.

TABLE 2
Humanized CD47 amino acid isoform 1 (SEQ ID NO: 2)
MWPLAAALLLGSCCCGSAQLLFNKTKSVEFTFCNDTVVIPCFVTNMEAQN
TTEVYVKWKFKGRDIYTFDGALNKSTVPTDFSSAKIEVSOLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVE
Humanized CD47 amino acid isoform 2 (SEQ ID NO: 3)
MWPLAAALLLGSCCCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQRTIQ
PPRNR
Humanized CD47 amino acid isoform 3 (SEQ ID NO: 4)
MWPLAAALLLGSCCCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQRTIQ
PPRKAVEEPLNE
Humanized CD47 amino acid isoform 4 (SEQ ID NO: 5)
MWPLAAALLLGSCCCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQRTIQ
PPRKAVEEPLNAFKESKGMMNDE

In some embodiments, the non-human animals provided herein express humanized CD47 proteins on the surface of cells of the non-human animals resulting from a genetic modification of an endogenous locus of the non-human animal that encodes a CD47 protein. Suitable examples described herein include rodents, for example, mice.

A humanized CD47 gene, in some embodiments, comprises genetic material from a heterologous species (e.g., humans), wherein the humanized CD47 gene encodes a CD47 protein that comprises the encoded portion of the genetic material from the heterologous species. In some embodiments, a humanized CD47 gene of the present disclosure comprises genomic DNA of a heterologous species that encodes the extracellular portion of a CD47 protein that is expressed on the plasma membrane of a cell. In some embodiments, a humanized CD47 gene of the present disclosure comprises genomic DNA of a heterologous species that encodes the extracellular portion and the transmembrane portion of a CD47 protein that is expressed on the plasma membrane of a cell. Non-human animals, embryos, cells and targeting constructs for making non-human animals, non-human embryos, and cells containing said humanized CD47 gene are also provided.

In some embodiments, an endogenous CD47 gene is deleted. In some embodiments, an endogenous CD47 gene is altered, wherein a portion of the endogenous CD47 gene is replaced with a heterologous sequence (e.g., a human CD47 sequence, in whole or in part). In some embodiments, all or substantially all of an endogenous CD47 gene is replaced with a heterologous gene (e.g., a human CD47 gene). In some embodiments, a portion of a heterologous CD47 gene is inserted into an endogenous non-human CD47 gene at an endogenous CD47 locus. In some embodiments, the heterologous gene is a human gene. In some embodiments, the modification or humanization is made to one of the two copies of the endogenous CD47 gene, giving rise to a non-human animal that is heterozygous with respect to the humanized CD47 gene. In other embodiments, a non-human animal is provided that is homozygous for a humanized CD47 gene.

In some embodiments, a non-human animal of the present disclosure contains a human CD47 gene, in whole or in part, at an endogenous non-human CD47 locus. Thus, such non-human animals can be described as having a heterologous CD47 gene. The replaced, inserted, modified or altered CD47 gene at the endogenous CD47 locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized CD47 gene. In some embodiments, the non-human animal is homozygous for the humanized CD47 gene.

In various embodiments, a humanized CD47 gene according to the present disclosure includes a CD47 gene that has a second, third, fourth, fifth, sixth and seventh exon each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a second, third, fourth, fifth, sixth and seventh exon that appear in a human CD47 gene.

In various embodiments, a humanized CD47 gene according to the present disclosure includes a CD47 gene that has a first exon and exon(s) downstream of exon 7 (e.g., eighth and ninth exons of isoform 2) each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a respective exon that appears in a mouse CD47 gene.

In various embodiments, a humanized CD47 gene according to the present disclosure includes a CD47 gene that has a 5′ untranslated region and a 3′ untranslated region each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a 5′ untranslated region and a 3′ untranslated region that appear in a mouse CD47 gene.

In various embodiments, a humanized CD47 gene according to the present disclosure includes a CD47 gene that has a nucleotide coding sequence (e.g., a cDNA sequence) at least 50% (e.g., 50%, 55%, 60%, 65%, 70%, 75%, 80%, 85%, 90%, 91%, 92%, 93%, 94%, 95%, 96%, 97%, 98%, 99% or more) identical to a nucleotide coding sequence that appears in a human CD47 nucleotide coding sequence.

In various embodiments, a humanized CD47 protein produced by a non-human animal of the present disclosure has an extracellular portion having an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an extracellular portion of a human CD47 protein.

In various embodiments, a humanized CD47 protein produced by a non-human animal of the present disclosure has an extracellular portion having an amino acid sequence that is identical to amino acid residues 19-141 that appear in a human CD47 protein.

In various embodiments, a humanized CD47 protein produced by a non-human animal of the present disclosure has an N-terminal immunoglobulin V domain having an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an N-terminal immunoglobulin V domain of a human CD47 protein.

In various embodiments, a humanized CD47 protein produced by a non-human animal of the present disclosure has an N-terminal immunoglobulin V domain having an amino acid sequence that is identical to amino acid residues 19-127 that appear in a human CD47 protein.

In various embodiments, a humanized CD47 protein produced by a non-human animal of the present disclosure has an N-terminal immunoglobulin V domain and five transmembrane domains each having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an N-terminal immunoglobulin V domain and five transmembrane domains of a human CD47 protein.

In various embodiments, a humanized CD47 protein produced by a non-human animal of the present disclosure has an intracytoplasmic tail having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an intracytoplasmic tail of a mouse CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 16-292 that appear in a human CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 19-292 that appear in a human CD47 protein.

In various embodiments, a humanized CD47 protein produced by a non-human animal of the present disclosure has an amino acid sequence that is identical to amino acid residues 19-292 (or 16-292) that appear in a human CD47 protein.

In various embodiments, a humanized CD47 protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an amino acid sequence of a humanized CD47 protein that appears in Table 2.

In various embodiments, a humanized CD47 protein produced by a non-human animal of the present disclosure has an amino acid sequence that is identical to an amino acid sequence of a humanized CD47 protein that appears in Table 2.

Compositions and methods for making non-human animals that express a humanized CD47 protein, including specific polymorphic forms, allelic variants (e.g., single amino acid differences) or alternatively spliced isoforms, are provided, including compositions and methods for making non-human animals that express such proteins from a human promoter and a human regulatory sequence. In some embodiments, compositions and methods for making non-human animals that express such proteins from an endogenous promoter and an endogenous regulatory sequence are also provided. The methods include inserting the genetic material encoding a human CD47 protein in whole or in part at a precise location in the genome of a non-human animal that corresponds to an endogenous CD47 gene thereby creating a humanized CD47 gene that expresses a CD47 protein that is human in whole or in part. In some embodiments, the methods include inserting genomic DNA corresponding to exons 2-7 of a human CD47 gene into an endogenous CD47 gene of the non-human animal thereby creating a humanized gene that encodes a CD47 protein that contains a human portion containing amino acids encoded by the inserted exons.

Where appropriate, the coding region of the genetic material or polynucleotide sequence(s) encoding a human CD47 protein in whole or in part may be modified to include codons that are optimized for expression in the non-human animal (e.g., see U.S. Pat. Nos. 5,670,356 and 5,874,304). Codon optimized sequences are synthetic sequences, and preferably encode the identical polypeptide (or a biologically active fragment of a full-length polypeptide which has substantially the same activity as the full-length polypeptide) encoded by the non-codon optimized parent polynucleotide. In some embodiments, the coding region of the genetic material encoding a human CD47 protein, in whole or in part, may include an altered sequence to optimize codon usage for a particular cell type (e.g., a rodent cell). For example, the codons of the genomic DNA corresponding to exons 2-7 of a human CD47 gene to be inserted into an endogenous CD47 gene of a non-human animal (e.g., a rodent) may be optimized for expression in a cell of the non-human animal. Such a sequence may be described as a codon-optimized sequence.

A humanized CD47 gene approach employs a relatively minimal modification of the endogenous gene and results in natural CD47-mediated signal transduction in the non-human animal, in various embodiments, because the genomic sequence of the CD47 is modified in a single fragment and therefore retains normal functionality by including necessary regulatory sequences. Thus, in such embodiments, the CD47 gene modification does not affect other surrounding genes or other endogenous CD47-interacting genes (e.g., thrombospondin, SIRPs, integrins, etc.). Further, in various embodiments, the modification does not affect the assembly of a functional CD47 transmembrane protein on the plasma membrane and maintains normal effector functions via binding and subsequent signal transduction through the cytoplasmic portion of the protein which is unaffected by the modification.

Although embodiments employing a humanized CD47 gene in a mouse (i.e., a mouse with a CD47 gene that encodes a CD47 protein that includes a human portion and a mouse portion) are extensively discussed herein, other non-human animals (e.g., rodents, e.g., rats) that comprise a humanized CD47 gene are also provided. In some embodiments, such non-human animals comprise a humanized CD47 gene operably linked to an endogenous CD47 promoter. In some embodiments, such non-human animals express a humanized CD47 protein from an endogenous locus, wherein the humanized CD47 protein comprises amino acid residues 16-292 (or 19-141 or 19-127) of a human CD47 protein.

Humanized CD47 polypeptides, loci encoding humanized CD47 polypeptides and non-human animals expressing humanized CD47 polypeptides are described in U.S. Pat. Publication No. 2021/0161112, which is incorporated by reference herein.

### Humanized M-CSF Loci

In some aspects, the genetically modified non-human animals provided herein further express a human M-CSF protein encoded by a nucleic acid operably linked to an M-CSF promoter. By a human M-CSF protein, it is a meant a protein that is human M-CSF or is substantially identical to human M-CSF, e.g., it is 80% or more identical, 85% or more identical, 90% or more identical, or 95% or more identical to human M-CSF, for example, 97%, 98%, or 99% identical to human M-CSF. A nucleic acid sequence that encodes a human M-CSF protein is, therefore, a polynucleotide that comprises coding sequence for a human M-CSF protein, i.e., human M-CSF or a protein that is substantially identical to human M-CSF.

M-CSF (also known as CSF-1, for “colony stimulating factor 1”) is a cytokine that controls the production, differentiation, and function of macrophages. Polypeptide sequence for human M-CSF and the nucleic acid sequence that encodes for human M-CSF may be found at Genbank Accession Nos. NP_000748.4 and NM_000757.6 (isoform a and transcript variant 1); NP_757349.2 and NM_172210.3 (isoform b and transcript variant 2); NP_757350.2 and NM_172211.4 (isoform c and transcript variant 3) and NP_757351.2; and NM 172212.3 (isoform a and transcript variant 4). The genomic locus encoding the human M-CSF protein may be found in the human genome at Chromosome 1; NC_000001.11 (109910506-109930992). Protein sequence is encoded by exons 1 through 8 at this locus, while exon 9 comprises untranslated sequence. As such, a nucleic acid sequence comprising coding sequence for human M-CSF comprises one or more of exons 1-8 of the human M-CSF gene. In some instances, the nucleic acid sequence also comprises aspects of the genomic locus of the human M-CSF, e.g., introns, 3′ and/or 5′ untranslated sequence (UTRs). In some instances, the nucleic acid sequence comprises whole regions of the human M-CSF genomic locus. In some instances, the nucleic acid sequence comprises exon 2 of the human M-CSF genomic locus to 633 nucleotides downstream of noncoding exon 9.

In some embodiments, in the genetically modified non-human animals provided herein, the nucleic acid sequence that encodes a human M-CSF protein is operably linked to one or more regulatory sequences of the non-human animal (e.g., mouse) M-CSF gene. Non-human animal (e.g., mouse) M-CSF regulatory sequences are those sequences of the non-human animal (e.g., mouse) M-CSF genomic locus that regulate non-human animal (e.g., mouse) M-CSF expression, for example, 5′ regulatory sequences, e.g., the M-CSF promoter, M-CSF 5′ untranslated region (UTR), etc.; 3′ regulatory sequences, e.g., the 3′UTR; and enhancers, etc. For example, mouse M-CSF is located on chromosome 3, NC_000069.7, at about positions c107668048-107648364, and the mouse M-CSF coding sequence may be found at Genbank Accession Nos. NM_007778.4 (transcript variant 1 encoding isoform 1), NM_001113529.1 (transcript variant 2 encoding isoform 2), and NM 001113530.1 (transcript variant 3 encoding isoform 1). The regulatory sequences of mouse M-CSF are well defined in the art, and may be readily identified using in silico methods, e.g., by referring to the above Genbank Accession Nos. on the UCSC Genome Browser, on the world wide web at genome.ucsc.edu, or by experimental methods as described in the art, e.g., Abboud et al. (2003) Analysis of the Mouse CSF-1 Gene Promoter in a Transgenic Mouse Model. J. Histochemistry and Cytochemistry 51 (7):941-949, the disclosure of which is incorporated herein by reference. In some instances, e.g., when the nucleic acid sequence that encodes a human M-CSF protein is located at the non-human animal (e.g., mouse) M-CSF genomic locus, the regulatory sequences operably linked to the human CSF coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human M-CSF protein is generated by the random integration, or insertion, of human nucleic acid sequence encoding human M-CSF protein or a fragment thereof, i.e., “human M-CSF nucleic acid sequence”, or “human M-CSF sequence”, into the genome of the non-human animal. Typically, in such embodiments, the location of the nucleic acid sequence encoding a human M-CSF protein in the genome is unknown. In other instances, the genetically modified non-human animal expressing a human M-CSF protein is generated by the targeted integration, or insertion, of human M-CSF nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human M-CSF protein created by targeting human M-CSF nucleic acid sequence to the non-human animal M-CSF (e.g., mouse) locus, human M-CSF nucleic acid sequence may replace some or all of the non-human animal (e.g., mouse) sequence, e.g., exons and/or introns, at the M-CSF locus. In some such instances, human M-CSF nucleic acid sequence is integrated into the non-human animal (e.g., mouse) M-CSF locus such that expression of the human M-CSF sequence is regulated by the native, or endogenous, regulatory sequences at the non-human animal (e.g., mouse) M-CSF locus. In other words, the regulatory sequence(s) to which the nucleic acid sequence encoding a human M-CSF protein is operably linked are the native M-CSF regulatory sequences at the non-human animal (e.g., mouse) M-CSF locus.

In some instances, the integration of human M-CSF sequence does not affect the transcription of the gene into which the human M-CSF sequence has integrated. For example, if the human M-CSF sequence integrates into coding sequence as an intein, or the human M-CSF sequence comprises a 2A peptide, the human M-CSF sequence will be transcribed and translated simultaneously with the gene into which the human M-CSF sequence has integrated. In other instances, the integration of the human M-CSF sequence interrupts the transcription of the gene into which the human M-CSF sequence has integrated. For example, upon integration of the human M-CSF sequence by homologous recombination, some or all of the coding sequence at the integration locus may be removed, such that the human M-CSF sequence is transcribed instead. In some such instances, the integration of human M-CSF sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human M-CSF protein comprises one copy of the nucleic acid sequence encoding a human M-CSF protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human M-CSF nucleic acid sequence is integrated into the non-human animal (e.g., mouse) M-CSF locus such that it creates a null allele for non-human animal (e.g., mouse) M-CSF. In some such embodiments, the humanized M-CSF mouse may be heterozygous for the nucleic acid sequence encoding, i.e., the humanized M-CSF mouse comprises one null allele for non-human animal (e.g., mouse) M-CSF (the allele comprising the nucleic acid sequence) and one endogenous M-CSF allele (wild type or otherwise). In other instances, the genetically modified non-human animal (e.g., mouse) expressing a human M-CSF protein comprises two copies of the nucleic acid sequence encoding a human M-CSF protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human M-CSF protein comprises two null alleles for the mouse M-CSF (the allele comprising the nucleic acid sequence).

Although embodiments employing a human M-CSF gene in a mouse are extensively discussed herein, other non-human animals (e.g., rodents, e.g., rats) that comprise a human M-CSF gene are also provided.

Human M-CSF polypeptides, loci encoding human M-CSF polypeptides and non-human animals expressing human M-CSF polypeptides are described in WO 2012/112544, WO 2014/039782, and WO 2014/071397, each of which is incorporated by reference herein.

### Humanized TPO Loci

In some aspects, the genetically modified non-human animals provided herein further express a human TPO protein encoded by a nucleic acid operably linked to a TPO promoter. By a human TPO protein, it is a meant a protein that is human TPO or is substantially identical to human TPO, e.g., it is 80% or more identical, 85% or more identical, 90% or more identical, or 95% or more identical to human TPO, for example, 97%, 98%, or 99% identical to human TPO. A nucleic acid sequence that encodes a human TPO protein is, therefore, a polynucleotide that comprises coding sequence for a human TPO protein, i.e., human TPO or a protein that is substantially identical to human TPO.

Thrombopoietin (TPO) was initially identified as a growth factor that promotes the development of megakaryocytes and platelets. TPO is constitutively produced by the liver and the kidneys and released into the blood circulation. The receptor for TPO, c-Mpl, is expressed by hematopoietic stem and progenitor cells in the bone marrow. C-Mpl is also expressed on circulating platelets. However, the binding of TPO on platelets does not activate any signaling pathway. Thus, thrombocytes act as a sink or scavengers for TPO and via this mechanism contribute to negative regulation of thrombopoiesis. Subsequently, TPO has been recognized for its important function to support the expansion and self-renewal of HSCs. TPO deficiency leads to reduced numbers of HSCs in adult mice, and the presence of TPO is needed to maintain adult HSCs in quiescence. Furthermore, TPO is required to support post-transplantation expansion of HSCs, necessary to replenish the hematopoietic compartment of irradiated hosts. Interestingly, it has been demonstrated that osteoblastic cells involved in forming the HSC niche in the bone marrow produce TPO, critical for HSC function and maintenance.

Polypeptide sequence for human TPO and the nucleic acid sequence that encodes for human TPO may be found at Genbank Accession Nos. NM_000547.6 and NP_000538.3 (transcript variant 1 and isoform a); NM_001206744.2 and NP_001193673.1 (transcript variant 6 and isoform a); NM_001206745.2 and NP_001193674.1 (transcript variant 7 and isoform b); NM_175719.4 and NP_783650.1 (transcript variant 2 and isoform b); NM_175721.3 and NP_783652.1 (transcript variant 4 and isoform d); and NM_175722.3 and NP_783653.1 (transcript variant 5 and isoform e). The genomic locus encoding the human TPO protein may be found in the human genome at Chromosome 2; NG_011581.1 (4999-134265). Protein sequence is encoded by exons 2 through 17 at this locus. As such, a nucleic acid sequence comprising coding sequence for human TPO comprises one or more of exons 2-17 of the human TPO gene. In some instances, the nucleic acid sequence also comprises aspects of the genomic locus of the human TPO, e.g., introns, 3′ and/or 5′ untranslated sequence (UTRs). In some instances, the nucleic acid sequence comprises whole regions of the human TPO genomic locus.

In some embodiments, in the genetically modified non-human animals provided herein, the nucleic acid sequence that encodes a human TPO protein is operably linked to one or more regulatory sequences of the non-human animal (e.g., mouse) TPO gene. Non-human animal (e.g., mouse) TPO regulatory sequences are those sequences of the non-human animal (e.g., mouse) TPO genomic locus that regulate non-human animal (e.g., mouse) TPO expression, for example, 5′ regulatory sequences, e.g., the TPO promoter, TPO 5′ untranslated region (UTR), etc.; 3′ regulatory sequences, e.g., the 3′UTR; and enhancers, etc. For example, mouse TPO is located on chromosome 12, GRCm39, NC_000078.7, at about positions c30182983-30104658, and the mouse TPO coding sequence may be found at Genbank Accession No. NM_009417.3. The regulatory sequences of mouse TPO are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human TPO protein is located at the non-human animal (e.g., mouse) TPO genomic locus, the regulatory sequences operably linked to the human TPO coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human TPO protein is generated by the random integration, or insertion, of human nucleic acid sequence encoding human TPO protein or a fragment thereof, i.e., “human TPO nucleic acid sequence”, or “human TPO sequence”, into the genome of the non-human animal. Typically, in such embodiments, the location of the nucleic acid sequence encoding a human TPO protein in the genome is unknown. In other instances, the genetically modified non-human animal expressing a human TPO protein is generated by the targeted integration, or insertion, of human TPO nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human TPO protein created by targeting human TPO nucleic acid sequence to the non-human animal TPO (e.g., mouse) locus, human TPO nucleic acid sequence may replace some or all of the non-human animal (e.g., mouse) sequence, e.g., exons and/or introns, at the TPO locus. In some such instances, human TPO nucleic acid sequence is integrated into the non-human animal (e.g., mouse) TPO locus such that expression of the human TPO sequence is regulated by the native, or endogenous, regulatory sequences at the non-human animal (e.g., mouse) TPO locus. In other words, the regulatory sequence(s) to which the nucleic acid sequence encoding a human TPO protein is operably linked are the native TPO regulatory sequences at the non-human animal (e.g., mouse) TPO locus.

In some instances, the integration of human TPO sequence does not affect the transcription of the gene into which the human TPO sequence has integrated. For example, if the human TPO sequence integrates into coding sequence as an intein, or the human TPO sequence comprises a 2A peptide, the human TPO sequence will be transcribed and translated simultaneously with the gene into which the human TPO sequence has integrated. In other instances, the integration of the human TPO sequence interrupts the transcription of the gene into which the human TPO sequence has integrated. For example, upon integration of the human TPO sequence by homologous recombination, some or all of the coding sequence at the integration locus may be removed, such that the human TPO sequence is transcribed instead. In some such instances, the integration of human TPO sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human TPO protein comprises one copy of the nucleic acid sequence encoding a human TPO protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human TPO nucleic acid sequence is integrated into the non-human animal (e.g., mouse) TPO locus such that it creates a null allele for non-human animal (e.g., mouse) TPO. In some such embodiments, the humanized TPO mouse may be heterozygous for the nucleic acid sequence encoding, i.e., the humanized TPO mouse comprises one null allele for non-human animal (e.g., mouse) TPO (the allele comprising the nucleic acid sequence) and one endogenous TPO allele (wild type or otherwise). In other instances, the genetically modified non-human animal (e.g., mouse) expressing a human TPO protein comprises two copies of the nucleic acid sequence encoding a human TPO protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human TPO protein comprises two null alleles for the mouse TPO (the allele comprising the nucleic acid sequence).

Although embodiments employing a human TPO gene in a mouse are extensively discussed herein, other non-human animals (e.g., rodents, e.g., rats) that comprise a human TPO gene are also provided.

Human TPO polypeptides, loci encoding human TPO polypeptides and non-human animals expressing human TPO polypeptides are described in WO2011/044050, WO 2014/039782 and WO 2014/071397, each of which is incorporated by reference herein.

### Genetically Modified Non-Human Animals and ES Cells

In certain aspects, provided herein are genetically modified non-human animals (e.g., rodents, such as rats or mice) comprising: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, and optionally one or more of the humanized loci disclosed herein as well as genetically modified non-human animal ES cells useful in the making of such non-human animals.

In certain aspects, provided herein are genetically modified non-human animals (e.g., rodents, such as rats or mice) and non-human animal (e.g., rodent, such as rat or mouse) ES cells comprising in their germline and/or genome: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, and optionally one or more of the engineered loci described herein. In some embodiments, provided herein are genetically modified non-human animals (e.g., mice) and non-human animal (e.g., mouse) ES cells comprising in their germline and/or genome: (i) a homozygous null mutation in Rag2 gene (e.g., a Rag2 gene knock-out); (ii) a homozygous null mutation in IL2rg gene (e.g., a IL2rg gene knock-out); (iii) a homozygous null mutation in at least one non-human animal Csf2rb gene (e.g., mouse Csf2rb gene and/or mouse Csf2rb2 gene); (iv) a nucleic acid sequence that encodes a human GM-CSF and is operably linked to a GM-CSF promoter; and (v) a nucleic acid sequence that encodes a human IL-3 and is operably linked to a IL-3 promoter, and optionally one or more of the engineered loci described herein. For example, in some embodiments, the non-human animal or ES cell comprises in its germline and/or genome a humanized Sirpa locus provided herein. In some embodiments, the non-human animal or ES cell comprises in their germline and/or genome a CD47 locus provided herein. In certain embodiments, the non-human animal or ES cell comprises in their germline and/or genome an M-CSF locus provided herein. In certain embodiments, the non-human animal or ES cell comprises in their germline and/or genome a TPO locus provided herein. In some embodiments, the non-human animal or ES cell is heterozygous for one or more of the loci, e.g., genetically engineered loci, provided herein. In some embodiments, the non-human animal or ES cell is homozygous for one or more of the loci, e.g., genetically engineered loci, provided herein.

In some embodiments, the non-human animal can be any non-human animal. In some embodiments, the non-human animal is a vertebrate. In some embodiments, the non-human animal is a mammal. In some embodiments, the genetically modified non-human animal described herein may be selected from a group consisting of a mouse, rat, rabbit, pig, bovine (e.g., cow, bull, buffalo), deer, sheep, goat, llama, chicken, cat, dog, ferret, primate (e.g., marmoset, rhesus monkey). For non-human animals where suitable genetically modifiable ES cells are not readily available, other methods can be employed to make a non-human animal comprising the genetic modifications described herein. Such methods include, for example, modifying a non-ES cell genome (e.g., a fibroblast or an induced pluripotent cell) and employing nuclear transfer to transfer the modified genome to a suitable cell, such as an oocyte, and gestating the modified cell (e.g., the modified oocyte) in a non-human animal under suitable conditions to form an embryo.

In some embodiments, the non-human animal is a mammal. In some embodiments, the non-human animal is a small mammal, e.g., of the superfamily Dipodoidea or Muroidea. In some embodiments, the non-human animal is a rodent. In certain embodiments, the rodent is a mouse, a rat or a hamster. In some embodiments, the rodent is selected from the superfamily Muroidea. In some embodiments, the non-human animal is from a family selected from Calomyscidae (e.g., mouse-like hamsters), Cricetidae (e.g., hamster, New World rats and mice, voles), Muridae (e.g., true mice and rats, gerbils, spiny mice, crested rats), Nesomyidae (e.g., climbing mice, rock mice, white-tailed rats, Malagasy rats and mice), Platacanthomyidae (e.g., spiny dormice), and Spalacidae (e.g., mole rates, bamboo rats, and zokors). In some embodiments, the rodent is selected from a true mouse or rat (family Muridae), a gerbil, a spiny mouse, and a crested rat. In some embodiments, the mouse is from a member of the family Muridae. In some embodiments, the non-human animal is a rodent. In some embodiments, the rodent is selected from a mouse and a rat. In some embodiments, the non-human animal is a mouse.

In some embodiments, the non-human animal is a mouse of a C57BL strain. In some embodiments, the C57BL strain is selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the non-human animal is a mouse of a 129 strain. In some embodiments, the 129 strain is selected from the group consisting of a strain that is 129P1, 129P2, 129P3, 129X1, 12951 (e.g., 12951/SV, 129S1/SvIm), 12952, 12954, 12955, 12959/SvEvH, 12956 (129/SvEvTac), 12957, 12958, 129T1, 129T2. In some embodiments, the genetically modified mouse is a mix of a 129 strain and a C57BL strain. In some embodiments, the mouse is a mix of 129 strains and/or a mix of C57BL/6 strains. In some embodiments, the 129 strain of the mix is a 12956 (129/SvEvTac) strain. In some embodiments, the mouse is a BALB strain (e.g., BALB/c). In some embodiments, the mouse is a mix of a BALB strain and another strain (e.g., a C57BL strain and/or a 129 strain). In some embodiments, the non-human animals provided herein can be a mouse derived from any combination of the aforementioned strains.

In some embodiments, the non-human animal provided herein is a rat. In some embodiments, the rat is selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

In certain embodiments, the genetically modified non-human animals or ES cells comprise in their genome and/or germline multiple loci provided herein, such as multiple genetically engineered loci provided herein. For example, in some embodiments, the non-human animal or ES cell comprises in its germline and/or genome: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, the non-human animal or ES cell comprises in its germline and/or genome (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, and a humanized Sirpa locus provided herein. In some embodiments, the non-human animal or ES cell comprises in its germline and/or genome (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, a human or humanized Sirpa locus provided herein, a humanized M-CSF locus provided herein, a humanized TPO locus provided herein, and a humanized CD47 locus provided herein. In some embodiments the non-human animal or ES cell comprises in its germline and/or genome (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, a humanized Sirpa locus provided herein, and optionally one or more humanized loci selected from the group consisting of a humanized CD47 locus provided herein, a humanized M-CSF locus provided herein, a humanized TPO locus provided herein, and any combinations thereof.

In certain aspects, the genetically modified non-human animal (e.g., rodent, such as rat or mouse) does not express Csf2rb polypeptide. In certain aspects, the genetically modified non-human animal (e.g., mouse) does not express Csf2rb2 polypeptide. In certain aspects, the genetically modified non-human animal (e.g., mouse) does not express Csf2rb and Csf2rb2 polypeptide. In certain aspects, the genetically modified non-human animal expresses one or more of the human or humanized polypeptides encoded by the humanized loci provided herein. For example, in some embodiments, the non-human animal expresses a human GM-CSF polypeptide and/or a human IL-3 polypeptide. In certain embodiments, the non-human animal expresses a human or humanized Sirpa polypeptide. In certain embodiments, the non-human animal expresses a human or humanized CD47 polypeptide. In certain embodiments, the non-human animal expresses a human or humanized M-CSF polypeptide. In certain embodiments, the non-human animal expresses a human or humanized TPO polypeptide.

The genetically modified non-human animals and ES cells can be generated using any appropriate method known in the art. For example, such genetically modified non-human animal ES cells can be generated using VELOCIGENE® technology, which is described in U.S. Pat. Nos. 6,586,251, 6,596,541, 7,105,348, and Valenzuela et al. (2003) “High-throughput engineering of the mouse genome coupled with high-resolution expression analysis” Nat. Biotech. 21(6): 652-659, each of which is hereby incorporated by reference. Modifications can also be made using a genome targeted nuclease system, such as a CRISPR/Cas system, a transcription activator-like effector nuclease (TALEN) system or a zinc finger nuclease (ZFN) system. In some embodiments, modifications are made using a CRISPR/Cas system, as described, for example, in U.S. patent application Ser. Nos. 14/314,866, 14/515,503, 14/747,461 and 14/731,914, each of which is incorporated by reference. Genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties). Examples of methods of making such genetically modified non-human animals and ES cells are also provided herein in Example 1.

ES cells described herein can then be used to generate a non-human animal using methods known in the art. For example, the mouse non-human animal ES cells described herein can be used to generate genetically modified mice using the VELOCIMOUSE® method, as described in U.S. Pat. No. 7,294,754 and Poueymirou et al., Nature Biotech 25:91-99 (2007), each of which is hereby incorporated by reference. Resulting mice can be bread to homozygosity.

### Methods of Making Genetically Modified Non-Human Animals and ES Cells

In certain aspects, provided herein are methods of making non-human animals (e.g., a mouse or a rat) and ES cells that comprise one or more of the genetically modified loci provided here. For example, in some embodiments, provided herein are methods of making non-human animals (e.g., a mouse or a rat) and ES cells that comprise (i) a homozygous null mutation in Rag1 and/or Rag2 gene (e.g., a Rag1 and/or Rag2 gene knock-out; (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter. In some embodiments, provided herein are methods of making non-human animals (e.g., a mouse or a rat) and ES cells that comprise (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in at least one non-human animal colony stimulating factor 2 receptor subunit beta (Csf2rb) gene; (iv) a nucleic acid sequence that encodes a human GM-CSF and is operably linked to a GM-CSF promoter; and (v) a nucleic acid sequence that encodes a human IL-3 and is operably linked to a IL-3 promoter, and further comprise a humanized Sirpa locus provided herein. In some embodiments, provided herein are methods of making non-human animals (e.g., a mouse or a rat) and ES cells that further comprise a humanized CD47 locus provided herein, a humanized M-CSF locus provided herein, and/or a humanized TPO locus provided herein. The methods of making genetically modified non-human animals and ES cells provided herein are described in the description, examples, and/or figures herein. The generation of a non-human animal comprising a null mutation in at least one non-human animal Csf2rb gene may be accomplished using any convenient method for making genetically modified animals, e.g., as known in the art or as described herein in Example 1.

The generation of a non-human animal comprising a nucleic acid sequence that encodes a human or humanized protein (e.g., hSIRPA, hCD47, hM-CSF, hGM-CSF, hTPO, or hIL-3) may be accomplished using any convenient method for making genetically modified animals, e.g., as known in the art or as described herein.

For example, a nucleic acid encoding the human or humanized protein (e.g., hSIRPA, hCD47, hM-CSF, hGM-CSF, hTPO, or hIL-3) may be incorporated into a recombinant vector in a form suitable for insertion into the genome of the host cell and expression of the human protein in a non-human host cell. In various embodiments, the recombinant vector may include the one or more regulatory sequences operably linked to the nucleic acid encoding the human protein in a manner which allows for transcription of the nucleic acid into mRNA and translation of the mRNA into the human protein, as described above. It will be understood that the design of the vector may depend on such factors as the choice of the host cell to be transfected and/or the amount of human protein to be expressed.

Any of various methods may then be used to introduce the human nucleic acid sequence into an animal cell to produce a genetically modified animal that expresses the human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006, Genetically Engineered Mice Handbook, CRC Press), Hofker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol Biol, Humana Press), Meyer et al. (2010, Proc. Nat. Acad. Sci. USA 107: 15022-15026), and Gibson (2004, A Primer Of Genome Science 2nd ed. Sunderland, Massachusetts: Sinauer), U.S. Pat. No. 6,586,251, Rathinam et al. (2011, Blood 118:3119-28), Willinger et al, (2011, Proc Natl Acad Sci USA, 108:2390-2395), Rongvaux et al, (2011, Proc Natl Acad Sci USA, 108:2378-83) and Valenzuela et al. (2003, Nat Biot 21:652-659).

For example, the subject genetically modified animals can be created by introducing the nucleic acid encoding the human protein into an oocyte, e.g., by microinjection, and allowing the oocyte to develop in a female foster animal. In preferred embodiments, the expression is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

As another example, the construct comprising the nucleic acid sequence encoding the human protein may be transfected into stem cells (e.g., ES cells or iPS cells) using well-known methods, such as electroporation, calcium-phosphate precipitation, lipofection, etc. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be introduced into preimplantation embryos. For a detailed description of methods known in the art useful for the compositions and methods of the invention, see Nagy et al, (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

Additionally, as described in some of the Examples below, a nucleic acid construct may be constructed using VELOCIGENE® genetic engineering technology (see, e.g., Valenzuela et al. (2003) High throughput engineering of the mouse genome coupled with high-resolution expression analysis, Nature Biotech. 21(6): 652-59 and U.S. Pat. No. 6,586,251), introduced into stem cells (e.g., ES cells), and correctly targeted clones determined using loss-of-allele and gain-of-allele assays (Valenzuela et al, supra); correctly targeted ES cells may be used as donor ES cells for introduction into an 8-cell stage mouse embryo using the VELOCIMOUSE® method (see, e.g., U.S. Pat. No. 7,294,754 and Poueymirou et al. 2007, F0 generation mice that are essentially fully derived from the donor gene-targeted ES cells allowing immediate phenotypic analyses Nature Biotech. 25(1):91-99). In addition, genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties).

In some embodiments, genetically modified founder animals can be bred to additional animals carrying one or more genetic modifications. For example, Csf2rb-deficient non-human animals provided herein can further be bred to other genetically modified non-human animals carrying other genetic modifications, which include introduction of either completely human or humanized genes, e.g., hSirpa knock-in mice, hM-CSF knock-in mice, hCD47 knock-in mice, hIL-3 knock-in mice, hGM-CSF knock-in mice, hTPO knock-in mice, and the like, or be bred to knockout animals, e.g., a non-human animal that is deficient for one or more proteins, e.g., does not express one or more of its genes, e.g., a Rag1-deficient animal, a Rag2-deficient animal, or an Il2rg-deficient animal.

In another embodiment, stem cells, e.g., ES cells, may be generated such that they comprise several genetic modifications, e.g., humanizations or gene deletions described herein, and such stem cells may be introduced into an embryo to generate genetically modified animals with several genetic modifications. One such embodiment is described in the Examples.

As discussed above, in some embodiments, the genetically modified non-human animal is an immunodeficient animal. Genetically modified non-human animals that are immunodeficient and comprise one or more human or humanized proteins, e.g., hSIRPA, hIL-3, hGM-CSF, hM-CSF, hCD47, and/or hTPO, may be generated using any convenient method for the generation of genetically modified animals, e.g., as known in the art or as described herein. For example, the generation of the genetically modified immunodeficient animal can be achieved by introduction of the nucleic acid encoding the human protein into an oocyte or stem cells comprising a mutant SCID gene allele or Rag1 and/or Rag2 and Il2rg null alleles that, when homozygous, will result in immunodeficiency as described in greater detail above and in the working examples herein. Mice are then generated with the modified oocyte or ES cells using, e.g., methods described herein and known in the art, and mated to produce the immunodeficient mice comprising the desired genetic modification. As another example, genetically modified non-human animals can be generated in an immunocompetent background, and crossed to an animal comprising a mutant gene allele that, when hemizygous or homozygous, will result in immunodeficiency, and the progeny mated to create an immunodeficient animal expressing the at least one human protein of interest.

In some embodiments, the genetically modified mouse is treated so as to eliminate endogenous hematopoietic cells that may exist in the mouse. In one embodiment, the treatment comprises irradiating the genetically modified mouse. In a specific embodiment, newborn genetically modified mouse pups are irradiated sublethally. In a specific embodiment, newborn pups are irradiated 2×200 cGy with a four-hour interval.

Various embodiments of the invention provide genetically modified animals that include a human nucleic acid in substantially all of their cells, as well as genetically modified animals that include a human nucleic acid in some, but not all their cells. In some instances, e.g., targeted recombination, one copy of the human nucleic acid will be integrated into the genome of the genetically modified animals. In other instances, e.g., random integration, multiple copies, adjacent or distant to one another, of the human nucleic acid may be integrated into the genome of the genetically modified animals.

Thus, in some embodiments, the subject genetically modified non-human animal may be an immunodeficient animal comprising a genome that includes a nucleic acid encoding a human polypeptide operably linked to the corresponding non-human animal promoter, wherein the animal expresses the encoded human polypeptide. In other words, the subject genetically modified immunodeficient non-human animal comprises a genome that comprises a nucleic acid encoding at least one human polypeptide, wherein the nucleic acid is operably linked to the corresponding non-human promoter and a polyadenylation signal, and wherein the animal expresses the encoded human polypeptide.

Additional methods of generating genetically modified non-human animals comprising a genome that includes a nucleic acid encoding one or more human proteins, e.g., hSIRPA, hIL-3, hGM-CSF, hM-CSF, hCD47, and/or hTPO, are described in US Pat. No. U.S. Ser. No. 11/019,810, US Pat. Publ. No. US 2021/0161112, WO 2011/044050, WO 2012/112544, WO 2014/039782, and WO 2014/071397, each of which each of which is incorporated by reference herein.

### Engraftment

In some embodiments, the subject genetically modified non-human animal is also immunodeficient. “Immunodeficient” includes deficiencies in one or more aspects of an animal's native, or endogenous, immune system, e.g., the animal is deficient for one or more types of functioning host immune cells, e.g., deficient for non-human B cell number and/or function, non-human T cell number and/or function, non-human NK cell number and/or function, etc.

One method to achieve immunodeficiency in the subject animals is sublethal irradiation. Alternatively or in addition, immunodeficiency may be achieved by any one of a number of gene mutations known in the art, any of which may be bred either alone or in combination into the subject genetically modified non-human animals of the present disclosure or which may be used as the source of stem cells into which the genetic modifications of the subject disclosure may be introduced. Non-limiting examples include X-linked SCID, associated with IL2RG gene mutations and characterized by the lymphocyte phenotype T(−) B(+) NK(−); autosomal recessive SCID associated with Jak3 gene mutations and characterized by the lymphocyte phenotype T(−) B(+) NK(−); ADA gene mutations characterized by the lymphocyte phenotype T(−) B(−) NK(−); IL-7R alpha-chain mutations characterized by the lymphocyte phenotype T(−) B(+) NK(+); CD3 delta or epsilon mutations characterized by the lymphocyte phenotype T(−) B(+) NK(+); RAG1 and RAG2 mutations characterized by the lymphocyte phenotype T(−) B(−) NK(+); Artemis gene mutations characterized by the lymphocyte phenotype T(−) B(−) NK(+), CD45 gene mutations characterized by the lymphocyte phenotype T(−) B(+) NK(+); and Prkdcscld mutations characterized by the lymphocyte phenotype T(−), B(−). As such, in some embodiments, the genetically modified immunodeficient non-human animal has one or more deficiencies selected from an IL2 receptor gamma chain deficiency, a Jak3 deficiency, an ADA deficiency, an IL7R deficiency, a CD3 deficiency, a RAG1 and/or RAG2 deficiency, an Artemis deficiency, a CD45 deficiency, and a Prkdc deficiency. In one embodiment, the immunodeficiency is achieved by gene mutation or deletion in Rag1 and/or Rag2 and Il2rg genes. These and other animal models of immunodeficiency will be known to the ordinarily skilled artisan, any of which may be used to generate immunodeficient animals of the present disclosure.

In some embodiments, genetically modified non-human animals in accordance with the invention find use as recipients of human hematopoietic cells that are capable of developing human immune cells from engrafted human hematopoietic cells. As such, in some aspects of the invention, the subject genetically modified animal is a genetically modified, immunodeficient, non-human animal that is engrafted with human hematopoietic cells.

Any source of human hematopoietic cells, human hematopoietic stem cells (HSCs) and/or hematopoietic stem progenitor cells (HSPC) as known in the art or described herein may be transplanted into the genetically modified immunodeficient non-human animals of the present disclosure. One suitable source of human hematopoietic cells known in the art is human umbilical cord blood cells, in particular CD34-positive (CD34+) cells. Another source of human hematopoietic cells is human fetal liver. Another source is human bone marrow. Also encompassed are induced pluripotent stem cells (iPSC) and induced hematopoietic stem cells (iHSC) produced by the de-differentiation of somatic cells, e.g., by methods known in the art. Methods for the transplantation of human cells into non-human animals are well-described in the art and elsewhere herein, any of which may be employed by the ordinarily skilled artisan to arrive at the subject genetically modified engrafted non-human animals.

Cell populations of particular interest include those that comprise hematopoietic stem or progenitor cells, which will contribute to or reconstitute the hematopoietic system of the genetically modified non-human animals, for example, peripheral blood leukocytes, fetal liver cells, fetal bone, fetal thymus, fetal lymph nodes, vascularized skin, artery segments, and purified hematopoietic stem cells, e.g., mobilized HSCs or cord blood HSCs.

Cells may be from any mammalian species, e.g., murine, rodent, canine, feline, equine, bovine, ovine, primate, human, etc. In one embodiment, the cells are human cells. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines are maintained for fewer than 10 passages in vitro.

If the cells are primary cells, they may be harvested from an individual by any convenient method. For example, cells, e.g., blood cells, e.g., leukocytes, may be harvested by apheresis, leukocytapheresis, density gradient separation, etc. As another example, cells, e.g., skin, muscle, bone marrow, spleen, liver, pancreas, lung, intestine, stomach tissue, etc. may be harvested by biopsy. An appropriate solution may be used for dispersion or suspension of the harvested cells. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

In some instances, a heterogeneous population of cells will be transplanted into the genetically modified non-human animals. In other instances, a population of cells that is enriched for a particular type of cell, e.g., a progenitor cell, e.g., a hematopoietic progenitor cell, will be engrafted into the genetically modified non-human animals. Enrichment of a cell population of interest may be by any convenient separation technique. For example, the cells of interest may be enriched by culturing methods. In such culturing methods, particular growth factors and nutrients are typically added to a culture that promote the survival and/or proliferation of one cell population over others. Other culture conditions that affect survival and/or proliferation include growth on adherent or non-adherent substrates, culturing for particular lengths of time, etc. Such culture conditions are well known in the art. As another example, cells of interest may be enriched for by separation the cells of interest from the initial population by affinity separation techniques. Techniques for affinity separation may include magnetic separation using magnetic beads coated with an affinity reagent, affinity chromatography, “panning” with an affinity reagent attached to a solid matrix, e.g., plate, cytotoxic agents joined to an affinity reagent or used in conjunction with an affinity reagent, e.g., complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the cells of interest.

For example, using affinity separation techniques, cells that are not the cells of interest for transplantation may be depleted from the population by contacting the population with affinity reagents that specifically recognize and selectively bind markers that are not expressed on the cells of interest. For example, to enrich for a population of hematopoietic progenitor cells, one might deplete cells expressing mature hematopoietic cell markers. Additionally or alternatively, positive selection and separation may be performed using by contacting the population with affinity reagents that specifically recognize and selectively bind markers associated with hematopoietic progenitor cells, e.g., CD34, CD133, etc. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody, i.e., an antibody that is specific for CD34, CD133, etc. In some embodiments, the affinity reagent may be a specific receptor or ligand for CD34, CD133, etc., e.g., a peptide ligand and receptor; effector and receptor molecules, a T-cell receptor specific for CD34, CD133, etc., and the like. In some embodiments, multiple affinity reagents specific for the marker of interest may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g., phycoerythrin and allophycocyanins, fluorescein and Texas red. Frequently each antibody is labeled with a different fluorochrome, to permit independent sorting for each marker.

The initial population of cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent are selected for by any convenient affinity separation technique, e.g., as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for a cell type of interest, e.g., hematopoietic cells, are achieved in this manner. The cells will be about 70%, about 75%, about 80%, about 85% about 90% or more of the cell composition, about 95% or more of the enriched cell composition, and will preferably be about 95% or more of the enriched cell composition. In other words, the composition will be a substantially pure composition of cells of interest.

The cells to be transplanted into the genetically modified non-human animals, be they a heterogeneous population of cells or an enriched population of cells, may be transplanted immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. Additionally or alternatively, the cells may be cultured in vitro under various culture conditions. Culture medium may be liquid or semi-solid, e.g., containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g., penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The cells may be genetically modified prior to transplanting to the genetically modified non-human animals, e.g., to provide a selectable or traceable marker, to induce a genetic defect in the cells (e.g., for disease modeling), to repair of a genetic defect or ectopically express a gene in the cells (e.g., to determine if such modifications will impact the course of a disease), etc. Cells may be genetically modified by transfection or transduction with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the cells, various techniques may be employed. The genome of the cells may be restricted and used with or without amplification. The polymerase chain reaction; gel electrophoresis; restriction analysis; Southern, Northern, and Western blots; sequencing; or the like, may all be employed. General methods in molecular and cellular biochemistry for these and other purposes disclosed in this application can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The cells may be transplanted in the genetically modified non-human animals by any convenient method, including, for example, intra-hepatic injection, tail-vein injection, retro-orbital injection, and the like. Typically, about 0.5×10⁵-2×10⁶ pluripotent or progenitor cells are transplanted, e.g., about 1×10⁵-1×10⁶ cells, or about 2×10⁵-5×10⁵ cells. In some instances, the mouse is sublethally irradiated prior to transplanting the human cells. In other words, the mouse is exposed to a sublethal dose of radiation, e.g., as described in the examples section below and as well-known in the art. The engrafted genetically modified non-human animal is then maintained under laboratory animal husbandry conditions for at least 1 week, e.g., 1 week or more, or two weeks or more, sometimes 4 weeks or more, and in some instances 6 weeks or more, to allow sufficient reconstitution of the immune system with the engrafted cells.

In some embodiments, the transplanted human hematopoietic cells give rise in the genetically modified non-human animal to one or more engrafted human cells selected from a human CD34-positive cell, a human hematopoietic stem cell, a human hematopoietic cell, a myeloid progenitor cell, an erythroid progenitor cell, a myeloid cell, a dendritic cell, a monocyte, a neutrophil, a mast cell, an erythrocyte, and a combination thereof. In one embodiment, the human cell is present at 4 months, 5 months, 6 months, 7 months, 8 months, 9 months, 10 months, 11 months, or 12 months after engraftment. In a specific embodiment, the human cells comprise human neutrophils.

In some embodiments, the transplanted human hematopoietic cells give rise in the genetically modified non-human animal to an engrafted human hemato-lymphoid system that comprises human hematopoietic stem and progenitor cells, human myeloid progenitor cells, human myeloid cells, human dendritic cells, human monocytes, human granulocytes, human neutrophils, human mast cells, human erythrocytes, human thymocytes, human T cells, human B cells, and human platelets. In one embodiment, the human hemato-lymphoid system is present at 4 months, 5 months, 6 months, 7 months, 8 months, 9 months, 10 months, 11 months, or 12 months after engraftment. In a specific embodiment, the human hemato-lymphoid system comprises human neutrophils.

In some aspects, the genetically modified non-human animal provided herein is engrafted with human hematopoietic cells comprising a disease-specific mutation.

### Nonlimiting Applications of Genetically Modified Engrafted Mice

The genetically modified non-human animals of the present disclosure find many uses in the art. For example, engrafted genetically modified animals of the present disclosure are useful for studying the function of human neutrophils. As another example, engrafted genetically modified mice of the present disclosure provide a useful system for screening candidate agents for desired activities in vivo, for example, to identify agents that are able to modulate (i.e., promote or suppress) the function of human neutrophils, e.g., in a healthy or a diseased state (e.g., as cancerous cells, during pathogen infection, etc.). For example, engrafted genetically modified mice of the present disclosure can be used to identify novel therapeutics. As another example, engrafted genetically modified animals of the present disclosure provide a useful system for predicting the responsiveness of an individual to a disease therapy, e.g., by providing an in vivo platform for screening the responsiveness of an individual's immune system to an agent, e.g., a therapeutic agent, to predict the responsiveness of an individual to that agent.

In some embodiments, engrafted genetically modified animals of the present disclosure with increased human neutrophils can be useful for testing any therapeutic that targets neutrophils as effectors (e.g., an agent targeting FcαR, an IgA antibody therapy, etc.) as well as disease modeling in which human neutrophils are an important component.

In some embodiments, engrafted genetically modified animals of the present disclosure with increased human neutrophils represent superior models for testing therapeutics specifically targeting neutrophils. For example, neutrophils are believed to be the drivers of Granulomatosis with polyangiitis (GPA), a debilitating vasculitis disease resulting from autoantibodies directed against neutrophil-specific proteins such as PR3 and MPO (ANCA antibodies). An engrafted genetically modified non-human animals of the present disclosure find use in modeling of such anti-neutrophil cytoplasmic autoimmune (ANCA) vasculitis. Human immune system models of the ANCA vasculitis are useful in both research, e.g., to better understand the ANCA vasculitis in humans, and in drug discovery, e.g., to identify candidate agents that treat the ANCA vasculitis.

A human immune system model with increased human neutrophils is an ideal platform to model this disease in vivo as well as test potential treatments. As another example, tumor infiltrating neutrophils have been linked to tumor progression and poor prognosis. Since human immune system models are frequently used for immune-oncology studies and therapeutic testing, a model with increased human neutrophils is superior for testing the role of neutrophils in tumor progression and can unravel novel ways to mitigate neutrophil-mediated tumor progression. As another example, because neutrophils are considered the first line of defense of the immune system against various bacterial and fungal pathogens, a human immune system model with increased human neutrophils represent a superior platform to test pathogen treatment options that target neutrophils.

Under certain stimulation, neutrophils release their internal chromatin in a form of “NETs” that can ensnare invasive pathogens. This process, referred to as NETosis, is also believed to be a major source of autoantigen in a variety of autoimmune diseases that manifest anti-nuclear antibodies, e.g. lupus. It is speculated that overt stimulation of NETosis, coupled with lack of clearance of these neutrophil nets, contributes to autoantibody development. Thus, a HIS model with increased human neutrophils would allow the in vivo study of human neutrophil NETosis, as well as model neutrophil NETosis in autoimmune diseases with the potential to test novel therapeutics that mitigate NETosis in autoimmunity.

Engrafted genetically modified animals of the present disclosure find use in screening candidate agents to identify those that will prevent (e.g., vaccines) or treat bacterial or fungal infections, treat ANCA vasculitis, or mitigate neutrophil-mediated tumor progression. The terms “treatment”, “treating” and the like are used herein to generally include obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein include any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The terms “individual,” “subject,” “host,” and “patient,” are used interchangeably herein and include any mammalian subject for whom diagnosis, treatment, or therapy is desired, particularly humans.

In screening assays for biologically active agents, a human hematopoietic cell-engrafted genetically modified non-human animal of the present disclosure, is contacted with a candidate agent of interest and the effect of the candidate agent is assessed by monitoring one or more output parameters. These output parameters may be reflective of the viability of the human neutrophils, e.g., the total number of human neutrophils; or of the apoptotic state of the human neutrophils, e.g., the amount of cell blebbing, the amount of phosphatidylserine on the human neutrophil surface, and the like, by methods that are well known in the art. Alternatively or additionally, the output parameters may be reflective of the function of the human neutrophils, e.g., phagocytosis, cytokine production, or activation of human neutrophils, by methods that are well known in the art. Alternatively or additionally, the output parameters may be reflective of the effect of the agent on preventing or treating a disease (e.g., bacterial or fungal infection, GPA, ANCA vasculitis, or tumor progression) in the human hematopoietic cell-engrafted genetically modified non-human animal of the present disclosure.

Candidate agents for screening or methods of the present disclosure may include, for examples, organic molecules (e.g., small molecule inhibitors), nucleic acids (e.g., RNA interfering agents, oligonucleotides, or nucleic acids that encode polypeptides), peptides, peptidomimetic inhibitors, aptamers, antibodies, intrabodies, etc. The “RNA interfering agent” used herein may be a small interfering RNA (siRNA), CRISPR RNA (crRNA), CRISPR guide RNA (gRNA), a small hairpin RNA (shRNA), a microRNA (miRNA), or a piwi-interacting RNA (piRNA).

Candidate agents are screened for biological activity by administering the agent to at least one and usually a plurality of samples, sometimes in conjunction with samples lacking the agent. The change in parameters in response to the agent is measured, and the result evaluated by comparison to reference samples, e.g., in the presence and absence of the agent, obtained with other agents, etc. In instances in which a screen is being performed to identify candidate agents that will prevent, mitigate or reverse the effects of a pathogen, the screen is typically performed in the presence of the pathogenic agent, where the pathogenic agent is added at the time most appropriate to the results to be determined. For example, in cases in which the protective/preventative ability of the candidate agent is tested, the candidate agent may be added before the pathogen, simultaneously with the pathogen, or subsequent to infection by the pathogen. As another example, in cases in which the ability of the candidate agent to reverse the effects of a pathogen is tested, the candidate agent may be added subsequent to infection with the pathogen. As mentioned above, in some instances, the “sample” is a genetically modified non-human animal that has been engrafted with cells, e.g., the candidate agent is provided to a genetically modified non-human animal that has been engrafted with human hematopoietic cells. In some instances, the “sample” is the human hematopoietic cells to be engrafted, i.e., the candidate agent is provided to cells, e.g., human neutrophils, etc., prior to engraftment into the immunodeficient genetically modified animal.

If the candidate agent is to be administered directly to the engrafted genetically modified animal, the agent may be administered by any of a number of well-known methods in the art for the administration of peptides, small molecules and nucleic acids to mice. For example, the agent may be administered orally, mucosally, topically, intradermally, or by injection, e.g., intraperitoneal, subcutaneous, intramuscular, or intravenous injection, and the like. The agent may be administered in a buffer, or it may be incorporated into any of a variety of formulations, e.g., by combination with appropriate pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. The agent may be systemic after administration or may be localized by the use of regional administration, intramural administration, or use of an implant that acts to retain the active dose at the site of implantation. The active agent may be formulated for immediate activity or it may be formulated for sustained release. If the agent(s) are provided to cells prior to engraftment, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

An analysis of the response of cells in the engrafted genetically modified animal to the candidate agent may be performed at any time following treatment with the agent. For example, the cells may be analyzed 1, 2, or 3 days, sometimes 4, 5, or 6 days, sometimes 8, 9, or 10 days, sometimes 14 days, sometimes 21 days, sometimes 28 days, sometimes 1 month or more after contact with the candidate agent, e.g., 2 months, 4 months, 6 months or more. In some embodiments, the analysis comprises analysis at multiple time points. The selection of the time point(s) for analysis will be based upon the type of analysis to be performed, as will be readily understood by the ordinarily skilled artisan.

The analysis may comprise measuring any of the parameters described herein or known in the art for measuring cell viability, cell proliferation, cell identity, cell morphology, and cell function, particularly as they may pertain to cells of the immune cells. For example, flow cytometry may be used to determine the total number of hematopoietic cells or the number of cells of a particular hematopoietic cell type. Histochemistry or immunohistochemistry may be performed to determine the apoptotic state of the cells, e.g., terminal deoxynucleotidyl transferase dUTP nick end labeling (TUNEL) to measure DNA fragmentation, or immunohistochemistry to detect Annexin V binding to phosphatidylserine on the cell surface. Flow cytometry may also be employed to assess the proportions of differentiated cells and differentiated cell types, e.g., to determine the ability of hematopoietic cells to survive and/or differentiate in the presence of agent. ELISAs, Westerns, and Northern blots may be performed to determine the levels of cytokines, chemokines, immunoglobulins, etc. expressed in the engrafted genetically modified mice, e.g., to assess the function of the engrafted cells, to assess the survival and/or function of human neutrophils, etc. In vivo assays to test the function of immune cells, as well as assays relevant to particular diseases or disorders of interest such as anemia, e.g., sickle cell anemia, etc. may also be performed. See, e.g., Current Protocols in Immunology (Richard Coico, ed. John Wiley & Sons, Inc. 2012) and Immunology Methods Manual (I. Lefkovits ed., Academic Press 1997), the disclosures of which are incorporated herein by reference.

Other examples of uses for the subject mice are provided elsewhere herein. Additional applications of the genetically modified and engrafted mice described in this disclosure will be apparent to those skilled in the art upon reading this disclosure.

### Additional Embodiments

In embodiment 1, provided herein is a genetically modified non-human animal, comprising: (1) a homozygous null mutation in Rag2 gene; (2) a homozygous null mutation in IL2rg gene; (3) a homozygous null mutation in at least one non-human animal colony stimulating factor 2 receptor subunit beta (Csf2rb) gene; (4) a nucleic acid sequence that encodes a human GM-CSF and is operably linked to a GM-CSF promoter; and (5) a nucleic acid sequence that encodes a human IL-3 and is operably linked to a IL-3 promoter.

In embodiment 2, provided herein is a genetically modified non-human animal of embodiment 1 comprising a homozygous null mutation in Rag1 gene.

In embodiment 3, provided herein is a genetically modified non-human animal of embodiment 1 or 2, comprising a homozygous null mutation in the non-human animal Csf2rb gene.

In embodiment 4, provided herein is a genetically modified non-human animal of any one of embodiments 1-3, wherein the null mutation in the non-human animal Csf2rb gene is a deletion of the full Csf2rb endogenous coding sequence.

In embodiment 5, provided herein is a genetically modified non-human animal of any one of embodiments 1-4, wherein the genetically modified non-human animal is a mouse, and the mouse comprises a homozygous null mutation in colony stimulating factor 2 receptor subunit beta 2 (Csf2rb2) gene.

In embodiment 6, provided herein is a genetically modified non-human animal of embodiment 5, wherein the null mutation in Csf2rb2 gene is a deletion of the full Csf2rb2 endogenous coding sequence.

In embodiment 7, provided herein is a genetically modified non-human animal of embodiment 5 or 6, wherein the mouse comprises a homozygous null mutation in Csf2rb gene and a homozygous null mutation in Csf2rb2 gene.

In embodiment 8, provided herein is a genetically modified non-human animal of embodiment 7, wherein the null mutation in Csf2rb gene is a deletion of the full Csf2rb endogenous coding sequence, and the null mutation in Csf2rb2 gene is a deletion of the full Csf2rb2 endogenous coding sequence.

In embodiment 9, provided herein is a genetically modified non-human animal of embodiment 8, wherein the mouse comprises a homozygous deletion of nucleic acid sequence between coordinates chr15: 78,282,507-78,351,090 (GRCm38.p6 assembly).

In embodiment 10, provided herein is a genetically modified non-human animal of any one of embodiments 1-9, wherein the genetically modified non-human animal expresses a human GM-CSF protein.

In embodiment 11, provided herein is a genetically modified non-human animal of any one of embodiments 1-10, wherein the genetically modified non-human animal expresses a human IL-3 protein.

In embodiment 12, provided herein is a genetically modified non-human animal of any one of embodiments 1-11, wherein the genetically modified non-human animal expresses a human or humanized SIRPA polypeptide encoded by a nucleic acid operably linked to a Sirpa promoter.

In embodiment 13, provided herein is a genetically modified non-human animal of embodiment 12, wherein the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In embodiment 14, provided herein is a genetically modified non-human animal of embodiment 13, wherein the Sirpa gene comprises exons 2-4 of a human SIRPA gene.

In embodiment 15, provided herein is a genetically modified non-human animal of embodiment 13 or 14, wherein the genetically modified non-human animal expresses a Sirpa polypeptide comprising an extracellular portion of a human SIRPA polypeptide and an intracellular portion of a non-human animal Sirpa polypeptide.

In embodiment 16, provided herein is a genetically modified non-human animal of any one of embodiments 13-15, wherein the non-human animal Sirpa polypeptide is an endogenous non-human animal Sirpa polypeptide, and/or the non-human animal Sirpa gene is an endogenous non-human animal gene.

In embodiment 17, provided herein is a genetically modified non-human animal of embodiment 12, wherein the genetically modified non-human animal expresses a human SIRPA polypeptide encoded by a nucleic acid operably linked to a Sirpa promoter.

In embodiment 18, provided herein is a genetically modified non-human animal of any one of embodiments 12-17, wherein the genetically modified non-human animal further expresses one or more human or humanized proteins selected from the group consisting of: a human TPO protein encoded by a nucleic acid operably linked to a TPO promoter; a human M-CSF protein encoded by a nucleic acid operably linked to an M-CSF promoter; and a human or humanized CD47 protein encoded by a nucleic acid operably linked to a CD47 promoter.

In embodiment 19, provided herein is a genetically modified non-human animal of any one of embodiments 12-18, wherein at least one promoter operably linked to a nucleic acid that encodes a human or humanized protein is an endogenous non-human animal promoter.

In embodiment 20, provided herein is a genetically modified non-human animal of embodiment 19, wherein all promoters operably linked to the nucleic acids that encode the human or humanized proteins are endogenous non-human animal promoters.

In embodiment 21, provided herein is a genetically modified non-human animal of embodiment 19 or 20, wherein the endogenous non-human animal promoter is le at the corresponding non-human animal gene locus.

In embodiment 22, provided herein is a genetically modified non-human animal of any one of embodiments 12-21, comprising a null mutation in at least one corresponding non-human animal gene at the corresponding non-human animal gene locus.

In embodiment 23, provided herein is a genetically modified non-human animal of any one of embodiments 12-22, wherein the genetically modified non-human animal is heterozygous for at least one allele comprising the nucleic acid sequence that encodes the human or humanized protein.

In embodiment 24, provided herein is a genetically modified non-human animal of any one of embodiments 12-22, wherein the genetically modified non-human animal is homozygous for at least one allele comprising the nucleic acid sequence that encodes the human or humanized protein.

In embodiment 25, provided herein is a genetically modified non-human animal of any one of embodiments 12-24, wherein the genetically modified non-human animal expresses a human M-CSF protein encoded by a nucleic acid operably linked to an M-CSF promoter.

In embodiment 26, provided herein is a genetically modified non-human animal of any one of embodiments 12-25, wherein the genetically modified non-human animal expresses a human or humanized CD47 protein encoded by a nucleic acid operably linked to a CD47 promoter.

In embodiment 27, provided herein is a genetically modified non-human animal of embodiment 26, wherein the genetically modified non-human animal expresses a humanized CD47 protein, and the humanized CD47 protein comprises an extracellular portion of a human CD47 protein and an intracellular portion of an endogenous non-human animal CD47 protein.

In embodiment 28, provided herein is a genetically modified non-human animal of any one of embodiments 12-27, wherein the genetically modified non-human animal expresses a human TPO protein encoded by a nucleic acid operably linked to a TPO promoter.

In embodiment 29, provided herein is a genetically modified non-human animal of any one of embodiments 12-28, wherein the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; (iii) a human or humanized CD47 protein encoded by a nucleic acid operably linked to a CD47 promoter; and (iv) a human TPO protein encoded by a nucleic acid operably linked to a TPO promoter.

In embodiment 30, provided herein is a genetically modified non-human animal of any one of embodiments 1-29, further comprising an engraftment of human hematopoietic cells.

In embodiment 31, provided herein is a genetically modified non-human animal of embodiment 30, wherein the human hematopoietic cells comprise one or more cells selected from the group consisting of a human CD34-positive cell, a human hematopoietic stem cell, a human hematopoietic progenitor cell, a human neutrophil precursor cell, and a human neutrophil.

In embodiment 32, provided herein is a genetically modified non-human animal of embodiment 30 or 31, wherein the genetically modified non-human animal comprises human neutrophils.

In embodiment 33, provided herein is a genetically modified non-human animal of embodiment 32, wherein anti-neutrophil cytoplasmic autoimmune (ANCA) vasculitis is established in the genetically modified non-human animal.

In embodiment 34, provided herein is a genetically modified non-human animal of embodiment 32 or 33, wherein Granulomatosis with polyangiitis (GPA) is established in the genetically modified non-human animal.

In embodiment 35, provided herein is a genetically modified non-human animal of embodiment 32, wherein a tumor is established in the genetically modified non-human animal.

In embodiment 36, provided herein is a genetically modified non-human animal of embodiment 32, wherein the genetically modified non-human animal is infected by a bacterial or fungal pathogen.

In embodiment 37, provided herein is a genetically modified non-human animal of embodiment 32, wherein human neutrophil NETosis is established in the genetically modified non-human animal.

In embodiment 38, provided herein is a genetically modified non-human animal of embodiment 33, wherein the human neutrophil NETosis provides an autoantigen in the genetically modified non-human animal.

In embodiment 39, provided herein is a genetically modified non-human animal of embodiment 37 or 38, wherein the genetically modified non-human animal has an autoimmune disease that involves the human neutrophil NETosis.

In embodiment 40, provided herein is a genetically modified non-human animal of any one of embodiments 1-39, wherein the genetically modified non-human animal is a mammal.

In embodiment 41, provided herein is a genetically modified non-human animal of embodiment 40, wherein the mammal is a rodent, such as a rat or a mouse.

In embodiment 42, provided herein is a genetically modified non-human animal of embodiment 41, wherein the rodent is a mouse.

In embodiment 43, provided herein is a method for identifying an agent that treats anti-neutrophil cytoplasmic autoimmune (ANCA) vasculitis, the method comprising: administering the agent to a genetically modified non-human animal of embodiment 33; and determining whether the agent treats ANCA vasculitis in the non-human animal.

In embodiment 44, provided herein is a method for identifying an agent that treats Granulomatosis with polyangiitis (GPA), the method comprising: administering the agent to a genetically modified non-human animal of embodiment 34; and determining whether the agent treats GPA in the non-human animal.

In embodiment 45, provided herein is a method for identifying an agent that mitigates tumor progression, the method comprising: administering the agent to a genetically modified non-human animal of embodiment 35; and determining whether the agent mitigates tumor progression in the non-human animal.

In embodiment 46, provided herein is a method for identifying an agent that inhibits an infection by a bacterial or fungal pathogen, the method comprising: administering the agent to a genetically modified non-human animal of embodiment 36, and determining whether the agent reduces the amount of the pathogen and/or inhibits the activity of the pathogen in the pathogen-infected non-human animal.

In embodiment 47, provided herein is a method for identifying an agent that mitigates NETosis, the method comprising: administering the agent to a genetically modified non-human animal of any one of embodiments 37-39; and determining whether the agent mitigates NETosis in the non-human animal.

In embodiment 48, provided herein is a method for identifying an agent that treats an autoimmune disease that involves NETosis, the method comprising: administering the agent to a genetically modified non-human animal of embodiment 39; and determining whether the agent treats the autoimmune disease in the non-human animal.

In embodiment 49, provided herein is a method of any one of embodiments 43-48, wherein the agent targets human neutrophils.

In embodiment 50, provided herein is a method for assessing therapeutic efficacy of a drug candidate to modulate a function of a human neutrophil, the method comprising: administering the drug candidate to a genetically modified non-human animal of embodiment 32; and determining whether the drug candidate modulates the function of the human neutrophil in the genetically modified non-human animal.

In embodiment 51, provided herein is a method of embodiment 50, wherein the drug candidate is an agent targeting FcαR.

In embodiment 52, provided herein is a method of embodiment 50 or 51, wherein the function of the human neutrophil cell is selected from a group consisting of phagocytosis, cytokine production, and activation.

In embodiment 53, provided herein is a genetically modified non-human animal cell, comprising: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to an IL-3 promoter.

In embodiment 54, provided herein is a genetically modified non-human animal cell of embodiment 53 comprising a homozygous null mutation in Rag1 gene.

In embodiment 55, provided herein is a genetically modified non-human animal cell of embodiment 53 or 54, comprising a homozygous null mutation in the non-human animal Csf2rb gene.

In embodiment 56, provided herein is a genetically modified non-human animal cell of any one of embodiments 53-55, wherein the null mutation in the non-human animal Csf2rb gene is a deletion of the full Csf2rb endogenous coding sequence.

In embodiment 57, provided herein is a genetically modified non-human animal cell of any one of embodiments 53-56, wherein the genetically modified non-human animal cell is a mouse cell, and the mouse cell comprises a homozygous null mutation in Csf2rb2 gene.

In embodiment 58, provided herein is a genetically modified non-human animal cell of embodiment 57, wherein the null mutation in Csf2rb2 gene is a deletion of the full Csf2rb2 endogenous coding sequence.

In embodiment 59, provided herein is a genetically modified non-human animal cell of embodiment 57 or 58, wherein the mouse cell comprises a homozygous null mutation in Csf2rb gene and a homozygous null mutation in Csf2rb2 gene.

In embodiment 60, provided herein is a genetically modified non-human animal cell of embodiment 59, wherein the null mutation in Csf2rb gene is a deletion of the full Csf2rb endogenous coding sequence, and the null mutation in Csf2rb2 gene is a deletion of the full Csf2rb2 endogenous coding sequence.

In embodiment 61, provided herein is a genetically modified non-human animal cell of embodiment 60, wherein the mouse cell comprises a homozygous deletion of nucleic acid sequence between coordinates chr15: 78,282,507-78,351,090 (GRCm38.p6 assembly).

In embodiment 62, provided herein is a genetically modified non-human animal cell of any one of embodiments 53-61, wherein the genetically modified non-human animal cell expresses a human GM-CSF protein.

In embodiment 63, provided herein is a genetically modified non-human animal cell of any one of embodiments 53-62, wherein the genetically modified non-human animal cell expresses a human IL-3 protein.

In embodiment 64, provided herein is a genetically modified non-human animal cell of any one of embodiments 53-63, wherein the genetically modified non-human animal cell further comprises a nucleic acid that encodes a human or humanized SIRPA polypeptide, and wherein the nucleic acid is operably linked to a Sirpa promoter.

In embodiment 65, provided herein is a genetically modified non-human animal cell of embodiment 64, wherein the genetically modified non-human animal cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In embodiment 66, provided herein is a genetically modified non-human animal cell of embodiment 65, wherein the Sirpa gene comprises exons 2-4 of a human SIRPA gene.

In embodiment 67, provided herein is a genetically modified non-human animal cell of embodiment 65 or 66, wherein the genetically modified non-human animal cell expresses a Sirpa polypeptide comprising an extracellular portion of a human SIRPA polypeptide and an intracellular portion of a non-human animal Sirpa polypeptide.

In embodiment 68, provided herein is a genetically modified non-human animal cell of any one of embodiments 65-67, wherein the non-human animal Sirpa polypeptide is an endogenous non-human animal Sirpa polypeptide, and/or the non-human animal Sirpa gene is an endogenous non-human animal gene.

In embodiment 69, provided herein is a genetically modified non-human animal cell of embodiment 64, wherein the genetically modified non-human animal cell expresses a human SIRPA polypeptide.

In embodiment 70, provided herein is a genetically modified non-human animal cell of any one of embodiments 64-69, wherein the genetically modified non-human animal cell further comprises one or more nucleic acids selected from the group consisting of: (1) a nucleic acid that encodes a human TPO protein and is operably linked to a TPO promoter; (2) a nucleic acid that encodes a human M-CSF protein and is operably linked to an M-CSF promoter; and (3) a nucleic acid that encodes a human or humanized CD47 protein and is operably linked to a CD47 promoter.

In embodiment 71, provided herein is a genetically modified non-human animal cell of any one of embodiments 64-70, wherein at least one promoter operably linked to a nucleic acid that encodes a human or humanized protein is an endogenous non-human animal promoter.

In embodiment 72, provided herein is a genetically modified non-human animal cell of embodiment 71, wherein all promoters operably linked to the nucleic acids that encode the human or humanized proteins are endogenous non-human animal promoters.

In embodiment 73, provided herein is a genetically modified non-human animal cell of embodiment 71 or 72, wherein the endogenous non-human animal promoter is at the corresponding non-human animal gene locus.

In embodiment 74, provided herein is a genetically modified non-human animal cell of any one of embodiments 64-73, comprising a null mutation in at least one corresponding non-human animal gene at the corresponding non-human animal gene locus.

In embodiment 75, provided herein is a genetically modified non-human animal cell of any one of embodiments 64-74, wherein the genetically modified non-human animal cell is heterozygous for at least one allele comprising the nucleic acid sequence that encodes the human or humanized protein.

In embodiment 76, provided herein is a genetically modified non-human animal cell of any one of embodiments 64-74, wherein the genetically modified non-human animal cell is homozygous for at least one allele comprising the nucleic acid sequence that encodes the human or humanized protein.

In embodiment 77, provided herein is a genetically modified non-human animal cell of any one of embodiments 64-76, wherein the genetically modified non-human animal cell expresses a human M-CSF protein.

In embodiment 78, provided herein is a genetically modified non-human animal cell of any one of embodiments 64-77, wherein the genetically modified non-human animal cell expresses a human or humanized CD47 protein.

In embodiment 79, provided herein is a genetically modified non-human animal cell of embodiment 78, wherein the genetically modified non-human animal cell expresses a humanized CD47 protein, and the humanized CD47 protein comprises an extracellular portion of a human CD47 protein and an intracellular portion of an endogenous non-human animal CD47 protein.

In embodiment 80, provided herein is a genetically modified non-human animal cell of any one of embodiments 64-79, wherein the genetically modified non-human animal cell expresses a human TPO protein.

In embodiment 81, provided herein is a genetically modified non-human animal cell of any one of embodiments 64-80, wherein the genetically modified non-human animal cell expresses: (i) a human or humanized SIRPA protein; (ii) a human M-CSF protein; and (iii) a human or humanized CD47 protein; and (iv) a human TPO protein.

In embodiment 82, provided herein is a genetically modified non-human animal cell of any one of embodiments 53-81, wherein the genetically modified non-human animal cell is a mammalian cell.

In embodiment 83, provided herein is a genetically modified non-human animal cell of embodiment 82, wherein the mammalian cell is a rodent cell, such as a rat cell or a mouse cell.

In embodiment 84, provided herein is a genetically modified non-human animal cell of embodiment 83, wherein the rodent cell is a mouse cell.

In embodiment 85, provided herein is a genetically modified non-human animal cell of any one of embodiments 53-61, 64-66, 68, 70-76, and 82-84, wherein the genetically modified non-human animal cell is a non-human animal embryonic stem (ES) cell.

In embodiment 86, provided herein is a genetically modified non-human animal embryonic stem cell, comprising: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to an IL-3 promoter.

In embodiment 87, provided herein is a genetically modified non-human animal embryonic stem cell of embodiment 86 comprising a homozygous null mutation in Rag1 gene.

In embodiment 88, provided herein is a genetically modified non-human animal embryonic stem cell of embodiment 86 or 87, comprising a homozygous null mutation in the non-human animal Csf2rb gene.

In embodiment 89, provided herein is a genetically modified non-human animal embryonic stem cell of any one of embodiments 86-88, wherein the null mutation in the non-human animal Csf2rb gene is a deletion of the full Csf2rb endogenous coding sequence.

In embodiment 90, provided herein is a genetically modified non-human animal embryonic stem cell of any one of embodiments 86-89, wherein the genetically modified non-human animal embryonic stem cell is a mouse embryonic stem cell, and the mouse embryonic stem cell comprises a homozygous null mutation in Csf2rb2 gene.

In embodiment 91, provided herein is a genetically modified non-human animal embryonic stem cell of embodiment 90, wherein the null mutation in Csf2rb2 gene is a deletion of the full Csf2rb2 endogenous coding sequence.

In embodiment 92, provided herein is a genetically modified non-human animal embryonic stem cell of embodiment 90 or 91, wherein the mouse embryonic stem cell comprises a homozygous null mutation in Csf2rb gene and a homozygous null mutation in Csf2rb2 gene.

In embodiment 93, provided herein is a genetically modified non-human animal embryonic stem cell of embodiment 92, wherein the null mutation in Csf2rb gene is a deletion of the full Csf2rb endogenous coding sequence, and the null mutation in Csf2rb2 gene is a deletion of the full Csf2rb2 endogenous coding sequence.

In embodiment 94, provided herein is a genetically modified non-human animal embryonic stem cell of embodiment 93, wherein the mouse embryonic stem cell comprises a homozygous deletion of nucleic acid sequence between coordinates chr15: 78,282,507-78,351,090 (GRCm38.p6 assembly).

In embodiment 95, provided herein is a genetically modified non-human animal embryonic stem cell of any one of embodiments 86-94, wherein the genetically modified non-human animal embryonic stem cell further comprises a nucleic acid that encodes a human or humanized SIRPA polypeptide, and wherein the nucleic acid is operably linked to a Sirpa promoter.

In embodiment 96, provided herein is a genetically modified non-human animal embryonic stem cell of embodiment 95, wherein the genetically modified non-human animal embryonic stem cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In embodiment 97, provided herein is a genetically modified non-human animal embryonic stem cell of embodiment 96, wherein the Sirpa gene comprises exons 2-4 of a human SIRPA gene.

In embodiment 98, provided herein is a genetically modified non-human animal embryonic stem cell of any one of embodiments 96-97, wherein the non-human animal Sirpa polypeptide is an endogenous non-human animal Sirpa polypeptide, and/or the non-human animal Sirpa gene is an endogenous non-human animal gene.

In embodiment 99, provided herein is a genetically modified non-human animal embryonic stem cell of any one of embodiments 95-98, wherein the genetically modified non-human animal embryonic stem cell further comprises one or more nucleic acids selected from the group consisting of: (1) a nucleic acid that encodes a human TPO protein and is operably linked to a TPO promoter; (2) a nucleic acid that encodes a human M-CSF protein and is operably linked to an M-CSF promoter; and (3) a nucleic acid that encodes a human or humanized CD47 protein and is operably linked to a CD47 promoter.

In embodiment 100, provided herein is a genetically modified non-human animal embryonic stem cell of any one of embodiments 95-99, wherein at least one promoter operably linked to a nucleic acid that encodes a human or humanized protein is an endogenous non-human animal promoter.

In embodiment 101, provided herein is a genetically modified non-human animal embryonic stem cell of embodiment 100, wherein all promoters operably linked to the nucleic acids that encode the human or humanized proteins are endogenous non-human animal promoters.

In embodiment 102, provided herein is a genetically modified non-human animal embryonic stem cell of embodiment 100 or 101, wherein the endogenous non-human animal promoter is at the corresponding non-human animal gene locus.

In embodiment 103, provided herein is a genetically modified non-human animal embryonic stem cell of any one of embodiments 95-102, comprising a null mutation in at least one corresponding non-human animal gene at the corresponding non-human animal gene locus.

In embodiment 104, provided herein is a genetically modified non-human animal embryonic stem cell of any one of embodiments 95-103, wherein the genetically modified non-human animal embryonic stem cell is heterozygous for at least one allele comprising the nucleic acid sequence that encodes the human or humanized protein.

In embodiment 10⁵, provided herein is a genetically modified non-human animal embryonic stem cell of any one of embodiments 95-103, wherein the genetically modified non-human animal embryonic stem cell is homozygous for at least one allele comprising the nucleic acid sequence that encodes the human or humanized protein.

In embodiment 106, provided herein is a genetically modified non-human animal embryonic stem cell of any one of embodiments 86-10⁵, wherein the genetically modified non-human animal embryonic stem cell is a mammalian embryonic stem cell.

In embodiment 107, provided herein is a genetically modified non-human animal embryonic stem cell of embodiment 106, wherein the mammalian embryonic stem cell is a rodent embryonic stem cell, such as a rat embryonic stem cell or a mouse embryonic stem cell.

In embodiment 108, provided herein is a genetically modified non-human animal embryonic stem cell of embodiment 107, wherein the rodent embryonic stem cell is a mouse embryonic stem cell.

In embodiment 109, provided herein is a method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter.

In embodiment 110, provided herein is a method of embodiment 109, wherein the non-human animal embryonic stem cell is further engineered to have a genome that comprises a homozygous null mutation in Rag1 gene.

In embodiment 111, provided herein is a method of embodiment 109 or 110, wherein the non-human animal embryonic stem cell is further engineered to have a genome that comprises a homozygous null mutation in the non-human animal Csf2rb gene.

In embodiment 112, provided herein is a method of any one of embodiments 109-111, wherein the null mutation in the non-human animal Csf2rb gene is a deletion of the full Csf2rb endogenous coding sequence.

In embodiment 113, provided herein is a method of embodiment 110, wherein the non-human animal embryonic stem cell is a mouse embryonic stem cell, and the at least one non-human animal Csf2rb gene comprises mouse Csf2rb gene and/or mouse Csf2rb2 gene.

In embodiment 114, provided herein is a method of any one of embodiments 109-113, wherein the genetically modified non-human animal embryonic stem cell is a mouse embryonic stem cell, and the mouse embryonic stem cell comprises a homozygous null mutation in Csf2rb2 gene.

In embodiment 115, provided herein is a method of embodiment 114, wherein the null mutation in Csf2rb2 gene is a deletion of the full Csf2rb2 endogenous coding sequence.

In embodiment 116, provided herein is a method of embodiment 114 or 115, wherein the mouse embryonic stem cell comprises a homozygous null mutation in Csf2rb gene and a homozygous null mutation in Csf2rb2 gene.

In embodiment 117, provided herein is a method of embodiment 116, wherein the null mutation in Csf2rb gene is a deletion of the full Csf2rb endogenous coding sequence, and the null mutation in Csf2rb2 gene is a deletion of the full Csf2rb2 endogenous coding sequence.

In embodiment 118, provided herein is a method of embodiment 117, wherein the mouse embryonic stem cell comprises a homozygous deletion of nucleic acid sequence between coordinates chr15: 78,282,507-78,351,090 (GRCm38.p6 assembly).

In embodiment 119, provided herein is a method of any one of embodiments 109-118, wherein the genetically modified non-human animal embryonic stem cell is further engineered to comprise in its genome a nucleic acid that encodes a human or humanized SIRPA polypeptide, and wherein the nucleic acid is operably linked to a Sirpa promoter.

In embodiment 120, provided herein is a method of embodiment 119, wherein the genetically modified non-human animal embryonic stem cell comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In embodiment 121, provided herein is a method of embodiment 120, wherein the Sirpa gene comprises exons 2-4 of a human SIRPA gene.

In embodiment 122, provided herein is a method of any one of embodiments 120-121, wherein the non-human animal Sirpa polypeptide is an endogenous non-human animal Sirpa polypeptide, and/or the non-human animal Sirpa gene is an endogenous non-human animal gene.

In embodiment 123, provided herein is a method of any one of embodiments 119-122, wherein the genetically modified non-human animal embryonic stem cell is further engineered to comprise in its genome one or more nucleic acids selected from the group consisting of: (1) a nucleic acid that encodes a human TPO protein and is operably linked to a TPO promoter; (2) a nucleic acid that encodes a human M-CSF protein and is operably linked to an M-CSF promoter; and (3) a nucleic acid that encodes a human or humanized CD47 protein and is operably linked to a CD47 promoter.

In embodiment 124, provided herein is a method of any one of embodiments 119-123, wherein at least one promoter operably linked to a nucleic acid that encodes a human or humanized protein is an endogenous non-human animal promoter.

In embodiment 125, provided herein is a method of embodiment 124, wherein all promoters operably linked to the nucleic acids that encode the human or humanized proteins are endogenous non-human animal promoters.

In embodiment 126, provided herein is a method of embodiment 124 or 125, wherein the endogenous non-human animal promoter is at the corresponding non-human animal gene locus.

In embodiment 127, provided herein is a method of any one of embodiments 119-126, wherein the genetically modified non-human animal embryonic stem cell comprises a null mutation in at least one corresponding non-human animal gene at the corresponding non-human animal gene locus.

In embodiment 128, provided herein is a method of any one of embodiments 119-127, wherein the genetically modified non-human animal embryonic stem cell is heterozygous for at least one allele comprising the nucleic acid sequence that encodes the human or humanized protein.

In embodiment 129, provided herein is a method of any one of embodiments 119-127, wherein the genetically modified non-human animal embryonic stem cell is homozygous for at least one allele comprising the nucleic acid sequence that encodes the human or humanized protein.

In embodiment 130, provided herein is a method of any one of embodiments 109-129, wherein the genetically modified non-human animal embryonic stem cell is a mammalian embryonic stem cell.

In embodiment 131, provided herein is a method of embodiment 130, wherein the mammalian embryonic stem cell is a rodent embryonic stem cell, such as a rat embryonic stem cell or a mouse embryonic stem cell.

In embodiment 132, provided herein is a method of embodiment 131, wherein the rodent embryonic stem cell is a mouse embryonic stem cell.

In embodiment 133, provided herein is a non-human animal embryo comprising the non-human animal embryonic stem cell of any one of embodiments 85-108, or the non-human animal embryonic stem cell made according to the method of any one of embodiments 109-132.

In embodiment 134, provided herein is a method of making a non-human animal comprising in its genome: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising steps of: (a) obtaining a non-human animal embryonic stem cell of any one of embodiments 85-108, or the non-human animal embryonic stem cell made according to the method of any one of embodiments 109-132; and (b) creating a non-human animal using the non-human animal embryonic cell of (a).

In embodiment 135, provided herein is a method of making a non-human animal comprising in its genome: ((i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising modifying the genome of the non-human animal so that it comprises: (i) a homozygous null mutation in Rag2 gene; (ii) a homozygous null mutation in IL2rg gene; and (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter.

In embodiment 136, provided herein is a method of embodiment 135, wherein the genetically modified non-human animal is engineered to comprise a homozygous null mutation in Rag1 gene.

In embodiment 137, provided herein is a method of embodiment 135 or 136, wherein the non-human animal is engineered to comprise a homozygous null mutation in the non-human animal Csf2rb gene.

In embodiment 138, provided herein is a method of any one of embodiments 135-137, wherein the null mutation in the non-human animal Csf2rb gene is a deletion of the full Csf2rb endogenous coding sequence.

In embodiment 139, provided herein is a method of embodiment 135 or 136, wherein the non-human animal is a mouse, and the at least one non-human animal Csf2rb gene comprises mouse Csf2rb gene and/or mouse Csf2rb2 gene.

In embodiment 140, provided herein is a method of any one of embodiments 135-139, wherein the genetically modified non-human animal is a mouse, and the mouse comprises a homozygous null mutation in Csf2rb2 gene.

In embodiment 141, provided herein is a method of embodiment 140, wherein the null mutation in Csf2rb2 gene is a deletion of the full Csf2rb2 endogenous coding sequence.

In embodiment 142, provided herein is a method of embodiment 140 or 141, wherein the mouse comprises a homozygous null mutation in Csf2rb gene and a homozygous null mutation in Csf2rb2 gene.

In embodiment 143, provided herein is a method of embodiment 142, wherein the null mutation in Csf2rb gene is a deletion of the full Csf2rb endogenous coding sequence, and the null mutation in Csf2rb2 gene is a deletion of the full Csf2rb2 endogenous coding sequence.

In embodiment 144, provided herein is a method of embodiment 143, wherein the mouse comprises a homozygous deletion of nucleic acid sequence between coordinates chr15: 78,282,507-78,351,090 (GRCm38.p6 assembly).

In embodiment 145, provided herein is a method of any one of embodiments 135-144, wherein the genetically modified non-human animal expresses a human GM-CSF protein.

In embodiment 146, provided herein is a method of any one of embodiments 135-145, wherein the genetically modified non-human animal expresses a human IL-3 protein.

In embodiment 147, provided herein is a method of any one of embodiments 135-146, wherein the genetically modified non-human animal expresses a human or humanized SIRPA polypeptide encoded by a nucleic acid operably linked to a Sirpa promoter.

In embodiment 148, provided herein is a method of embodiment 147, wherein the genetically modified non-human animal comprises a Sirpa gene that encodes a Sirpa polypeptide comprising an extracellular portion of a human SIRPA polypeptide and an intracellular portion of a non-human animal Sirpa polypeptide, wherein the Sirpa gene is operably linked to a Sirpa promoter.

In embodiment 149, provided herein is a method of embodiment 148, wherein the Sirpa gene comprises exons 2-4 of a human SIRPA gene.

In embodiment 150, provided herein is a method of embodiment 148 or 149, wherein the genetically modified non-human animal expresses a Sirpa polypeptide comprising an extracellular portion of a human SIRPA polypeptide and an intracellular portion of a non-human animal Sirpa polypeptide.

In embodiment 151, provided herein is a method of any one of embodiments 148-150, wherein the non-human animal Sirpa polypeptide is an endogenous non-human animal Sirpa polypeptide, and/or the non-human animal Sirpa gene is an endogenous non-human animal gene.

In embodiment 152, provided herein is a method of embodiment 147, wherein the genetically modified non-human animal expresses a human SIRPA polypeptide encoded by a nucleic acid operably linked to a Sirpa promoter.

In embodiment 153, provided herein is a method of any one of embodiments 147-152, wherein the genetically modified non-human animal further expresses one or more human or humanized proteins selected from the group consisting of: a human TPO protein encoded by a nucleic acid operably linked to a TPO promoter; a human M-CSF protein encoded by a nucleic acid operably linked to an M-CSF promoter; and a human or humanized CD47 protein encoded by a nucleic acid operably linked to a CD47 promoter.

In embodiment 154, provided herein is a method of any one of embodiments 147-153, wherein at least one promoter operably linked to a nucleic acid that encodes a human or humanized protein is an endogenous non-human animal promoter.

In embodiment 155, provided herein is a method of embodiment 154, wherein all promoters operably linked to the nucleic acids that encode the human or humanized proteins are endogenous non-human animal promoters.

In embodiment 156, provided herein is a method of embodiment 154 or 155, wherein the endogenous non-human animal promoter is at the corresponding non-human animal gene locus.

In embodiment 157, provided herein is a method of any one of embodiments 147-156, wherein the genetically modified non-human animal comprises a null mutation in at least one corresponding non-human animal gene at the corresponding non-human animal gene locus.

In embodiment 158, provided herein is a method of any one of embodiments 147-157, wherein the genetically modified non-human animal is heterozygous for at least one allele comprising the nucleic acid sequence that encodes the human or humanized protein.

In embodiment 159, provided herein is a method of any one of embodiments 147-157, wherein the genetically modified non-human animal is homozygous for at least one allele comprising the nucleic acid sequence that encodes the human or humanized protein.

In embodiment 160, provided herein is a method of any one of embodiments 147-159, wherein the genetically modified non-human animal expresses a human M-CSF protein encoded by a nucleic acid operably linked to an M-CSF promoter.

In embodiment 161, provided herein is a method of any one of embodiments 147-160, wherein the genetically modified non-human animal expresses a human or humanized CD47 protein encoded by a nucleic acid operably linked to a CD47 promoter.

In embodiment 162, provided herein is a method of embodiment 161, wherein the genetically modified non-human animal expresses a humanized CD47 protein, and the humanized CD47 protein comprises an extracellular portion of a human CD47 protein and an intracellular portion of an endogenous non-human animal CD47 protein.

In embodiment 163, provided herein is a method of any one of embodiments 147-162, wherein the genetically modified non-human animal expresses a human TPO protein encoded by a nucleic acid operably linked to a TPO promoter.

In embodiment 164, provided herein is a method of any one of embodiments 147-163, wherein the genetically modified non-human animal expresses: (i) a human or humanized SIRPA protein encoded by a nucleic acid operably linked to a Sirpa promoter; (ii) a human M-CSF protein encoded by a nucleic acid operably linked to an M-CSF promoter; (iii) a human or humanized CD47 protein encoded by a nucleic acid operably linked to a CD47 promoter; and (iv) a human TPO protein encoded by a nucleic acid operably linked to a TPO promoter.

In embodiment 165, provided herein is a method of any one of embodiments 135-164, wherein the genetically modified non-human animal is a mammal.

In embodiment 166, provided herein is a method of embodiment 165, wherein the mammal is a rodent, such as a rat or a mouse.

In embodiment 167, provided herein is a method of embodiment 166, wherein the rodent is a mouse.

### EXAMPLES

The following Examples and the accompanying Drawings are provided so as to describe to those of ordinary skill in the art how to make and use methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. The Examples do not include detailed descriptions of conventional methods that would be well known to those of ordinary skill in the art (molecular cloning techniques, etc.). Unless indicated otherwise, parts are parts by weight, molecular weight is average molecular weight, temperature is indicated in Celsius, and pressure is at or near atmospheric.

### Example 1: Generation of Mice with Humanized GM-CSF and IL-3, Humanized SIRPa, Humanized TPO, Rag2−/−, IL-2Rg−/− and a Deletion of the Murine Csf2rb & Csf2rb2 Genes (IStRG/mCSF2Rb KO)

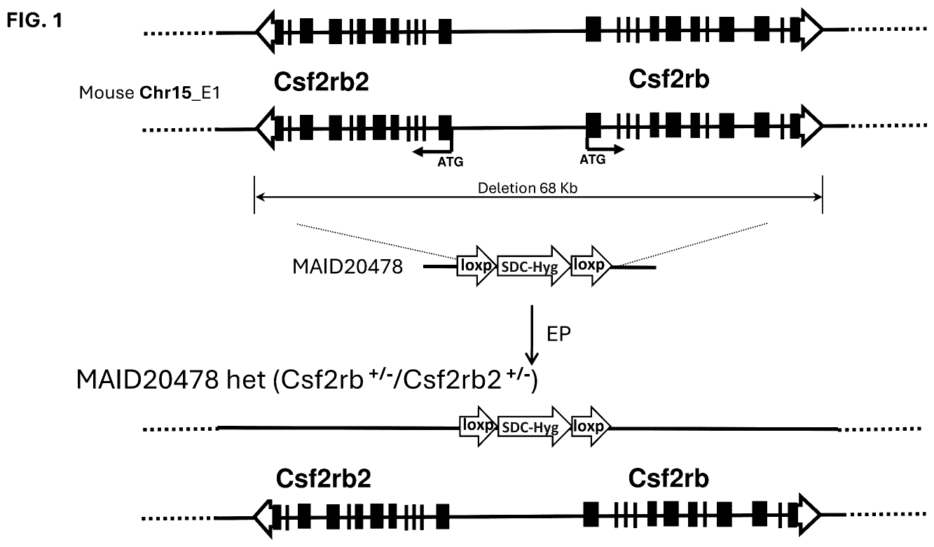

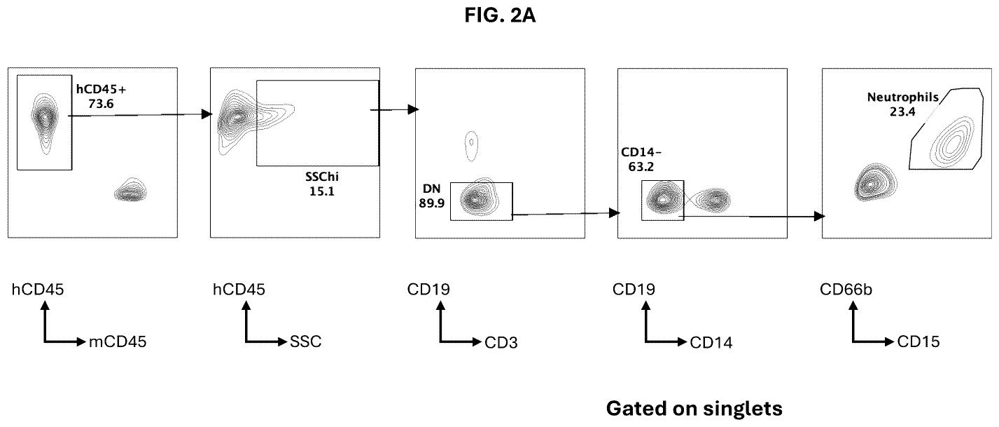

Mouse Csf2rb & Csf2rb2 genes locus was knocked out in the mouse genome using VELOCIGENE® technology (see, e.g., U.S. Pat. No. 6,586,251 and Valenzuela et al. (2003) High-throughput engineering of the mouse genome coupled with high-resolution expression analysis. Nat. Biotech. 21(6): 652-659, both incorporated herein by reference). A 68 kb mouse genomic sequence of Csf2rb & Csf2rb2 genes from the end of Csf2rb gene to the end of Csf2rb2 gene, was deleted on mouse chromosome 15 E1, between coordinates chr15: 78,282,507-78,351,090 (GRCm38.p6 assembly)(FIG. 1).

In detail, mouse homology arms were made by PCR amplification using BAC clone RP23-183P4 as the template, and are indicated in Table 3 below:

TABLE 3

| | | | Coordinates |
|---|---|---|---|
| Homology | | | (GRCm38.p6 |
| Arm | 5′ primer | 3′ primer | assembly) |
| 5′; | CTTCTCTTCTGAGGGG | GCCATGTGTGTGTTTA | chr15: 78,282,360- |
| 147 bp | GTGCAA (SEQ ID NO: 6) | TGTC (SEQ ID NO: 7) | 78,282,506 |
| 3′; | ACAGTTCCAACCCAT | CCTCTGGTTCCTCCTC | chr15: 78,351,091- |
| 143 bp | GGGTG (SEQ ID NO: 8) | CACAAG (SEQ ID NO: 9) | 78,351,233 |

To make the targeting vector (designated MAID20478) from mouse BAC clone RP23-183P4 by the bacterial homologous recombination (BHR) step, a Hygromycin (Hyg) resistance self-deleting cassette (with CRE recombinase controlled by Protamine promoter “SDC-Hyg”) flanked by mutant loxp sites (loxp-hyg-loxp), replaced ˜68 kb mouse sequence containing the mouse Csf2rb & Csf2rb2 genes.

The final targeting vector contained from 5′ to 3′: the chloramphenicol resistance cassette (CM), the 5′ mouse homology arm, the loxp-Hyg-loxp self-deleting cassette, and the 3′ mouse homology arm; the final clone was selected based on CM/Hyg resistance (MAID20478 in FIG. 1).

MAID20478 targeting vector was electroporated into mouse embryonic stem (ES) cells. Targeted homologous recombination resulted in deletion of ˜68 kb of mouse sequence (GRCm38.p6 coordinates chr15: 78,282,507-78,351,090). Successful integration was confirmed by a modification of allele (MOA) assay as described, e.g., in Valenzuela et al, supra. Probes used for the MOA assay for the loss of mouse Csf2rb & Csf2rb2 sequences are depicted in Table 4 below. The hyg cassette was subsequently removed by expression of CRE recombinase (controlled by Protamine promoter) in mice.

TABLE 4

| | | Coordinates |
|---|---|---|
| Probe | | (GRCm38.p6 |
| Name | Probe (5′ to 3′) | assembly) |
| Csf2rb_U | TGAAGACATGTGGCTGCCATCCAG | chr15: |
| | (SEQ ID NO: 10) | 78,282,767- |
| | | 78,282,790 |
| Csf2rb_D | CGTGTCTGCATTGCTATGAGATGGGT | chr15: |
| | (SEQ ID NO: 11) | 78,350,426- |
| | | 78,350,451 |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice with Csf2rb & Csf2rb2 genes deleted were identified by genotyping using the MOA assay described above. Mice heterozygous for KO Csf2rb & Csf2rb2 gene were bred to homozygosity.

Mice comprising deletion of Csf2rb & Csf2rb2 genes can be bred to mice comprising deletion of RAG2 and IL2RG genes, as well as GM-CSF, IL-3 and SIRPa humanizations; additional humanizations described herein may be included. Alternatively, MAID20478 targeting vector can be electroporated into mouse embryonic stem cells comprising deletion of RAG2 and IL2RG genes, as well as GM-CSF, IL-3 and SIRPa humanizations.

Specifically, in one instance, MAID20478 targeting vector was electroporated into mouse embryonic stem cells comprising deletion of RAG2 and IL2RG genes, as well as GM-CSF, IL-3, TPO and SIRPa humanizations. Mice were bred to homozygosity for deletion of Csf2rb & Csf2rb2 genes, deletion of RAG2 and IL2RG genes, as well as to homozygosity for GM-CSF, IL-3 and SIRPa humanizations. Various humanizations are described in, e.g., U.S. Pat. Nos. 10,206,379, 8,541,646, 9,301,509, 9,554,563, and 10,015,953, as well as other patents and patent applications described throughout this disclosure, all incorporated by reference herein in their entireties.

### Example 2: Increased Human Neutrophils in Spleen, Blood, and BM of Human HSC-Engrafted IStRG/mCSF2Rb KO Mice

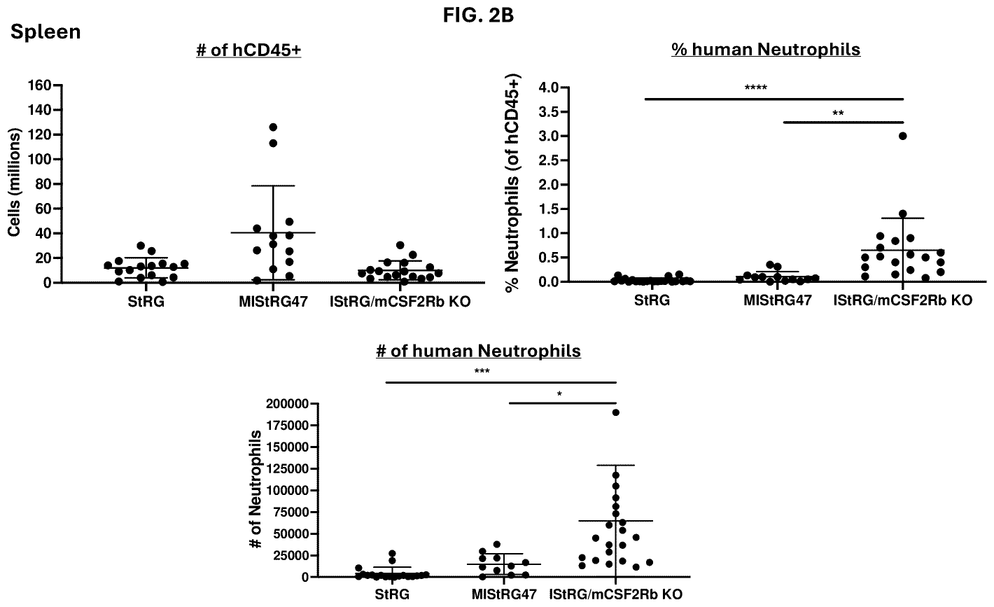

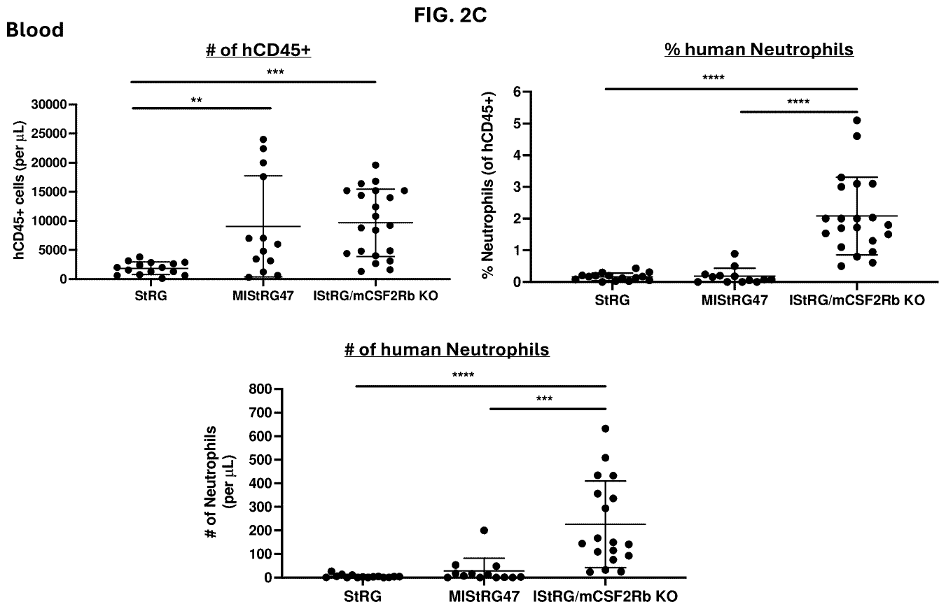

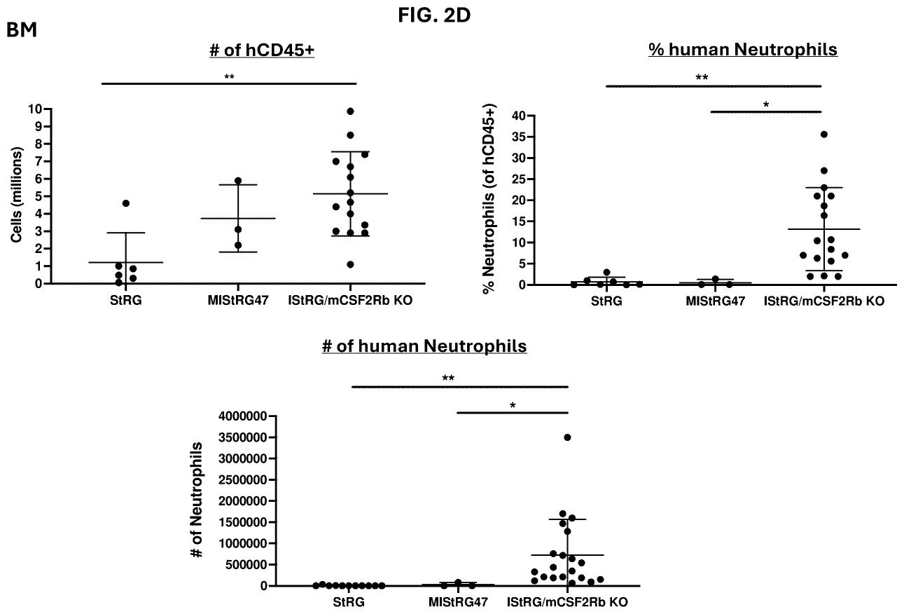

Human neutrophil levels were determined in blood, spleen and BM collected from 3 different Human Immune System (HIS) mouse models: (1) baseline HIS mice with humanized SIRPa, humanized thrombopoietin, Rag2−/− and IL-2Rg−/− (StRG); (2) the advanced HIS model with humanized M-CSF, humanized GM-CSF and IL-3, humanized SIRPa, humanized thrombopoietin, Rag2−/−, IL-2Rg−/− and humanized CD47 (MIStRG47); and (3) the generated HIS model with humanized GM-CSF and IL-3, humanized TPO, humanized SIRPa, Rag2−/−, IL-2Rg−/− and a deletion of the murine Csf2rb & Csf2rb2, mouse receptor for GM-CSF/IL-3 (IStRG/mCSF2Rb KO). All mouse strains were engrafted by injection of 100,000 hHSCs injected intra-hepatically into 1-5 day-old pups. 10-12 weeks later, mice were bled by cardiac puncture and spleens/BM were harvested. Single cell suspension was prepared by mechanical disruption of spleen/BM, passage through a 70 mM mesh filter, followed by lysis of red blood cells (RBCs) in ACK lysing buffer (Gibco). Blood single cell suspension was prepared by RBC lysis in ACK lysing buffer (Gibco). All cells were resuspended in FACS buffer with mouse/human Fc block (BD Biosciences) and counted. Cells were stained for FACS analysis with the following monoclonal antibodies at a 1:50 dilution in PBS+1 mM EDTA and 2% fetal bovine serum (FACS buffer): anti-mouse CD45-APC-Cy7 (clone 30-F11; BD Biosciences), anti-human CD45-PE-Cy5.5 (clone HI30; ThermoFisher), anti-human CD19-PE-Cy7 (clone HIB19; BD Biosciences), anti-human CD3-Pacific Blue (clone S4.1; ThermoFisher), anti-human CD14 PE-Cy7 (clone M5E2; BD Biosciences), anti-human CD66b FITC (clone G10F5; Biolegend), and anti-human CD15 PE-Dazzle (clone W6D3; Biolegend). Gating strategy for human neutrophils is shown in FIG. 2A. Briefly, hCD45+/mCD45− cells were gated for high side scatter, and of those, CD3−/CD19− double negative (DN) cells were gated (FIG. 2A). Of those, CD14− cells (non-myeloid) were gated on, and CD15+/CD66b+ cells were distinguished as human neutrophils (FIG. 2A). Engrafted IStRG/mCSF2Rb KO mice had increased human neutrophils in both spleen (FIG. 2B), blood (FIG. 2C) and BM (FIG. 2D). In blood, engrafted IStRG/mCSF2Rb KO mice averaged >250 neutrophils per p L which was >40× more than the other HIS models despite having comparable engraftment levels (FIG. 2C).

### Example 3: Human Neutrophils from Human HSC-Engrafted IStRG/mCSF2Rb KO Mice Express CD89 (FcαR), MPO and CD177

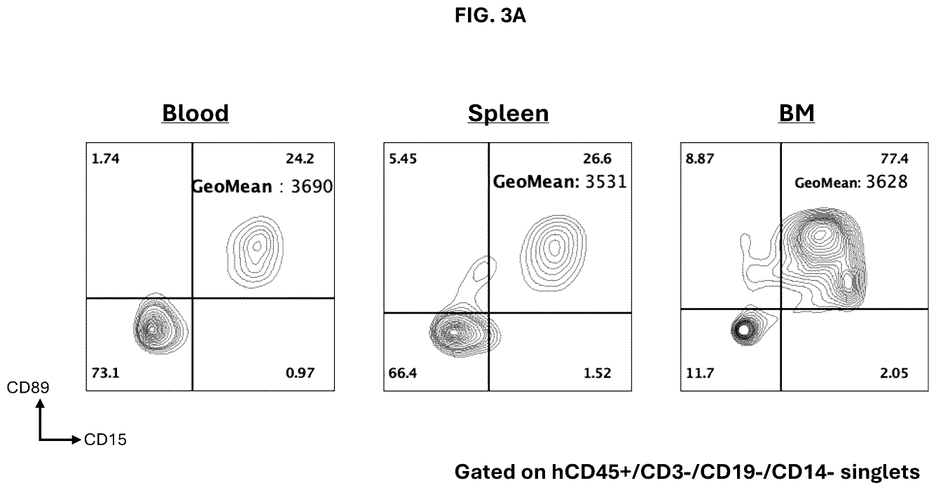

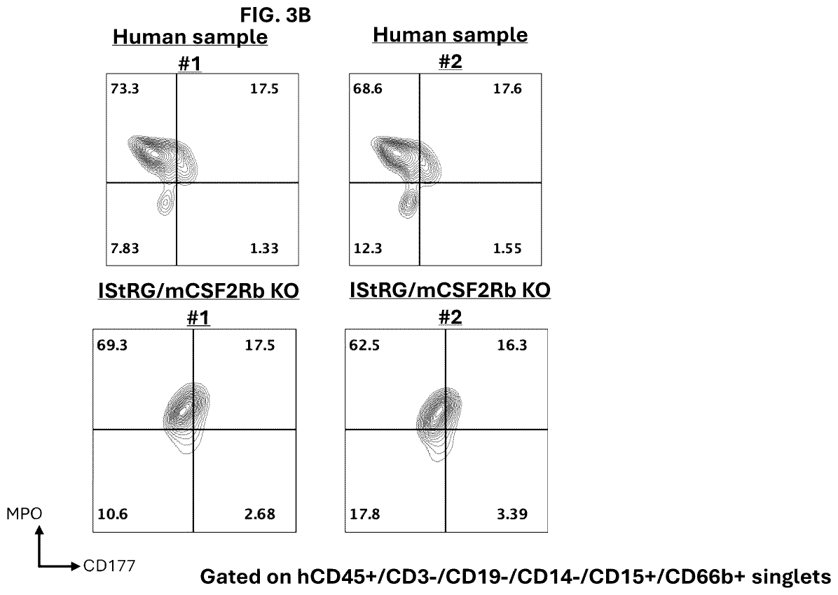

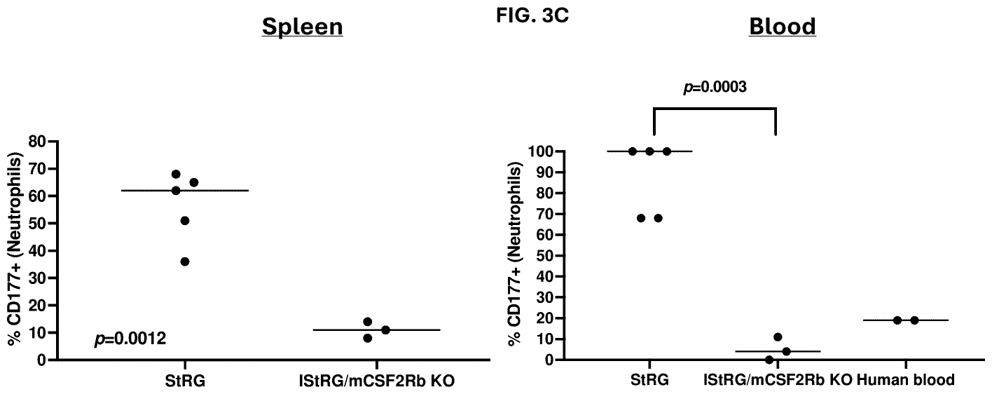

In order to validate that human neutrophils from engrafted IStRG/mCSF2Rb KO mice are comparable to normal human neutrophils, several key molecules on human neutrophils were analyzed. Normal human neutrophils express high levels of CD89 (FcαR) which binds to IgA antibody and 1) potentiates phagocytosis of IgA-opsonized pathogens, and 2) triggers release of reactive oxygen intermediates by neutrophils, e.g., produces a superoxide burst. Neutrophils from human HSC-engrafted IStRG/mCSF2Rb KO mice as described above were further analyzed with anti-human CD89 BV650 (clone A59; BD Biosciences). All CD15+ neutrophils from the blood, spleen, and BM of engrafted IStRG/mCSF2Rb KO were positive for CD89 (FIG. 3A).

CD177 is a GPI-linked membrane protein that mediates neutrophil transmigration through endothelial cells and is also reported to interact with various secreted proteases such as Proteinase 3 (PR3). CD177 is generally up-regulated on activated neutrophils. Myeloperoxidase (MPO) is an intracellular enzyme that neutrophils specifically contain to trigger superoxide production. Using anti-human CD177 BUV395 (clone MEM-166; BD Biosciences) and anti-human MPO PE (clone 5B8; BD Biosciences), it was demonstrated herein that human neutrophils from human HSC-engrafted IStRG/mCSF2Rb KO mice expressed comparable CD177 and MPO to neutrophils from normal human blood (FIG. 3B). In contrast, human HSC-engrafted StRG mice had abnormally higher percentage of CD177+ neutrophils in their blood, albeit lower overall cells, than blood neutrophils from donor human HSC-matched IStRG/mCSF2Rb KO mice or neutrophils from normal human blood (FIG. 3C). Notably, neutrophils in spleen of StRG mice also expressed higher CD177 than splenic neutrophils in same donor human HSC-engrafted IStRG/mCSF2Rb KO mice (FIG. 3C).

### Example 4: Comparable Human Neutrophil Development in Human HSC-Engrafted IStRG/mCSF2Rb KO BM as in Normal Human BM

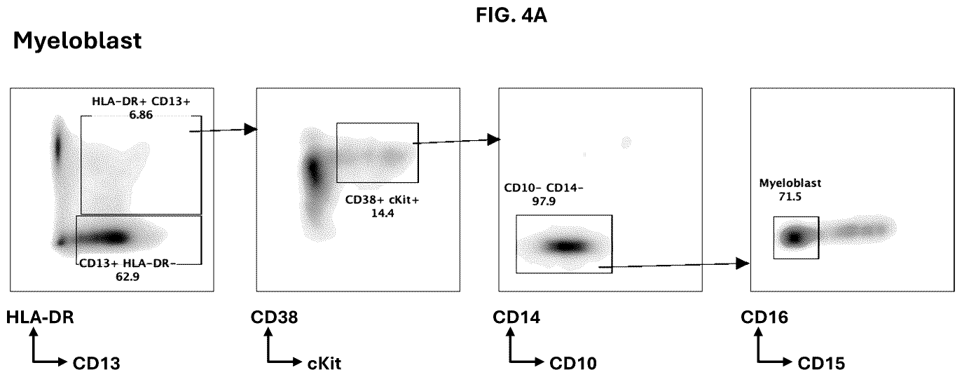

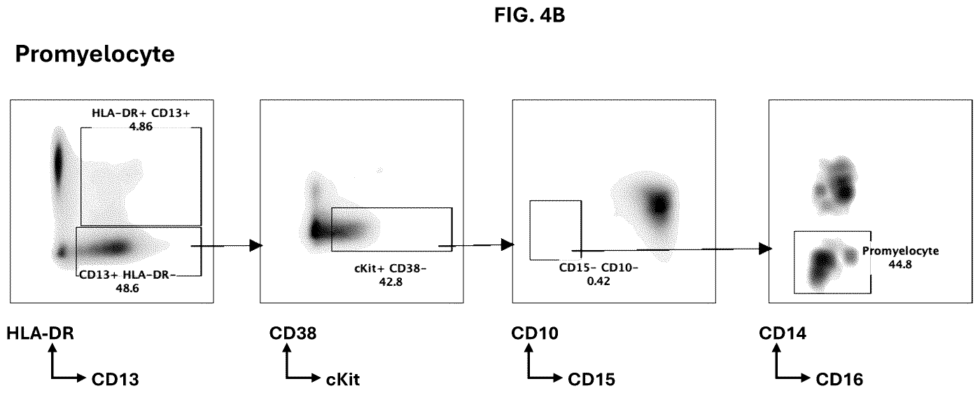

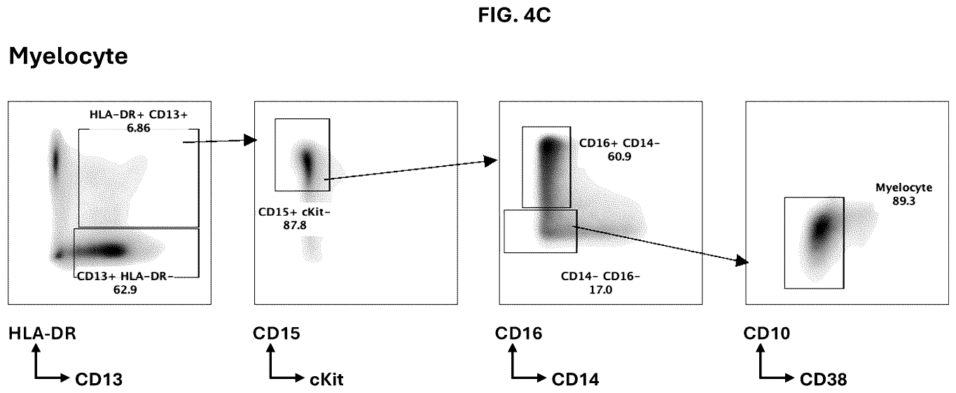

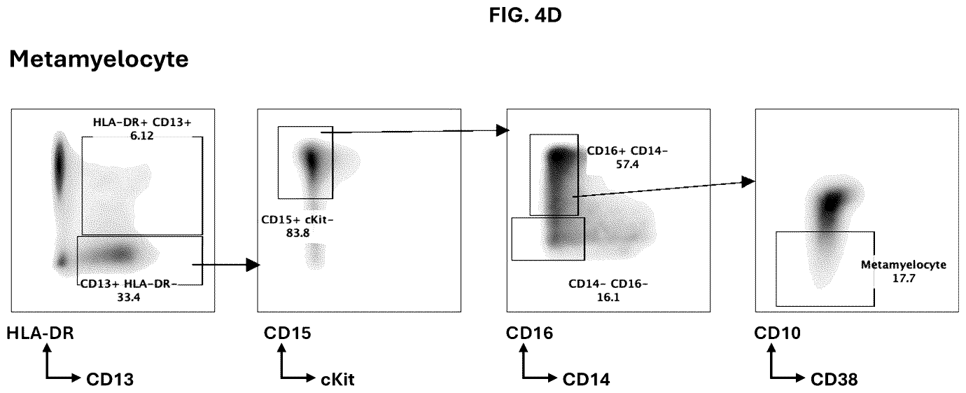

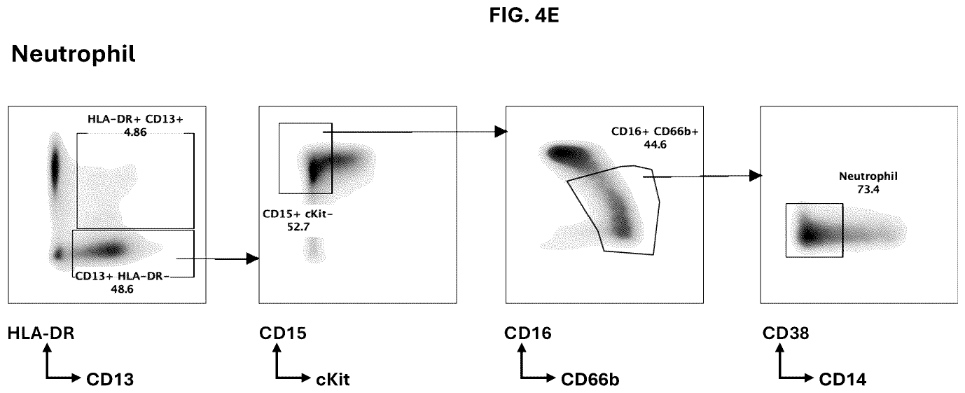

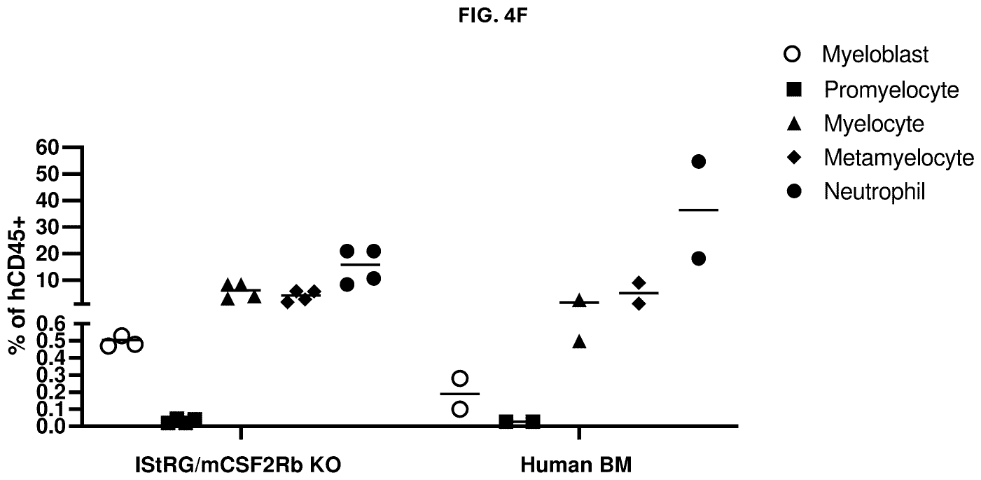

In order to assess granulocyte maturation in the BM of human HSC-engrafted IStRG/mCSF2Rb KO mice, BM was harvested and FACS analysis of the single cell suspension was performed with the following antibodies: anti-mouse CD45-APC-Cy7 (clone 30-F11; BD Biosciences), anti-human CD45-PE-Cy5.5 (clone HI30; ThermoFisher), anti-human CD14 PE-Cy7 (clone M5E2; BD Biosciences), anti-human CD66b FITC (clone G10F5; Biolegend), anti-human CD15 PE-Dazzle (clone W6D3; Biolegend), anti-human HLA-DR PE (clone Tu36; Biolegend), anti-human CD16 Pacific Blue (clone 3G8; Biolegend), anti-human CD13 BUV395 (clone WM15; BD Biosciences), anti-human cKit APC (clone 104D2; Biolegend), anti-human CD10 BV605 (clone HI10A; Biolegend), and anti-human CD38 FITC (clone HB7; BD Biosciences). Granulocyte precursors were distinguished as follows: Myeloblast (hCD45+/mCD45− CD13+ HLA-DR+ ckit+ CD38+ CD10− CD14− CD15− CD16−) (FIG. 4A), Promyelocyte (hCD45+/mCD45− CD13+ HLA-DR− ckit+ CD38− CD10− CD14− CD15− CD16−) (FIG. 4B), Myelocyte (hCD45+/mCD45− CD13+ HLA-DR+ ckit− CD38− CD10− CD14− CD15+ CD16−) (FIG. 4C), Metamyelocyte (hCD45+/mCD45− CD13+ HLA-DR− ckit− CD38− CD10− CD14− CD15+ CD16+) (FIG. 4D), and mature neutrophil as being hCD45+/mCD45− CD13+ HLA-DR− ckit− CD38− CD10+ CD14− CD15+ CD16+ CD66b+ (FIG. 4E). The percentages of different granulocytic precursors (as percentage of hCD45+ leukocytes) in human HSC-engrafted IStRG/mCSF2Rb KO BM were comparable to those of normal human BM samples (FIG. 4F), albeit with slightly higher percentage of myeloblast precursors in IStRG/mCSF2Rb KO BM (FIG. 4F). Expectedly, normal human BM also had a higher frequency of mature neutrophils versus the BM of human HSC-engrafted IStRG/mCSF2Rb KO mice (FIG. 4F).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov. A person of ordinary skill in the art would understand that the sequence databases disclosed above or those known in the art are periodically updated to publish corrected sequences. Incorporated by reference in their entirety are such corrected sequences.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1.-91. (canceled)

92. A genetically modified non-human animal cell, comprising:
(i) a homozygous null mutation in Rag2 gene;
(ii) a homozygous null mutation in IL2rg gene;
(iii) a homozygous null mutation in at least one non-human animal colony stimulating factor 2 receptor subunit beta (Csf2rb) gene;
(iv) a nucleic acid sequence that encodes a human GM-CSF and is operably linked to a GM-CSF promoter; and
(v) a nucleic acid sequence that encodes a human IL-3 and is operably linked to an IL-3 promoter.

93. The genetically modified non-human animal cell of claim 92 comprising a homozygous null mutation in Rag1 gene.

94. The genetically modified non-human animal cell of claim 92, wherein the genetically modified non-human animal cell is a mouse cell, and the mouse cell comprises a homozygous null mutation in Csf2rb gene and a homozygous null mutation in Csf2rb2 gene.

95. The genetically modified non-human animal cell of claim 94, wherein the null mutation in Csf2rb gene is a deletion of the full Csf2rb endogenous coding sequence, and the null mutation in Csf2rb2 gene is a deletion of the full Csf2rb2 endogenous coding sequence.

96. The genetically modified non-human animal cell of claim 95, wherein the mouse cell comprises a homozygous deletion of nucleic acid sequence between coordinates chr15: 78,282,507-78,351,090 (GRCm38.p6 assembly).

97. The genetically modified non-human animal cell of claim 92, wherein the genetically modified non-human animal cell expresses a human GM-CSF protein and/or a human IL-3 protein.

98. The genetically modified non-human animal cell of claim 92, wherein the genetically modified non-human animal cell further comprises a nucleic acid that encodes a human or humanized SIRPA polypeptide, and wherein the nucleic acid is operably linked to a Sirpa promoter.

99. The genetically modified non-human animal cell of claim 98, wherein the genetically modified non-human animal cell further comprises one or more nucleic acids selected from the group consisting of:
(1) a nucleic acid that encodes a human TPO protein and is operably linked to a TPO promoter;
(2) a nucleic acid that encodes a human M-CSF protein and is operably linked to an M-CSF promoter; and
(3) a nucleic acid that encodes a human or humanized CD47 protein and is operably linked to a CD47 promoter.

100. The genetically modified non-human animal cell of claim 92, wherein the genetically modified non-human animal cell is a rodent cell, such as a rat cell or a mouse cell.

101. The genetically modified non-human animal cell of claim 92, wherein the genetically modified non-human animal cell is a non-human animal embryonic stem (ES) cell.

102. A genetically modified non-human animal, comprising:
(i) a homozygous null mutation in Rag2 gene;
(ii) a homozygous null mutation in IL2rg gene;
(iii) a homozygous null mutation in at least one non-human animal colony stimulating factor 2 receptor subunit beta (Csf2rb) gene;
(iv) a nucleic acid sequence that encodes a human GM-CSF and is operably linked to a GM-CSF promoter; and
(v) a nucleic acid sequence that encodes a human IL-3 and is operably linked to an IL-3 promoter.

103. The genetically modified non-human animal of claim 102, wherein the genetically modified non-human animal comprises human neutrophils.

104. The genetically modified non-human animal of claim 103, wherein anti-neutrophil cytoplasmic autoimmune (ANCA) vasculitis is established in the genetically modified non-human animal.

105. The genetically modified non-human animal of claim 103, wherein Granulomatosis with polyangiitis (GPA) is established in the genetically modified non-human animal.

106. The genetically modified non-human animal of claim 103, wherein a tumor is established in the genetically modified non-human animal.

107. The genetically modified non-human animal of claim 103, wherein the genetically modified non-human animal is infected by a bacterial or fungal pathogen.

108. The genetically modified non-human animal of claim 103, wherein human neutrophil NETosis is established in the genetically modified non-human animal.

109. The genetically modified non-human animal of claim 108, wherein the genetically modified non-human animal has an autoimmune disease that involves human neutrophil NETosis.

110. A method for identifying an agent that treats anti-neutrophil cytoplasmic autoimmune (ANCA) vasculitis, the method comprising:
(a) administering the agent to a genetically modified non-human animal of claim 104; and
(b) determining whether the agent treats ANCA vasculitis in the non-human animal.

111. A method for identifying an agent that treats Granulomatosis with polyangiitis (GPA), the method comprising:
(a) administering the agent to a genetically modified non-human animal of claim 105; and
(b) determining whether the agent treats GPA in the non-human animal.

112. A method for identifying an agent that mitigates tumor progression, the method comprising:
(a) administering the agent to a genetically modified non-human animal of claim 106; and
(b) determining whether the agent mitigates tumor progression in the non-human animal.

113. A method for identifying an agent that inhibits an infection by a bacterial or fungal pathogen, the method comprising:
(a) administering the agent to a genetically modified non-human animal of claim 107, and
(b) determining whether the agent reduces the amount of the pathogen and/or inhibits the activity of the pathogen in the pathogen-infected non-human animal.

114. A method for identifying an agent that mitigates neutrophil NETosis, the method comprising:
(a) administering the agent to a genetically modified non-human animal of claim 108; and
(b) determining whether the agent mitigates neutrophil NETosis in the non-human animal.

115. A method for identifying an agent that treats an autoimmune disease that involves neutrophil NETosis, the method comprising:
(a) administering the agent to a genetically modified non-human animal of claim 109; and
(b) determining whether the agent treats the autoimmune disease in the non-human animal.

116. A method for assessing therapeutic efficacy of a drug candidate to modulate a function of a human neutrophil, the method comprising:
(a) administering the drug candidate to a genetically modified non-human animal of claim 103; and
(b) determining whether the drug candidate modulates the function of the human neutrophil in the genetically modified non-human animal.

117. A method of making a non-human animal embryonic stem cell, comprising genetically engineering the non-human animal embryonic stem cell so that the non-human animal embryonic stem cell has a genome that comprises: (i) a homozygous null mutation in Rag2 gene; (ii) a homozygous null mutation in IL2rg gene; (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter.

118. A non-human animal embryo comprising the non-human animal embryonic stem cell of claim 101.

119. A method of making a non-human animal comprising in its genome: (i) a homozygous null mutation in Rag2 gene; (ii) a homozygous null mutation in IL2rg gene; (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising steps of:
(a) obtaining a non-human animal embryonic stem cell of claim 101; and
(b) creating a non-human animal using the non-human animal embryonic cell of (a).

120. A method of making a non-human animal comprising in its genome: (i) a homozygous null mutation in Rag2 gene; (ii) a homozygous null mutation in IL2rg gene; (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter, the method comprising modifying the genome of the non-human animal so that it comprises: (i) a homozygous null mutation in Rag2 gene; (ii) a homozygous null mutation in IL2rg gene; (iii) a homozygous null mutation in at least one non-human animal Csf2rb gene; (iv) a nucleic acid sequence that encodes a human GM-CSF and is operably linked to a GM-CSF promoter; and (v) a nucleic acid sequence that encodes a human IL-3 and is operably linked to a IL-3 promoter.
